(19) United States
(12) Patent Application Publication
(10) Pub. No.: US 2015/0307846 A1
(43) Pub. Date: Oct. 29, 2015

(54) GROWTH FACTOR COCKTAIL TO ENHANCE OSTEOGENIC DIFFERENTIATION OF MESENCHYMAL CELLS

(71) Applicants: University of Pennsylvania, Philadelphia, PA (US); Katholieke Universiteit Leuven, Leuven (BE)

(72) Inventors: Christopher Chen, Princeton, NJ (US); Frank Luyten, Kraainem (BE); Jeroen Eyckmans, Philadelphia, PA (US); Jan Schrooten, Leuven (BE); Scott Roberts, Leuven (BE)

(73) Assignees: Katholieke Universiteit Leuven, Leuven (BE); University of Pennsylvania, Philadelphia, PA (US)

(21) Appl. No.: 14/408,416

(22) PCT Filed: Jun. 19, 2013
(86) PCT No.: PCT/EP2013/062725

§ 371 Date: Dec. 16, 2014

Related U.S. Application Data
(60) Provisional application No. 61/661,448, filed on Jun. 19, 2012.

(30) Foreign Application Priority Data
Jul. 31, 2012 (GB) 1213571.1

(51) Int. Cl. C12N 5/077; A61K 45/06; A61K 35/12

(52) U.S. Cl. CPC C12N 5/0654; A61K 35/12; A61K 45/06; C12N 2501/11; C12N 2501/2306; C12N 2501/15; C12N 2500/14; C12N 2500/38; C12N 2501/25; C12N 2506/13

(57) ABSTRACT

The present invention relates to methods and compositions for osteogenic differentiation of human periosteum derived cells, in particular using a growth medium containing a specific combination of growth factors and formulations thereof. The invention also relates to the differentiated cells and cell populations, as well as further products comprising such cells and uses thereof in bone therapy.

### FIELD OF THE INVENTION

The present invention relates to methods and compositions for osteogenic differentiation of human periosteum derived cells, in particular using a growth medium containing a specific combination of growth factors and formulations thereof. The invention also relates to the differentiated cells and cell populations, as well as further products comprising such cells and uses thereof in bone therapy.

In particular, the present invention relates to methods for culturing cells, more particularly mesenchymal cells such as human periosteum derived cells, to enhance bone formation. The present invention more specifically relates to inducing osteogenic differentiation of cells in a growth medium formulation containing a specific combination of growth factors. The present invention has applications in the areas of cell culture, drug discovery (development of bone formation assays), orthopedic surgery, tissue engineering, and bone fracture healing.

### BACKGROUND OF THE INVENTION

Five percent of bone fractures do not heal naturally and require surgical intervention to stabilize the fracture. The gold standard to promote healing of non union fractures is transplantation of autologous bone graft harvested from the iliac crest into the defect. Several complications, such as donor site morbidity have driven the field to explore alternative approaches. Indeed, major efforts to heal non union defects with cell therapeutics or bone tissue engineering techniques are currently undertaken. The healing of fractured bone is strongly dependent on osteoinduction, a process that commences with the recruitment and proliferation of immature multipotent cells followed by differentiation into chondroblasts and/or osteoblasts. Once committed to the osteogenic lineage, osteoblasts secrete bone matrix and in concert with mineralizing chondrocytes repair the fractured site. Because osteoinduction can occur in heterotopic and ectopic sites, the process does not necessarily require the proximity of native bone tissue to happen. Hence, the standard assay to test osteoinductive properties of agents has been injection or implantation of materials carrying the agents in a soft tissue pouch under the kidney cap, in skeletal muscle or subcutaneously in immune compromised mice or rats. Utilizing an ectopic assay, Urist discovered that three weeks after implantation demineralized bone matrix is revascularized and de novo bone formation occurs through the endochondral route. Subsequent research identified a soluble glycoprotein named Bone Morphogenetic Protein (BMP) potent to induce endochondral bone formation in soft tissue in vivo. Since then, more than 30 members of the BMP family have been characterized and several of them are potent bone inducers in vivo. To date, no other proteins have been found to display such osteoinductive capacity similar to BMPs. Therefore it is not surprising that research scrutinizing the molecular signaling pathways that drive osteoinduction has been mainly BMP centered. Both cell therapeutics and bone tissue engineering techniques aim at increasing proliferation, differentiation, and matrix production of osteogenic committed mesenchymal stem cells (MSCs) upon delivery into the defect, either by injection or loaded on a carrier structure. To differentiate human MSCs towards the osteogenic lineage, cells are treated with growth medium supplemented with dexamethasone, beta glycerophosphate and ascorbic acid (1, 2). This osteogenic medium (OM) has been optimized for bone marrow derived stem cells (BMC) (3) but is inconsistent to induce in vitro osteogenesis in human Periosteum Derived Cells (hPDCs) (4, 5). Moreover, stimulation of hBMCs and hPDCs with other potent osteoinductive growth factors, such as Bone Morphogenetic Proteins (BMPs), also result in limited osteogenic differentiation as compared to their murine homologues. As such, there is an unmet need to have a medium that robustly induces proliferation and osteogenic differentiation in human MSCs.

During isolation of BMPs, it became apparent that these proteins have a high affinity for hydroxyapatite, a crystalline conformation of calcium phosphate (CaP) which is abundantly present in mineralized bone tissue. Intriguingly, porous CaP structures display bone spicules upon intramuscular implantation in large animals such as goat, sheep and baboons suggesting that CaP also can induce ectopic bone formation. This spontaneous bone formation, however, has been less frequently observed in small animals. In contrast, robust ectopic bone formation is obtained in mice when CaP carriers are loaded with mesenchymal stem cell (MSCs) populations derived from cartilage, synovium, periosteum, bone marrow and adipose tissue. Despite the growing body of evidence that CaP can induce osteogenesis in MSCs, the molecular mechanism remains elusive.

### SUMMARY OF THE INVENTION

The invention is based on methods developed by the inventors to produce cells with an osteogenic phenotype in vitro. Cell culture conditions were developed based on gene expression analyzed by genome wide analysis of hPDCs engrafted on decalcified and non-decalcified Collagraft™ carriers before and after subcutaneous implantation in nude mice. The inventors developed specific cell culture conditions to successfully proliferate and differentiate cells that express osteogenic phenotypes. Numbered statements of the invention are as follows.

1. A method for inducing cells to proliferate and differentiate into cells with a osteogenic phenotype, comprising culturing cells in a medium comprising about 2 ng/ml to about 200 ng/ml EGF, about 1 ng/ml to about 100 ng/ml IL6, and about 1 ng/ml to about 100 ng/ml TGFβ1.

2. The method of statement 1, wherein the medium comprises about 20 ng/ml EGF, about 10 ng/ml IL6, and about 10 ng/ml TGFβ1.

3. The method of statements 1 or 2, wherein the medium contains a calcium ion concentration ranging from about 0.3 mM to about 12 mM.

4. The method of statement 3, wherein the medium contains a calcium ion concentration of about 3 mM.

5. The method of any one of statements 1 to 4, wherein the medium contains a serum concentration ranging from 0% to about 20%.

6. The method of statement 5, wherein the medium contains a serum concentration of about 10%.

7. The method of any one of statement 1 to 6, wherein the medium contains about 10⁻⁴ M to about 10⁻⁷ M ascorbic acid.

8. The method of statement 7, wherein the medium contains a concentration of about 50 μM ascorbic acid.

9. The method of any one of statement 1 to 8, wherein the medium contains a phosphate ion concentration ranging from about 0.2 mM to about 8 mM.

10. The method of statement 9, wherein the medium contains a phosphate ion concentration of about 2 mM.

11. The method of any one of statements 1 to 10, wherein the cells are cultured for at least four days.

12. The method of any one of statements 1 to 10, wherein the cells are cultured for 11 days.

13. The method of any of statements 1 to 12, wherein the cells are cultured in a medium which additionally comprises TNFα in a first period, wherein said first period is maximum 4 days.

14. The method of statement 13, wherein the first period is 1, 2, or 3 days.

15. The method of any one of statements 1 to 14, wherein the cells that are contacted with EGF, IL6 and TGFβ1 are stem cells.

16. The method of statement 15, wherein the stem cells are mesenchymal cells.

17. The method of statement 16, wherein the stem cells are periosteum derived cells.

18. The method of any one of statements 1 to 17, wherein the cells are mammalian cells.

19. The method of statement 18, wherein the cells are human cells.

Any eukaryotic cell can be used in the initial step (a) of culturing cells as long as it has a phenotype of a cell that is a primitive mesenchymal phenotype. Such a cell could express membrane markers such as CD73, CD90 or CD105, transcription factors such as PRX1/2 or cytoskeletal elements such as nestin and aSMA (alpha smooth muscle actin) and display multipotent differentiation capacity under standard in vitro conditions as known to a person skilled in the art. For stem cells, for example embryonic stem cells or reprogrammed somatic cells (IPSC) or partially reprogrammed somatic cells, it is required that such stem cells are first differentiated to such a primitive mesenchymal phenotype. At that moment, these differentiated cells can be used according to the methods of the present invention. The whole method, including such pre-differentiation of such stem cells together with the proliferation and differentiation methods as described in detail in this invention, are contemplated in the present invention. In one embodiment, such cells to be used in step (a) express at least 1, 2, 3, 4, 5, 6, 7, 8 or 9 markers selected from the list containing: CD90, CD44, CD105, CD146, CD73, CD166, nestin, αSMA and PRX1 and are negative for one or more of CD34, CD45 and CD14. In one embodiment such cells to be used in step (a) are cells that are derived from neural crest and meso-endodermal lineage during development. Such cells include but are not limited to hematopoietic (stem) cells and other stem cells derived from neural crest.

20. The method of any one of statements 1 to 19, wherein said method is an in vitro method.

21. Cells produced according to any one of the methods recited in the preceding statements.

22. A composition, comprising cells that express a primitive mesenchymal phenotype in a culture medium comprising about 2 ng/ml to about 200 ng/ml EGF, about 1 ng/ml to about 100 ng/ml IL6 and about 1 ng/ml to about 100 ng/ml TGβ1.

23. The composition of statement 22, wherein the medium is comprised of about 20 ng/ml EGF, about 10 ng/ml IL6 and about 10 ng/ml TGFβ1.

24. The composition of statements 22 or 23, wherein the medium further comprises serum in a concentration from 0% to about 20%.

25. The composition of statement 24, wherein the serum concentration is about 10%.

26. The composition of any one of statements 22 to 25, wherein the medium further comprises about 10⁻⁴ M to about 10⁻⁷ M ascorbic acid.

27. The composition of statement 26, wherein the concentration of ascorbic acid is about 50 μM.

28. The composition of any one of statements 22 to 27, wherein the cells are mammalian cells.

29. The composition of any one of statements 22 to 28, wherein the cells are human cells.

30. A pharmaceutical composition comprising the cells produced according to any one of the methods recited in the preceding statements.

31. A method of treatment comprising administering a therapeutically effective amount of the cells produced according to any one of the methods recited in the preceding statements to a subject with a bone disorder.

32. The composition according to any one of statements 22 to 29 for use in medicine

33. The composition according to any one of statements 22 to 29 for use in the treatment of a subject having a bone disorder

34. The method of claim 31 or the composition for use as defined in any one of claims 32-33, wherein said bone disorder is a bone fracture or a non healing bone defect.

35. The method of claim 31 or the composition for use as defined in any one of claims 32-33, wherein the subject is a human patient.

36. The method of any of claims 31 or the composition for use as defined in any one of claims 32-33, further comprising administering non-cellular material to said subject.

37. The method of claim 36 or the composition for use as defined in claim 36, wherein the cells and the non-cellular material are combined in vitro to form an implantable graft.

The invention is also related to pharmaceutical compositions containing the cells of the invention. Such compositions are suitable for administration to subjects in need of such cells. The cells would be administered in therapeutically effective amounts.

The invention is also directed to methods of using the cells produced by the methods of the present invention for the treatment of bone disorders, in particular bone fractures, more particularly non union fractures (bone fractures that do not heal naturally).

The invention is also directed to methods of using the cells for studies of 2 dimensional (2D) and 3 dimensional (3D) in vitro and in vivo bone formation, to identify extra conditions, including identifying additional and replacement growth factor medium components in order to optimize the methods, protocols and assays described in the present invention.

In one embodiment, the cells with an osteogenic phenotype produced according to the method of the present invention can be used as cell therapy or for tissue regeneration in disorders such as but not limited to bone defects and osteoporosis, Paget's disease, bone fracture, osteomyelitis, osteonecrosis, achondroplasia, or osteogenesis imperfecta.

### BRIEF DESCRIPTION OF THE FIGURES

FIG. 1: A) Average gene expression of Osterix (OSX), Bone Sialo Protein (BSP) and Osteocalcin (OC) as measured with Taqman PCR (n=3 donors). Increased expression of BSP and OC at 18 days set 18 days as the last time point for the microarray study. B) Signature of known osteoblast markers at 18 days in CPDM (decalcified Collagraft™) and CPRM (Collagraft™) indicates that osteogenic differentiation occurred within three weeks after implantation. The markers shown in italic are used in subsequent experiments to confirm in vitro osteogenic differentiation in hPDCs.

FIG. 2: A) Self Organizing Maps showing gene topologies of GOI at 20h after seeding and 2, 8 and 18 days after implantation in CPDM and CPRM. Gene expression is normalized to expression in hPDCs seeded on tissue culture plastic for 20h. B) Gene ontology analysis of the 001 indicating the most prominent biological processes that occur in CPRM but not in CPDM at indicated time points.

FIG. 3: A) Average gene expression of co-expressed genes organized in superclusters plotted over time. Solid line: CPRM, Dashed line: CPDM. B) Hub genes from each supercluster are mapped into a single hub gene network. The hub genes are connected with direct (solid lines) and indirect (dashed lines) interactions. The encircled hub genes are probed with western blot to validate differential activation between CPRM and CPDM (FIG. 7). C) Quantification of western blots of p-pERK (MAPK signaling), p-p53, p-Smad 1/5/8 (BMP signaling), p-Smad 2 (TGFβ signaling), p-CREB (cAMP and

EGF signaling), p-NFκB (TNFα/NFxB signaling), and p-β catenin (β-catenin/Wnt signaling). Densitometry values are normalized to GAPDH. For all time points, fold increase is compared to the expression in CPDM at two days (n=3 donors, error bars: standard error of the mean).

FIG. 4: A) Growth factor medium promotes proliferation up to 7 population doublings in 10 days, whereas hPDCs treated with OM reach 5.5 population doublings after 21 days. Note that y-axis representing the number of population doublings is a logarithmic scale. Hence, there are three times more cells when treated with GF mix as compared to OM (GM=Growth medium, OM=Osteogenic medium, GF=Growth Factor medium). B) Relative gene expression of bone markers of hPDCs after treatment with OM and GF for 11 days. Gene expression is normalized to the gene expression in the GM condition (COL1=Collagen type I, ALP=Alkaline Phosphatase, OCN=osteocalcin, DLX5=Distal-less homeobox 5, RUNX2=Runt related transcription factor 2, CADH11=osteoblast specific cadherin 11, SPP1=osteonectin, RANKL=Rank ligand, OSX=Osterix, BMP2=Bone Morphogenetic Protein 2, BSP=Bone Sialo Protein). C) Translation of in vitro matured osteoblasts to a subcutaneous in vivo environment. hPDCs were seeded on 21mm3 CPRMs at a density of 1×10⁶ cells before 10 days of treatment in GM containing the GF cocktail [ascorbic acid (57 μM), IL6 (10 ng/ml), EGF (20 ng/ml), Ca (6 mM) and Pi (4 mM)]. Following this pre treatment the construct was implanted subcutaneously in the back at the cervical region of NMRI-nu/nu mice. Bone spicules (B′ and black arrow heads) were observed surrounding all CaP granules (GF/hPDC, left panel) a magnified area of this implant (defined by dashed box in left panel and shown in right) indicates the association of the growing bone with the CaP surface and also the presence of large quantities of bone lining cells (Inset) surrounding the de novo bone. The presence of fibrous tissue (FT) filled the remainder of the implant volume. In contrast treatment of hPDC seeded CPRM with GM for 10 days (GM/hPDC) resulted in the formation of only sporadic bone spicules, additionally these were not associated with the presence of bone lining cells (* Inset). Treatment of CPRM with the GF cocktail in the absence of cells (GF) did not result in the formation of any bone, however in encapsulation of the construct with a tissue rich in large blood filled vessels was observed (white arrows Inset). Fluorescence based histomorphometric Quantification of de novo bone in each condition revealed a 6 fold increase in bone formation following pretreatment of hPDCs with the GF cocktail when compared to the GM treated hPDC condition. (n=3; Statistical significance: ***: p<0.001 ANOVA; Scale bars: left panel=500 μm; right panel=200 μm; inset=50 μm; dashed boxes indicate areas of higher magnification)

FIG. 5: Validation of microarray gene expression with Sybr green PCR utilizing primers that recognize human specific transcripts for Anoctamin-1 (ANO1), Naked Cuticle (NKD2), Osterix (OSX), Osteopontin (OPN), Sarcolipin (SLN), and Bone Sialo Protein (BSP). Black bars: microarray expression, gray bars: expression measured with Sybr green PCR. Error bars: standard error of the mean (n=3 donors).

FIG. 6: Overview of temporal profiles for all individual gene clusters which are grouped into six superclusters (Solid line: average gene expression in CPRM, dashed line: average gene expression in CPDM).

FIG. 7: Western blot for p-pERK (MAPK signaling), p-p53, p-Smad 1/5/8 (BMP signaling), p-Smad 2 (TGFβ signaling), p-CREB (cAMP and EGF signaling), p-NFκB (TNFα/NFκB signaling), and p-β catenin (β-catenin/Wnt signaling). For each time point (day 2, 8 and 18 post implantation) protein expression was assessed in CPDM and CPRM for three different donors (d1, d2, d3).

FIG. 8: A) Identification of factors that drive proliferation of hPDCs. A cell pool of hPDCs was either treated with growth medium (GM, negative control), medium containing eight factors (all factors) or medium containing eight minus one factor for 8 days. The factors are osteogenic medium (OM), calcium ions (Ca, 6 mM), phosphate ions (Pi, 4 mM), TNFα (50 ng/ml), IL6 (10 ng/ml), Wnt3A (50 ng/ml), EGF (20 ng/ml), and TGFβ1 (10 ng/ml). Proliferation is expressed as population doublings and was measured after 8 days after stimulation (n=3, error bars: standard deviation). The horizontal line is a reference line set on the proliferation in the “all factor” condition. B) Identification of factors that drive alkaline phosphatase activity in hPDCs. The percentage alkaline positive cells is used as a metric for early osteoblast differentiation.

Same experimental design as in A. C) Identification of factors involved in osteoblast maturation. hPDCs were treated with OM and TGFβ1 for 6 days, followed by stimulation with GM (negative control), GM containing six factors or GM supplement with six minus one factor for 4 days. The factors are ascorbic acid (Asc. Ac., 57 μM), TNFα (50 ng/ml), IL6 (10 ng/ml), EGF (20 ng/ml), Ca (6 mM) and Pi (4 mM). To evaluate osteoblast maturation, gene expression of RUNX2, OSX, DLX5, iBSP, OC and RANKL is measured with Taqman PCR. Gene expression is normalized to GAPDH and displayed as 2^{-dcT} (n=3, error bars: standard deviation). D) Gene expression of osteoblast markers in hPDCs treated with OM and TGFβ1 for one week followed by GM supplemented with a growth factor mix (GF) containing ascorbic acid (57 μM), EGF (20 ng/ml), IL6 (10 ng/ml), Ca (6 mM), and Pi (4 mM) (“GF+C6P4”) or the same mix with reduced Ca (3 mM) and Pi (2 mM) ions (“GF+C3P2”). Gene expression is expressed as fold increase as compared to the GM condition. (n=3, error bars: standard deviations, *p≦00.05, Mann-Whitney U test). E) Gene expression of osteoblast markers in hPDCs treated with OM/TGFβ1 for 10 days, or with GM supplemented with ascorbic acid, EGF, IL6, C3P2 for 10 days (“EGF/IL6/C3P2”), or sequential stimulation with OM/TGFβ1 for 6 days followed by GM supplemented with ascorbic acid, EGF, IL6, C3P2 (“EGF/IL6/C3P2”) for 4 days. Gene expression is expressed as fold increase as compared to hPDCs cultured in OM/TGFβ1 (n=3, error bars: standard deviations, *p≦00.05, Mann-Whitney U test).

FIG. 9: Gene expression of early (A) and late (B) bone markers in hPDCs treated with GM, OM or GM/OM supplemented with a growth factor mix (GF) containing TNFα, EGF, TGFβ1 and IL6. C) ALP staining of hPDCs that were stimulated with OM and TGFβ1 for one week followed by GM with one factor in the absence or presence of ascorbic acid for 2 days. D) ALP/Alizarin Red staining to stain calcium deposits on hPDCs pre-treated with OM and TGFβ1 for one week followed by GM with six minus one factors for four days in the absence or presence of ascorbic acid.

FIG. 10: Potency of GFC on proliferation and osteogenic differentiation of hPDCs in 3D. A) Bright field images of hPDC/Collagen/fibrinogen microtissues, 24 h after seeding. B) Quantification of the number of EDU positive cells per microtissue [4≦n≦10 microtissues (except 6 posts GFC condition: n=1), bar=standard deviation]. C) Relative gene expression of bone markers in microtissues treated for 3 weeks in GM, OM or GFC. Gene expression is normalized to GM controls with exception of OPN gene expression. No OPN mRNA was detected in GM condition; hence gene expression of OPN in microtissues stimulated with OM and GFC is relative to the housekeeping gene GAPDH. (ND: not detected, OSX: osterix, Runx2: Runt-related transcription factor 2, Col1a2: collagen type I a2, BMP2: bone morphogenetic protein, OPN: osteopontin, BSP: Bone Sialo Protein, RANKL: Rank ligand, OCN: osteocalcin.)

### DETAILED DESCRIPTION OF THE INVENTION

One aspect of the invention relates to the methods developed by the inventors to produce cells with an osteogenic phenotype in vitro. Cell culture conditions were developed and optimized as described in detail in this invention (e.g. in the examples part). The inventors developed specific cell culture conditions to successfully proliferate and differentiate cells that express osteogenic phenotypes.

One embodiment of the present invention concerns a method for inducing cells to proliferate and differentiate into cells with an osteogenic phenotype. Certain embodiments of the present invention concern the growth factors and other components that are comprised in such a medium for said proliferation and differentiation of said cells. One embodiment of the present invention concerns an additional first incubation/culturing period with TNFα. Said TNFα can be added to the growth factor containing medium in said first incubation period or alternative said cells are first incubated in the presence of TNFα, without the extra growth factors (TGFβ, EGF, and IL6) of the present invention. Said first incubation period is meant to temporary inhibit differentiation of the cells, while allowing proliferation of the cells. In a specific embodiment, said first incubation period is maximum 4 days, or is 1, 2, or 3 days. In one embodiment said proliferation and differentiation period is at least four days, including 5, 6, 7, 8, 9, 10, 11, 12, 13, 14, 15, 16, 17, 18, 19, 20 and 21 days. In one embodiment said proliferation period is about 11 days. Thus also contemplated in the present invention is a total incubation period which is separated in an initial (mainly) proliferation step and a second (mainly) differentiation step. In such a first step, TNFα can be added to the growth medium (proliferation medium), in the presence or absence of other growth factors (such as TGFβ, EGF, and IL6), and in the second step TNFα is not present in the growth factor (TGFβ, EGF, and IL6) containing (mainly) differentiation step. Thus also contemplated in the present invention is a method comprising a first mainly differentiation step as described hereabove and a second mainly differentiation step as described hereabove for inducing cells to proliferate and differentiate into cells with an osteogenic phenotype. In certain embodiments of the present invention, in said first proliferation step cells are cultured for 1, 2, 3, or 4 days and in said second step the cells are further incubated for 1, 2, 3, 4, 5, 6, 7, 8, 9, 10, 11, 12, 13, 14, 15, 16, 17, 18, 19, or 20 days. Thus a combination of a 4 days step 1 and a 7 days step 2 and the like combinations are also contemplated in the present invention.

Further embodiments of the present invention concern the addition of other factors in the growth factor containing culture medium of the present invention. Said other factors are at least one factor selected from the group consisting of: Retinoic acid, hepatocyte nuclear factor 4A, Amyloid beta (A4) precursor protein, beta-estadriol, and interferon gamma.

One embodiment of the present invention concerns a method for inducing cells to proliferate and differentiate into cells with an osteogenic phenotype, comprising:
(a) obtaining cells from a biological sample
(b) expanding said cells in a first proliferation step in proliferation medium
(c) differentiating said cells in a second differentiation step in the growth factor containing medium of the present invention;
wherein step (b) and step (c) can be sequential or simultaneous in time.

One embodiment of the present invention concerns the proliferation and or differentiation culturing step being performed in a culture dish or plate or in a 3-D culturing facilitating incubation step, wherein the cells are optionally co-cultured with non-cellular or scaffold material. In a more detailed embodiment, such co-culture from cells with scaffold material results in the formation of an implantable graft.

In certain embodiments of the present invention in said culturing steps the cells are cultured until passage number 6, 7, 8 or 9. In other embodiments of the present invention said cells to be cultured are seeded at a cell density of about 2000 to about 4000 cells/cm², in more preferred embodiments said density is about 3000 cells/cm².

One embodiment of the present invention concerns the cells, wherein the cells are stem cells, more preferably mesenchymal cells, such as periosteum derived cells. In a preferred embodiment, said cells are of mammalian in particular human origin.

One embodiment of the present invention concerns a method of treatment comprising administering a therapeutically effective amount of the cells produced according to any one of the methods of this invention to a subject with a bone disorder, said bone disorder includes a bone fracture. A preferred embodiment of the present invention relates to said method of treatment to treat a subject, preferably a human, with a non-healing bone defect.

Alternatively, the present invention concerns the use of cells produced according to any one of the methods of this invention or a pharmaceutical composition according to the present invention for use in medicine, more particularly for use in the treatment of a subject with a bone disorder.

One embodiment of the present invention relates to said use or method of treatment wherein the cells produced by the methods of this invention are injected in the bone defects of said subject. In certain embodiments of the present invention said use or method of treatment comprises the injection of the cells of the present invention that are produced at an intermediate timepoint of the methods of this invention, such as the endpoint of the proliferation step and wherein said intermediate cells are injected in the subject together with the growth factor containing medium of the present invention. In certain embodiments of the hereabove described uses or methods of treatment, such (intermediate) cells can be administered to said subject with the growth factor containing medium in combination with a scaffold or non-cellular material, which can optionally be pre-incubated in vitro, before administration to said subject. One embodiment of the present invention relates to said uses or treatment of the present invention with optionally further administration of other cells such as stem cells, endothelial cells, or haematopoetic (progenitor) cells. Such further administration of other cells can be simultaneously or sequentially in time with the cells of the present invention. In one embodiment, such other cells, such as endothelial cells, are cocultured with the cells of the present invention, before administration to said subject or patient. In another embodiment such other cells, such as endothelial cells, are cultured separately from the cells of the present invention, and are mixed together at the time of the administration to said subject or patient. In one embodiment said cells of the present invention, optionally with said other cells (eg. endothelial cells) are pre-cultured with other non-cellular material, biomaterial, or scaffolds for optimal treatment, such as an optimal bone forming effect in said subject or patient. In other embodiments of the present invention, said cells of the present invention, optionally with said other cells (eg. endothelial cells) are mixed together with other non-cellular material, biomaterial, or scaffolds at the time of the administration to said subject or patient.

In certain preferred embodiments, said subject is a human, more particularly a human with a bone defect, more particularly a non-healing bone defect.

One embodiment of the present invention concerns the immobilization of components of the growth factor medium by use of a biomaterial before administration to said patient, with the purpose to simultaneous or sequential release of the factors in said subject or patient.

One embodiment of the present invention concerns the delivery of the components of the Growth Factor Medium, of the present invention, by engineering cells to synthesize and secrete said components before administration to said subject or patient. Such engineered cells can be administered to said subject or patient optionally in combination with non-cellular material, biomaterial or scaffold material, and optionally together with other cells, such as stem cells, endothelial cells, or haematopoetic (progenitor) cells.

In one embodiment, osteoblast progeny can be used to ameliorate a process having deleterious effects on bone including, but not limited to, bone fractures, non-healing fractures, osteoarthritis, “holes” in bones cause by tumors spreading to bone such as prostate, breast, multiple myeloma, and the like.

In one embodiment, the present invention provides a screening method in which the differentiated cells with an osteogenic phenotype are used to characterize cellular responses to biologic or pharmacologic agents involving contacting the cells with one or more biologic or pharmacologic agents. Such agents may have various activities. They could affect differentiation, metabolism, gene expression, viability and the like. The cells are useful, therefore, for e.g. toxicity testing and identifying differentiation factors.

In one embodiment, the differentiated cells can be used to study the effects of specific genetic alterations, toxic substances, chemotherapeutic agents, or other agents on the developmental pathways. Tissue culture techniques known to those of skill in the art allow mass culture of hundreds of thousands of cell samples from different individuals, providing an opportunity to perform rapid screening of compounds suspected to be, for example teratogenic or mutagenic.

In one embodiment, the differentiated cells can also be genetically engineered, by the introduction of foreign DNA or by silencing or excising genomic DNA, to produce differentiated cells with a defective phenotype in order to test the effectiveness of potential chemotherapeutic agents or gene therapy vectors.

Cell Culture.

In general, cells useful for the invention can be maintained and expanded in growth or culture medium that is available to and well-known in the art. Such media include, but are not limited to, Dulbecco's Modified Eagle's Medium® (DMEM), DMEM F12 medium®, Eagle's Minimum Essential Medium®, F-12K medium®, Iscove's Modified Dulbecco's Medium® and RPMI-1640 medium®. Many media are also available as low-glucose formulations, with or without sodium pyruvate.

Also contemplated in the present invention is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements and bovine embryonic fluid or platelet rich plasma (PRP). It is understood that sera can be heat-inactivated at 55-65° C. if deemed necessary to inactivate components of the complement cascade.

Additional supplements, in addition to the growth factors and other factors described in the present invention, also can be used advantageously to supply the cells with the necessary trace elements for optimal growth and expansion. Such supplements include insulin, transferrin, sodium selenium and combinations thereof. These components can be included in a salt solution such as, but not limited to, Hanks' Balanced Salt Solution®

(HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Cells may be cultured in low-serum or serum-free culture medium. Many cells have been grown in serum-free or low-serum medium. In this case, the medium is supplemented with one or more growth factors. Commonly used growth factors include, but are not limited to, bone morphogenic protein, basis fibroblast growth factor, platelet-derived growth factor and epidermal growth factor. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium.

In one embodiment of the present invention, the cells may be cultured in the presence of antibiotics, such as Pennicilin/streptomycin, eg in an antibiotics concentration of 1%.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007).

Cells may also be grown in “3D” (aggregated) cultures as described in WO2009092092 or in 3D microtissues as examplified in Example 3.

Once established in culture, cells can be used fresh or frozen and stored as frozen stocks, using, for example, DMEM with 40% FCS and 10% DMSO. Other methods for preparing frozen stocks for cultured cells also are available to those skilled in the art.

Methods of identifying and subsequently separating differentiated cells from their undifferentiated counterparts can be carried out by methods well known in the art. Cells that have been induced to differentiate using methods of the present invention can be identified by selectively culturing cells under conditions whereby differentiated cells outnumber undifferentiated cells. Similarly, differentiated cells can be identified by morphological changes and characteristics that are not present on their undifferentiated counterparts, such as cell size and the complexity of intracellular organelle distribution. Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS) and enzyme-linked immunosorbent assay (ELISA). From the standpoint of transcriptional upregulation of specific genes, differentiated cells often display levels of gene expression that are different from undifferentiated cells. Reverse-transcription polymerase chain reaction, or RT-PCR, also can be used to monitor changes in gene expression in response to differentiation. Whole genome analysis using microarray technology also can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads and combinations thereof. One embodiment of the present invention comtemplates the use of FACS to identify and separate cells based on cell-surface antigen expression.

Pharmaceutical Formulations.

Any of the cells produced by the methods described herein can be used in the clinic to treat a subject. They can, therefore, be formulated into a pharmaceutical composition. Therefore, in certain embodiments, the isolated or purified cell populations are present within a composition adapted for and suitable for delivery, i.e., physiologically compatible. Accordingly, compositions of the cell populations will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

In other embodiments, the isolated or purified cell populations are present within a composition adapted for or suitable for freezing or storage.

In many embodiments the purity of the cells for administration to a subject is about 100%. In other embodiments it is 95% to 100%. In some embodiments it is 85% to 95%. Particularly in the case of admixtures with other cells, such as endothelial cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 5-10, 10-20, 20-30, 30-40, 40-50 or more cell doublings.

The numbers of cells in a given volume can be determined by well known and routine procedures and instrumentation. The percentage of the cells in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

The choice of formulation for administering the cells for a given application will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of administration and the nature of the particular dosage form.

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic cells. Various embodiments of the invention comprise measures to increase cell survival and/or to overcome problems posed by barriers to seeding and/or growth.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant, such as maltose, which must be tolerated by the body. Exemplary lubricant components include glycerol, glycogen, maltose and the like. Organic polymer base materials, such as polyethylene glycol and hyaluronic acid as well as non-fibrillar collagen, preferably succinylated collagen, can also act as lubricants. Such lubricants are generally used to improve the injectability, intrudability and dispersion of the injected biomaterial at the site of injection and to decrease the amount of spiking by modifying the viscosity of the compositions. This final formulation is by definition the cells in a pharmaceutically acceptable carrier.

The cells are subsequently placed in a syringe or other injection apparatus for precise placement at the site of the tissue defect. The term “injectable” means the formulation can be dispensed from syringes having a gauge as low as 25 under normal conditions under normal pressure without substantial spiking. Spiking can cause the composition to ooze from the syringe rather than be injected into the tissue. For this precise placement, needles as fine as 27 gauge (200μ I.D.) or even 30 gauge (150μ I.D.) are desirable. The maximum particle size that can be extruded through such needles will be a complex function of at least the following: particle maximum dimension, particle aspect ratio (length:width), particle rigidity, surface roughness of particles and related factors affecting particle:particle adhesion, the viscoelastic properties of the suspending fluid, and the rate of flow through the needle. Rigid spherical beads suspended in a Newtonian fluid represent the simplest case, while fibrous or branched particles in a viscoelastic fluid are likely to be more complex.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or stabilizer can be employed to increase the life of cell/medium compositions. If such preservatives are included, it is well within the purview of the skilled artisan to select compositions that will not affect the viability or efficacy of the cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells/medium utilized in practicing the present invention in the required amount of the appropriate solvent with various amounts of the other ingredients, as desired.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments cells are encapsulated for administration, particularly where encapsulation enhances the effectiveness of the therapy, or provides advantages in handling and/or shelf life. Encapsulation in some embodiments where it increases the efficacy of cell mediated immunosuppression may, as a result, also reduce the need for immunosuppressive drug therapy.

Also, encapsulation in some embodiments provides a barrier to a subject's immune system that may further reduce a subject's immune response to the cells (which generally are not immunogenic or are only weakly immunogenic in allogeneic transplants), thereby reducing any graft rejection or inflammation that might occur upon administration of the cells.

Cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

Dosing.

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of cells/medium appropriate to be used in accordance with various embodiments of the invention will depend on numerous factors. It may vary considerably for different circumstances. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration. Finally, the determination of optimal dosing necessarily will provide an effective dose that is neither below the threshold of maximal beneficial effect nor above the threshold where the deleterious effects associated with the dose outweighs the advantages of the increased dose.

It is to be appreciated that a single dose may be delivered all at once, fractionally, or continuously over a period of time. The entire dose also may be delivered to a single location or spread fractionally over several locations.

In various embodiments, cells/medium may be administered in an initial dose, and thereafter maintained by further administration. Cells/medium may be administered by one method initially, and thereafter administered by the same method or one or more different methods. The levels can be maintained by the ongoing administration of the cells/medium. Various embodiments administer the cells/medium either initially or to maintain their level or expand in the subject. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regimens can be ascertained by the skilled artisan, from this disclosure, the documents cited herein, and the knowledge in the art.

The dose, frequency, and duration of treatment will depend on many factors, including the nature of the disorder, the subject, and other therapies that may be administered. Accordingly, a wide variety of regimens may be used to administer the cells/medium.

In some embodiments cells/medium are administered to a subject in one dose. In others cells/medium are administered to a subject in a series of two or more doses in succession. In some other embodiments wherein cells/medium are administered in a single dose, in two doses, and/or more than two doses, the doses may be the same or different, and they are administered with equal or with unequal intervals between them.

Cells/medium may be administered in many frequencies over a wide range of times. In some embodiments, they are administered over a period of less than one day. In other embodiment they are administered over two, three, four, five, or six days. In some embodiments they are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

Definitions:

As used herein and unless otherwise stated, the term “growth factor medium” means a combination of growth medium and a growth factor cocktail. The growth medium contains DM EM cell culture medium, 10% fetal bovine serum and 1% penicillin/streptomycin. The growth factor cocktail contains 20 ng/ml EGF, 10 ng/ml IL6, 10 ng/ml TGFβ1, 50 μM ascorbic acid, 3 mM calcium ions in HBS buffer, and 2 mM phosphate ions in HBS buffer. The composition of the growth factor medium is described in example 2, table 6.

The concentration of TGFβ1 that is added to the growth factor containing medium can range from about 1 ng/ml to about 100 ng/ml TGFβ1. However, the invention also emcompasses sub-ranges of concentrations of TGFβ1. For example, from about 1-10 ng/ml, 1-20 ng/ml, 1-30 ng/ml, 1-40 ng/ml, 1-50 ng/ml, 1-60 ng/ml, 1-70 ng/ml, 1-80 ng/ml and 1-90 ng/ml. The preferred concentration of TGFβ1 that is added to the growth factor containing medium is 10 ng/ml.

The concentration of EGF that is added to the growth factor containing medium can range from about 2 ng/ml to about 200 ng/ml EGF. However, the invention also emcompasses sub-ranges of concentrations of EGF. For example, from about 2-20 ng/ml, 2-30 ng/ml, 2-40 ng/ml, 2-50 ng/ml, 2-60 ng/ml, 2-70 ng/ml, 2-80 ng/ml, 2-90 ng/ml, 2-100 ng/ml, 2-110 ng/ml, 2-120 ng/ml, 2-130 ng/ml, 2-140 ng/ml, 2-150 ng/ml, 2-160 ng/ml, 2-170 ng/ml, 2-180 ng/ml and 2-190 ng/ml. The preferred concentration of EGF that is added to the growth factor containing medium is 20 ng/ml.

The concentration of IL6 that is added to the growth factor containing medium can range from about 1 ng/ml to about 100 ng/ml IL6. However, the invention also emcompasses sub-ranges of concentrations of IL6. For example, from about 1-10 ng/ml, 1-20 ng/ml, 1-30 ng/ml, 1-40 ng/ml, 1-50 ng/ml, 1-60 ng/ml, 1-70 ng/ml, 1-80 ng/ml and 1-90 ng/ml. The preferred concentration of IL6 that is added to the growth factor containing medium is 10 ng/ml.

The concentration of calcium ions that is added to the growth factor containing medium can range from about 0.3 mM to about 12 mM. However, the invention also emcompasses sub-ranges of concentrations of calcium ions. For example, from about 0.3-5 mM, 3-5 mM, 0.3-7 mM, 3-7 mM, 0.3-9 mM, 3-9 mM and 3-12 mM. The preferred concentration of calcium ions that is added to the growth factor containing medium is 3 mM.

The concentration of serum that is added to the growth factor containing medium can range from about 0% to about 20%. However, the invention also emcompasses sub-ranges of concentrations of serum. For example, from about 0-10%, 5-10%, 5-15%, 10-15%, 5-20% and 10-20%. The preferred concentration of serum that is added to the growth factor containing medium is 10%.

The concentration of ascorbic acid that is added to the growth factor containing medium can range from about 10⁻⁴NA to about 10⁻⁷M. However, the invention also emcompasses sub-ranges of concentrations of ascorbic acid. For example, from about 10⁻⁴-10⁻⁵M, 10⁻⁴-10⁻⁶M, 10⁻⁴-10⁻⁷M, 5×10⁻⁵-10⁻⁶M and 5×10⁻⁵-10⁻⁷M. The preferred concentration of ascorbic acid that is added to the growth factor containing medium is 50 μM.

The concentration of phosphate ions that is added to the growth factor containing medium can range from about 0.2 mM to about 8 mM. However, the invention also emcompasses sub-ranges of concentrations of phosphate ions. For example, from about 0.2-4 mM, 2-4 mM, 0.2-6 mM, 2-6 mM and 2-8 mM. The preferred concentration of calcium ions that is added to the growth factor containing medium is 2 mM.

As used herein and unless otherwise stated, the term “ osteogenic phenotype ” means expression of gene markers, that are well known to a person skilled in the art, such as alkaline phosphatase, collagen type I, osterix, osteocalcin, cadherin 11, RANK ligand,

BMP2, Bone Sialo Protein and Secreted Phospho Protein 1 and is able to form bone tissue when implanted in an orthotopic, heterotopic or ectopic environment in vivo as well known to a person skilled in the art.

As used herein and unless otherwise stated, the term “ mesenchymal cells ” means any cell type derived from tissues originating from the mesoderm or neural crest during embryonic development or have the phenotype as described in Dominici et al. (Dominici 2006, Cytotherapy, Vol.8 n° 4, 315-17).

As used herein and unless otherwise stated, the term “ periosteum derived cells ” means any cell type that is isolated from the periosteum well known to a person skilled in the art.

As used herein and unless otherwise stated, the term “ cells that express a primitive mesenchymal phenotype ” means any cell type originating from the mesoderm or neural crest during embryonic development or derived from stem cell differentiation or (partial) dedifferentiation such as by the IPS technology, well known to the skilled person, and which will give rise to cells that contribute to all mesenchymal tissues as known to a person skilled in the art. These primitive cells may express markers that upon genetic labeling at the moment of expression, can be found in any mesenchymal tissue at later stages of development. Examples of such markers include but are not limited to PRX1, PRX2, and Sox9.

As used herein and unless otherwise stated, the term “ bone disorders ” means any medical condition that affects the bone, examples of such bone disorders include but are not limited to bone diseases such as osteoporosis, Paget's disease, congenital pseudoarthrosis, among others and also include bone injuries such as bone fractures, delayed union fractures and non-healing bone disorders as known to a person skilled in the art.

As used herein and unless otherwise stated, the term “ non-healing bone defect” means permanent failing of healing of a structural defect of the bone leading to loss of integrity. Examples of such non union bone defects include but are not limited to atrophic, hypertrophic fractures and large bone defects as known to a person skilled in the art.

“ Stem cell ” means a cell that can undergo self-renewal (i.e., progeny with the same differentiation potential) and also produce progeny cells that are more restricted in differentiation potential. Within the context of the invention, a stem cell would also encompass a more differentiated cell that has dedifferentiated, for example, by nuclear transfer, by fusions with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass).

“ Subject ” means a vertebrate, such as a mammal. Mammals include, but are not limited to, humans, dogs, cats, horses, cows and pigs.

The term “ therapeutically effective amount ” refers to the amount determined to produce any therapeutic response in a mammal. For example, effective amounts of the therapeutic cells or cell-associated agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are ascertained by one of ordinary skill in the art through routine application to subject populations such as in clinical and pre-clinical trials. Thus, to “treat” means to deliver such an amount. “Treat”, “treating” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

The present invention is additionally described by way of the following illustrative, non-limiting Examples providing a better understanding of the present invention and of its many advantages.

### EXAMPLES

### Example 1: Experimental Procedures

Cell culture. Periosteum was harvested from four patients (male/female/age) and periosteal cells were enzymatically released from the matrix. Tissue culture plastic adherent cells were expanded in DMEM medium supplemented with 10% fetal bovine serum as described previously (6). For in vitro osteogenic differentiation assays, passage 6 to passage 9 hPDCs (pool of four different donors) were seeded at 3000 cells/cm² in either 96-well plates to assess proliferation and alkaline phosphatase activity or in the middle eight wells of a 24-well plate for quantifying gene expression.

Medium was changed every other day. Supplemental factors were TNFα, IL6 (R&D Systems, USA), TGFβ1 (Stem Cell Research, USA), Ascorbic Acid (Sigma,USA), Ca and Pi (SigmaUSA). Calcium and phosphate ion working solutions were prepared as described in (7).

Preparation of the scaffolds. Collagraft™ (Neucoll Inc., Cambell, Calif., US), an open porous composite made of calcium phosphate (CaP) granules consisting of 65% hydroxyapatite (HA) and 35% β-tri-calcium phosphate (β-TCP), embedded in a bovine collagen type I matrix, was punched into 21 mm³ cylindrical (diameter 3 mm, height 3 mm) scaffolds. Half of the Collagraft™ carriers were immersed in an EDTA/PBS buffer for two weeks to reduce the amount of calcium phosphate. Control scaffolds were left untreated. After treatment, the scaffolds were washed twice with PBS followed by lyophilization to dry the structures.

In vivo osteogenesis. Passage 3 hPDCs were trypsin released, centrifuged and re-suspended at a concentration of 20 million cells/ml. One million cells were drop seeded on the upper surface of each scaffold (Collagraft™ or EDTA decalcified Collagraft™) or replated in a T175 flask (2D reference condition) and incubated overnight at 37° C. to allow cell attachment. After incubation, the Collagraft™ was directly implanted subcutaneously in the back at the cervical region of NMRI-nu/nu mice. All procedures on animal experiments were approved by the local ethical committee for Animal Research (Katholieke Universiteit Leuven). The animals were housed according to the guidelines of the Animalium Leuven (Katholieke Universiteit Leuven).

RNA extraction and microarray analysis. Twenty hours after seeding (in vitro) and 2, 8 and 18 days after implantation (in vivo) implants were harvested, flash frozen in liquid nitrogen, homogenized (Ingenieurburo CAT M. Zipperer GmbH, Staufen, Germany) and processed for RNA extraction with the fibrous mini RNA extraction kit (Qiagen) according to the manufacturer's procedures. The microarrays were processed by the Micro Array Facility of the VIB (Flemish Institute of Biotechnology, Leuven, Belgium). Briefly, one microgram of RNA from each sample that passed the Quality Control as determined by band densitometry of ribosomal RNA was spotted on Agilent Single Color

Human MicroArray Chips (Agilent H44K). Fluorescent intensities were measured and converted into Log2 values. Differentially gene expression between two consecutive time points or between the Collagraft™ and decalcified Collagraft™ condition was determined by student t-test with a cut off p-value of 0.001.

Selection of Gene Of Interest (GOI) and bioinformatics analysis. A GOI was defined as a gene which was differentially expressed between two consecutive time points in the Collagraft™ condition, but not in the decalcified condition and which was differentially expressed between the two conditions at the latter time point (cut-off: p<0.001). After removing duplicate probes and unknown ID's the list of GOI contained 946 genes (Table 2).

TABLE 2
List of GOI: genes that are significantly regulated between two consecutive
time points in CPRM and differentially expressed as compared to CPDM. For each time
point, genes are ranked from high to low expression (italic). Values are log ratios
normalized to gene expression levels of plastic adherent cells at 20 h after seeding.
Genes marked in bold are genes which are associated with bone formation according to
gene annotation in DAVID.

| | | Log2 expression levels | | | | | | | |
|---|---|---|---|---|---|---|---|---|---|
| | | EDTA decalcified | | | | | | | |
| | | Collagraft | | | | Collagraft | | | |
| Gene Name | Pubmed ID | 20 h | 2 days | 8 days | 18 days | 20 h | 2 days | 8 days | 18 days |

Up regulated and differentially expressed at 20 h

| | | | | | | | | | |
|---|---|---|---|---|---|---|---|---|---|
| NR4A3 | NM_173199 | 2.46 | 0.13 | 0.06 | 0.01 | 5.01 | 0.63 | 0.12 | 0.00 |
| NR4A1 | NM_002135 | 2.70 | 1.54 | 1.17 | 0.26 | 4.47 | 2.33 | 2.21 | 0.48 |
| BCL2L11 | NM_138621 | 2.24 | 5.62 | 5.88 | 5.66 | 3.9/ | 5.77 | 5.82 | 5.47 |
| SOCS2 | NM_003877 | 1.52 | 1.07 | 0.41 | 0.42 | 2.61 | 1.31 | 0.51 | -0.20 |
| KIAA0513 | NM_014732 | −0.05 | 1.75 | 1.78 | 1.48 | 2.46 | 2.41 | 2.34 | 0.52 |
| PTPN1 | NM_002827 | 0.49 | −0.54 | −1.16 | −1.27 | 2.39 | −0.80 | −1.20 | −0.74 |
| LOC730167 | XM_001134097 | 0.39 | −0.01 | 1.18 | −0.44 | 2.36 | 0.82 | 1.78 | −1.24 |
| GNB5 | BC011671 | 0.22 | −1.33 | −1.38 | −1.29 | 2.31 | −1.24 | −1.30 | −0.94 |
| LRRC17 | NM_005824 | 3.19 | 5.47 | 8.32 | 7.79 | 2.11 | 6.91 | 8.60 | 8.33 |
| PTP4A1 | NM_003463 | 0.62 | 0.05 | 0.33 | 0.38 | 2.07 | 0.51 | 0.66 | 0.12 |
| SETX | NM_015046 | 0.27 | −0.03 | −0.07 | −0.15 | 1.41 | −0.15 | −0.01 | −0.10 |
| C1ORF88 | NM_181643 | 2.63 | 0.96 | 0.56 | 1.28 | 1.38 | 1.91 | 0.16 | 0.70 |
| MUC13 | NM_033049 | 0.19 | 0.23 | 0.07 | −0.07 | 1.32 | 0.18 | 0.34 | −0.09 |
| ZBTB10 | NM_023929 | −0.01 | −0.46 | −0.50 | −0.27 | 1.07 | −0.37 | −0.46 | −0.14 |

Down regulated and differentially expressed at 20 h

| | | | | | | | | | |
|---|---|---|---|---|---|---|---|---|---|
| TFDP3 | NM_016521 | −2.46 | −3.37 | −3.39 | −3.44 | −1.45 | −3.37 | −3.37 | −3.32 |
| CCNJ | NM_019084 | −2.43 | 0.72 | 1.24 | 1.85 | −1.21 | 1.38 | 1.95 | 1.41 |

Up regulated and differentially expressed at 2 days

| | | | | | | | | | |
|---|---|---|---|---|---|---|---|---|---|
| SORBS1 | NM_006434 | 0.02 | 4.43 | 8.84 | 8.99 | 0.02 | 9.24 | 9.11 | 8.12 |
| C1QB | NM_000491 | 0.00 | 7.26 | 10.17 | 10.38 | 0.00 | 8.76 | 10.23 | 9.48 |
| CCND2 | NM_001759 | 0.91 | 5.75 | 8.29 | 8.25 | 0.92 | 7.81 | 8.27 | 7.58 |
| C4B | NM_001002029 | 0.78 | 4.61 | 8.74 | 9.30 | 0.74 | 7.43 | 9.21 | 8.56 |
| LOC100292101 | BC048193 | 1.52 | 3.81 | 6.72 | 5.95 | 1.72 | 7.43 | 6.31 | 4.92 |
| RBP1 | NM_002899 | 0.00 | 4.62 | 9.16 | 9.43 | −0.01 | 7.07 | 9.96 | 9.23 |
| LRRC17 | NM_005824 | 3.19 | 5.47 | 8.32 | 7.79 | 2.11 | 6.91 | 8.60 | 8.33 |
| TPPP3 | NM_016140 | 0.36 | 5.38 | 6.35 | 6.49 | 0.31 | 6.76 | 7.14 | 6.09 |
| PTPRD | NM_002839 | −0.13 | 3.11 | 7.64 | 8.33 | 0.42 | 6.45 | 8.12 | 8.07 |
| GPD1 | NM_005276 | −0.14 | −0.25 | 6.19 | 5.77 | 0.23 | 6.42 | 5.48 | 5.60 |
| TIE1 | NM_005424 | 0.01 | 2.00 | 6.97 | 6.89 | −0.28 | 6.33 | 7.20 | 6.61 |
| CPXM1 | NM_019609 | 0.55 | 4.31 | 7.63 | 8.38 | 0.54 | 6.26 | 8.13 | 6.61 |
| HIST1H2AB | NM_003513 | −0.16 | 4.95 | 6.38 | 6.91 | 0.03 | 6.10 | 6.62 | 6.29 |
| SPTB | NM_001024858 | −0.25 | 2.04 | 3.91 | 3.98 | −0.72 | 6.07 | 5.55 | 3.65 |
| PCDH19 | NM_020766 | 0.48 | 3.92 | 6.83 | 6.74 | 0.40 | 6.02 | 7.38 | 5.70 |
| ITM2A | NM_004867 | −0.31 | 2.84 | 8.75 | 8.39 | −0.45 | 5.97 | 9.52 | 7.82 |
| TSPAN13 | NM_014399 | 0.14 | 4.20 | 6.58 | 6.74 | 0.01 | 5.97 | 6.59 | 5.70 |
| CPA3 | NM_001870 | −0.72 | 3.43 | 7.30 | 5.00 | −0.64 | 5.89 | 7.75 | 3.30 |
| DCAF4L2 | NM_152418 | 0.04 | 3.96 | 4.42 | 6.65 | 0.02 | 5.87 | 5.38 | 5.17 |
| SLC24A3 | NM_020689 | 0.52 | 3.12 | 6.17 | 6.72 | 0.68 | 5.77 | 6.76 | 5.98 |
| PPARGC1B | AK024346 | −0.17 | 3.81 | 6.11 | 6.88 | −0.17 | 5.68 | 6.96 | 7.33 |
| TSPAN7 | NM_004615 | 0.03 | 0.94 | 5.15 | 5.80 | −0.12 | 5.58 | 5.74 | 6.57 |
| S100A1 | NM_006271 | 0.06 | 3.58 | 6.61 | 7.58 | −0.10 | 5.52 | 6.55 | 6.41 |
| D4S234E | NM_014392 | −0.15 | 3.68 | 5.23 | 5.25 | −0.38 | 5.50 | 6.40 | 5.15 |
| LBP | NM_004139 | −0.01 | 2.91 | 6.11 | 6.07 | −0.02 | [symbol] | 7.67 | 5.58 |
| CTNNB1 | NM_001904 | −0.25 | 4.40 | 5.95 | 5.92 | −0.58 | [symbol] | 6.41 | 5.59 |
| PTH1R | NM_000316 | 0.88 | 2.32 | 6.58 | 6.67 | 0.47 | [symbol] | 6.72 | 7.35 |
| ALDH1L1 | NM_012190 | −0.45 | −0.12 | 5.08 | 5.20 | −0.43 | 5.43 | 4.88 | 4.39 |
| LMO1 | NM_002315 | 0.02 | 3.03 | 5.97 | 5.87 | 0.11 | 5.40 | 5.67 | 4.89 |
| HRASLS2 | NM_017878 | 0.22 | 2.99 | 6.19 | 5.67 | −0.15 | 5.28 | 6.78 | 4.90 |
| UBL4A | NM_014235 | −0.37 | 4.21 | 4.95 | 5.88 | −0.41 | 5.22 | 5.61 | 4.99 |
| GPC3 | NM_004484 | −0.95 | 2.86 | 7.10 | 7.00 | −1.04 | 5.19 | 7.52 | 5.95 |
| ENO3 | NM_001976 | 0.66 | 1.99 | 2.58 | 3.85 | 0.41 | 5.12 | 4.25 | 3.39 |
| BOK | AF089746 | −0.25 | 3.09 | 5.51 | 5.85 | 0.02 | 4.95 | 6.04 | 5.29 |
| HFE2 | NM_213653 | 0.02 | 1.07 | 0.98 | 0.27 | −0.14 | 4.84 | 3.16 | −0.13 |
| OPCML | NM_001012393 | −0.21 | 2.56 | 7.14 | 7.28 | −0.55 | 4.80 | 7.11 | 6.23 |
| MYL4 | NM_002476 | −0.28 | 1.66 | 4.71 | 4.58 | −0.62 | 4.80 | 5.99 | 3.91 |
| FBXL16 | NM_153350 | −0.13 | 2.85 | 5.08 | 5.63 | 0.21 | 4.69 | 5.92 | 5.70 |
| UNC45B | NM_173167 | −0.92 | −0.92 | 5.05 | 5.56 | −0.92 | 4.59 | 5.86 | 4.20 |
| MRPL2 | NM_015950 | 0.72 | 3.52 | 4.53 | 4.36 | 0.67 | 4.59 | 4.98 | 3.66 |
| RAD51L1 | NM_133510 | 0.79 | 2.22 | 6.99 | 5.82 | 0.53 | 4.53 | 7.19 | 4.00 |
| DMD | NM_004010 | −0.65 | 0.33 | 4.96 | 5.54 | −0.52 | 4.45 | 5.82 | 4.82 |
| PAX6 | NM_001604 | −0.03 | 2.81 | 3.43 | 7.09 | −0.04 | 4.42 | 4.63 | 5.57 |
| HIST2H4B | NM_003548 | 1.83 | 3.30 | 4.39 | 4.78 | 0.85 | 4.33 | 4.78 | 4.29 |
| MARK1 | NM_018650 | −0.45 | 2.07 | 5.30 | 6.27 | −0.36 | 4.30 | 6.16 | 4.88 |
| DACH1 | NM_080759 | 0.03 | 1.52 | 5.01 | 5.19 | −0.01 | 4.30 | 5.97 | 4.20 |
| DYSF | NM_003494 | 1.83 | 0.97 | 4.62 | 5.03 | 1.75 | 4.30 | 5.20 | 5.05 |
| PTPRB | NM_002837 | −0.12 | −0.61 | 4.78 | 4.61 | −0.19 | 4.21 | 5.12 | 3.94 |
| ABCG2 | NM_004827 | −0.15 | 2.46 | 4.98 | 5.06 | −0.38 | 4.20 | 5.29 | 4.25 |
| SHANK3 | NM_001080420 | 0.80 | 1.66 | 4.30 | 5.45 | 0.89 | 4.17 | 4.81 | 4.86 |
| ICA1 | NM_004968 | 0.76 | 1.46 | 5.24 | 5.73 | 0.50 | 4.14 | 5.64 | 5.27 |
| HIST1H4I | NM_003495 | 1.54 | 3.00 | 4.19 | 4.43 | 0.69 | 4.07 | 4.55 | 3.90 |
| MYLK2 | NM_033118 | −0.29 | 1.20 | 2.24 | 2.90 | −0.17 | 4.07 | 3.80 | 2.38 |
| HIST1H2AH | NM_080596 | −0.07 | 2.89 | 3.88 | 4.36 | 0.07 | 4.02 | 4.19 | 3.95 |
| HIST1H2AJ | NM_021066 | 0.13 | 2.90 | 4.13 | 4.52 | 0.16 | 4.00 | 4.37 | 3.97 |
| MKL2 | NM_014048 | −0.36 | 2.89 | 4.74 | 5.24 | −0.51 | 4.00 | 5.04 | 4.27 |
| HIST1H2AE | NM_021052 | 0.98 | 2.71 | 3.92 | 3.15 | 0.80 | 3.92 | 4.31 | 2.39 |
| HIST1H4F | NM_003540 | 1.60 | 2.87 | 4.16 | 4.35 | 0.68 | 3.92 | 4.52 | 3.82 |
| FFAR2 | NM_005306 | −0.21 | −0.71 | 3.23 | 2.20 | −0.09 | 3.91 | 1.82 | 1.02 |
| ASH1L | NM_018489 | −0.19 | 2.83 | 3.94 | 4.24 | −0.07 | 3.84 | 4.30 | 3.63 |
| HPR | NM_020995 | −0.01 | 0.58 | 1.77 | 0.36 | 1.00 | 3.84 | 0.42 | 0.13 |
| TEKT2 | NM_014466 | 1.47 | 2.20 | 3.91 | 4.49 | 0.91 | 3.81 | 4.40 | 4.16 |
| PVT1 | NR_003367 | 0.08 | 2.69 | 4.13 | 4.35 | 0.31 | 3.81 | 4.75 | 3.20 |
| LIPE | NM_005357 | 0.44 | 0.96 | 3.60 | 3.22 | 0.51 | 3.80 | 3.46 | 2.53 |
| HP | NM_005143 | 0.08 | 1.63 | 2.89 | 2.82 | 0.13 | 3.78 | 3.35 | 1.64 |
| ENTPD8 | NM_001033113 | −0.28 | 2.24 | 4.61 | 3.72 | −0.31 | 3.76 | 5.33 | 2.69 |
| GINS1 | NM_021067 | −0.87 | 1.75 | 1.82 | 0.97 | −0.75 | 3.74 | 3.80 | 0.97 |
| RHOJ | NM_020663 | −0.68 | 2.00 | 4.34 | 5.05 | −0.77 | 3.7/ | 4.79 | 4.09 |
| HIST1H2AK | NM_003510 | 0.03 | 2.56 | 3.51 | 4.04 | 0.04 | 3.68 | 3.75 | 3.59 |
| GRB10 | NM_001001555 | 0.46 | 1.95 | 4.32 | 4.06 | 0.32 | 3.53 | 4.72 | 3.75 |
| PRKCQ | NM_006257 | 0.18 | 0.82 | 2.37 | 0.53 | −0.01 | 3.52 | 2.79 | 0.18 |
| WWTR1 | NM_015472 | 1.22 | 1.33 | 4.00 | 3.52 | 1.54 | [symbol] | 4.50 | 3.54 |
| HOXA2 | NM_006735 | 1.03 | 2.34 | 4.63 | 4.18 | 0.93 | [symbol] | 4.33 | 3.79 |
| STOX2 | NM_020225 | −0.49 | 1.42 | 4.10 | 4.66 | −1.20 | 3.40 | 4.90 | 4.22 |
| HIST1H2AG | NM_021064 | −0.17 | 2.30 | 3.23 | 3.72 | −0.01 | 3.31 | 3.59 | 3.28 |
| HIST1H2AD | NM_021065 | 1.07 | 2.26 | 2.65 | 2.49 | 0.78 | 3.28 | 3.20 | 2.25 |
| EFNB2 | NM_004093 | −3.47 | 1.28 | 3.97 | 4.08 | −3.11 | 3.28 | 4.52 | 3.30 |
| SLC25A4 | NM_001151 | −0.35 | 1.54 | 3.46 | 3.26 | −0.30 | 3.26 | 3.89 | 2.92 |
| C1QC | NM_172369 | −0.02 | 0.81 | 1.82 | 1.38 | −0.07 | 3.26 | 4.51 | 0.65 |
| NPTXR | NM_014293 | −0.68 | 0.07 | 1.32 | 0.19 | −0.30 | 3.22 | 2.34 | −0.06 |
| LONRF3 | NM_024778 | 0.17 | 1.53 | 4.02 | 4.10 | 0.03 | 3.15 | 4.64 | 2.95 |
| ADCY5 | NM_183357 | −0.06 | −0.02 | 3.27 | 2.67 | 0.19 | 3.14 | 3.89 | 2.39 |
| HIC1 | BY798288 | 0.68 | 1.59 | 4.37 | 4.36 | 0.84 | 3.12 | 4.44 | 3.78 |
| TRIM26 | NM_003449 | −0.56 | 1.68 | 2.91 | 3.77 | −0.21 | 3.05 | 2.98 | 2.88 |
| SPOCK2 | NM_014767 | 0.46 | −0.20 | 2.75 | 2.52 | 0.54 | 3.01 | 3.88 | 1.66 |
| TMEM48 | NM_018087 | 0.95 | 1.97 | 2.43 | 2.09 | 0.76 | 3.01 | 2.97 | 1.44 |
| CHCHD10 | NM_213720 | 0.50 | 0.83 | 2.59 | 3.42 | 0.49 | 2.99 | 3.16 | 4.14 |
| ALAD | NM_001003945 | 0.06 | 1.19 | 3.01 | 3.50 | −0.13 | 2.96 | 3.45 | 2.66 |
| KDR | NM_002253 | −0.47 | 0.14 | 3.03 | 1.41 | −0.28 | 2.95 | 3.59 | 1.56 |
| LSM11 | NM_173491 | 0.90 | 1.87 | 2.42 | 2.79 | 1.20 | 2.94 | 3.07 | 1.96 |
| ATP1B1 | NM_001677 | −1.25 | 0.34 | 2.93 | 3.16 | −0.49 | 2.90 | 3.38 | 3.67 |
| NPHP1 | NM_000272 | 1.17 | 1.86 | 3.19 | 3.40 | 0.79 | 2.89 | 3.79 | 2.78 |
| CALB2 | NM_001740 | −0.18 | 0.24 | 5.21 | 6.43 | −0.19 | 2.88 | 6.17 | 7.07 |
| HIST2H2AC | NM_003517 | 0.65 | 1.80 | 2.36 | 2.13 | 0.48 | 2.88 | 2.83 | 2.00 |
| TCF7L1 | NM_031283 | −0.23 | 0.96 | 3.36 | 4.08 | −0.05 | 2.88 | 3.81 | 3.04 |
| BBS5 | NM_152384 | 0.11 | 1.78 | 3.67 | 3.25 | 0.39 | 2.86 | 4.27 | 2.26 |
| KCNC3 | NM_004977 | −0.02 | 1.61 | 3.02 | 2.80 | 0.42 | 2.84 | 3.49 | 2.15 |
| NFIX | NM_002501 | −0.27 | 1.45 | 3.86 | 3.33 | −0.25 | 2.80 | 4.30 | 3.18 |
| ZNF213 | NM_004220 | 0.39 | 1.64 | 4.79 | 2.45 | 0.28 | 2.77 | 5.03 | 2.11 |
| PLK1 | NM_005030 | 1.46 | 1.26 | 2.06 | 1.01 | 1.18 | 2.76 | 2.54 | 1.62 |
| GSTM4 | NM_147148 | 0.19 | 1.22 | 3.83 | 3.59 | −0.25 | 2.75 | 4.16 | 2.90 |
| SLC4A4 | NM_003759 | −0.65 | −0.50 | 2.62 | 2.39 | −0.64 | 2.72 | 3.05 | 1.29 |
| TMEM33 | BU567141 | −0.83 | 0.70 | 5.78 | 4.27 | −0.65 | 2.72 | 5.82 | 2.25 |
| FKBP1A | NM_054014 | −0.27 | 1.56 | 3.17 | 3.07 | −0.08 | 2.68 | 3.55 | 2.40 |
| INMT | NM_006774 | 0.42 | 0.45 | 2.61 | 2.54 | 0.14 | 2.68 | 3.27 | 1.12 |
| CELF1 | NM_198700 | 0.11 | 1.30 | 2.09 | 2.83 | 0.62 | 2.66 | 2.97 | 2.47 |
| EIF4B | NM_001417 | 0.23 | 0.77 | 5.25 | 3.16 | 0.11 | 2.65 | 5.52 | 1.24 |
| ZNF423 | NM_015069 | −0.64 | −0.21 | 4.17 | 4.49 | −0.71 | 2.63 | 4.74 | 3.79 |
| TBRG1 | NM_032811 | 0.27 | 1.17 | 2.54 | 2.30 | 0.42 | 2.62 | 3.53 | 1.88 |
| AGAP1 | NM_014914 | 0.58 | 1.37 | 2.95 | 3.66 | 0.22 | 2.56 | 3.66 | 2.84 |
| ATP2A2 | NM_001681 | −0.24 | 1.14 | 2.26 | 2.07 | 0.27 | 2.55 | 2.99 | 1.48 |
| GPR27 | THC2522889 | −0.18 | 1.34 | 2.96 | 1.76 | 0.06 | 2.54 | 2.69 | 1.10 |
| GRRP1 | NM_024869 | −0.09 | 0.71 | 2.81 | 3.24 | −0.04 | 2.53 | 3.36 | 2.57 |
| SUV39H1 | NM_003173 | −0.38 | 1.23 | 2.46 | 1.78 | −0.56 | 2.49 | 2.85 | 1.76 |
| TEX261 | NM_144582 | −0.82 | 1.46 | 2.99 | 2.59 | −0.76 | 2.48 | 3.39 | 1.93 |
| ARL6 | NM_032146 | 0.74 | 0.91 | 3.42 | 2.12 | 0.58 | 2.40 | 3.78 | 1.14 |
| SLC35A2 | NM_005660 | −0.42 | 1.01 | 3.19 | 3.57 | −0.43 | 2.38 | 3.62 | 3.11 |
| LIX1L | NM_153713 | 0.66 | 1.02 | 3.11 | 3.51 | 0.72 | 2.37 | 3.74 | 2.94 |
| TTC23L | NM_144725 | 0.21 | −0.03 | 0.56 | 1.78 | −0.08 | 2.36 | 0.67 | 1.10 |
| HISTIH2BJ | NM_021058 | 0.51 | 1.24 | 4.14 | 4.07 | 0.04 | 2.29 | 3.80 | 3.19 |
| (includes | | | | | | | | | |
| EG: 8970) | | | | | | | | | |
| LRRN2 | NM_201630 | −0.62 | 0.80 | 3.52 | 3.62 | −0.68 | 2.23 | 3.52 | 2.07 |
| DLC1 | NM_024767 | 0.74 | 1.06 | 2.65 | 3.31 | 0.66 | 2.22 | 3.51 | 3.02 |
| HS6ST2 | NM_147175 | −0.11 | 0.03 | 3.35 | 2.30 | −0.02 | 2.22 | 4.86 | 1.99 |
| THRA | NM_003250 | 0.20 | 1.05 | 3.30 | 3.80 | 0.10 | [symbol] | 3.33 | 3.06 |
| TNKS1BP1 | NM_033396 | −0.03 | 1.10 | 2.43 | 3.27 | −0.10 | 2.20 | 2.87 | 2.28 |
| C14ORF73 | NM_001077594 | 0.45 | 0.21 | 1.56 | 1.39 | 0.32 | 2.16 | 2.50 | 0.57 |
| NOTCH4 | NM_004557 | −0.39 | 0.89 | 2.32 | 1.58 | −0.30 | 2.13 | 2.60 | 1.76 |
| HIST1H2AM | NM_003514 | 0.17 | 1.07 | 1.77 | 1.92 | 0.21 | 2.10 | 2.18 | 1.49 |
| ZKSCAN4 | AK056698 | −0.20 | 0.89 | 2.74 | 2.28 | −0.05 | 2.10 | 3.22 | 1.65 |
| GJC1 | NM_005497 | −1.98 | 0.07 | 2.47 | 2.56 | −1.60 | 2.07 | 3.04 | 1.97 |
| PLXNA4 | AB046770 | −2.00 | 0.23 | 2.56 | 2.19 | −1.55 | 2.07 | 3.39 | 3.49 |
| LIPI | NM_198996 | 0.01 | 0.99 | 2.60 | 2.28 | −0.11 | 2.07 | 3.01 | 1.73 |
| (includes | | | | | | | | | |
| EG: 149998) | | | | | | | | | |
| TGM1 | NM_000359 | −0.87 | 0.89 | 1.43 | 1.86 | −1.15 | 2.05 | 1.87 | 1.41 |
| SH3PXD2A | NM_014631 | 0.66 | 0.88 | 2.63 | 3.31 | 0.65 | 2.04 | 2.92 | 3.94 |
| TMEM145 | NM_173633 | 0.75 | 0.83 | 1.90 | 1.81 | 0.52 | 2.03 | 2.51 | 1.44 |
| B3GNT6 | NM_138706 | 0.03 | 0.66 | 2.42 | 2.20 | −0.19 | 2.01 | 2.92 | 1.18 |
| EEF1D | NM_032378 | 0.16 | 0.91 | 3.37 | 3.12 | 0.07 | 2.01 | 3.43 | 2.26 |
| CDCA3 | NM_031299 | 0.45 | 0.72 | 0.92 | 0.76 | 0.24 | 2.00 | 1.85 | 1.07 |
| RPL3L | NM_005061 | −0.30 | 0.38 | 2.54 | 1.40 | −0.04 | 1.99 | 3.28 | 0.87 |
| PLK4 | NM_014264 | −0.06 | 0.58 | 1.08 | 0.15 | 0.07 | 1.89 | 2.03 | 0.09 |
| SFRS6 | NM_006275 | −0.36 | 0.49 | 2.11 | 1.58 | −0.81 | 1.87 | 2.77 | 1.51 |
| WAS | NM_000377 | −0.18 | 0.74 | 2.18 | 1.89 | −0.15 | 1.86 | 2.46 | 1.19 |
| LOC440419 | BC037244 | 0.47 | 0.78 | 1.78 | 1.42 | 0.33 | 1.86 | 2.55 | 0.62 |
| KCNG4 | NM_172347 | −0.37 | 0.30 | 4.01 | 2.55 | −0.61 | 1.84 | 4.52 | 1.84 |
| TNIK | BE893137 | −0.73 | −0.49 | 2.93 | 2.57 | −0.10 | 1.83 | 3.80 | 2.10 |
| ASPG | NM_001080464 | −0.26 | −0.13 | 2.35 | 2.15 | −0.23 | 1.79 | 2.04 | 1.12 |
| FAM167B | NM_032648 | −0.19 | 0.08 | 1.89 | 2.70 | −0.30 | 1.75 | 2.42 | 2.02 |
| EMX1 | NM_004097 | −0.62 | −0.66 | 2.54 | 1.31 | −0.79 | 1.71 | 3.95 | 1.43 |
| GALNTL1 | NM_020692 | 0.29 | −0.10 | 2.24 | 2.31 | 0.29 | 1.70 | 2.92 | 1.22 |
| C22ORF45 | BC045098 | −0.16 | 0.22 | 2.04 | 2.01 | −0.28 | 1.69 | 2.60 | 1.10 |
| LY6G6F | NM_001003693 | −0.28 | 0.04 | 2.02 | 0.49 | 0.14 | 1.68 | 2.03 | 0.14 |
| CD3E | NM_000733 | −0.52 | 0.02 | 2.86 | 2.20 | −0.61 | 1.67 | 3.77 | 1.20 |
| VAV3 | NM_006113 | 0.00 | 0.06 | 0.52 | 1.43 | −0.03 | 1.65 | 0.37 | 0.77 |
| DAP | NM_004394 | −0.31 | 0.61 | 2.97 | 2.87 | −0.26 | 1.64 | 3.30 | 2.75 |
| B4GALT2 | BC002431 | 0.15 | 0.60 | 2.21 | 1.94 | 0.17 | 1.61 | 2.56 | 1.51 |
| DFFB | NM_001004285 | 0.00 | 0.00 | 1.67 | 2.44 | −0.26 | 1.61 | 2.38 | 2.56 |
| LTC4S | NM_145867 | −0.39 | −0.03 | 1.50 | 1.80 | −0.32 | 1.57 | 1.58 | 2.29 |
| HIPK2 | BC041926 | −0.47 | 0.36 | 2.53 | 2.77 | −0.67 | 1.55 | 2.57 | 1.38 |
| SPDYE3 | NM_001004351 | −0.29 | 0.32 | 2.16 | 2.12 | 0.05 | 1.53 | 2.60 | 1.09 |
| NAA16 | NM_018527 | −0.39 | −0.16 | 0.89 | 1.00 | −0.13 | 1.51 | 0.52 | 0.73 |
| RAD54L | NM_003579 | −0.37 | 0.37 | 0.70 | 0.76 | −0.15 | 1.50 | 1.18 | 1.26 |
| ZSCAN2 | NM_181877 | 0.20 | 0.16 | 2.37 | 1.76 | 0.23 | 1.49 | 2.76 | 0.96 |
| KCTD17 | NM_024681 | −0.41 | 0.17 | 2.10 | 2.38 | −0.55 | 1.41 | 2.67 | 2.07 |
| PROKR1 | NM_138964 | −0.58 | 0.20 | 1.58 | 1.86 | −0.39 | 1.40 | 2.01 | 1.02 |
| SLC9A3R2 | NM_004785 | −0.63 | 0.07 | 2.05 | 2.97 | −0.55 | 1.40 | 2.35 | 2.55 |
| ASAH2 | NM_019893 | −0.62 | 0.21 | 2.05 | 2.47 | −0.52 | 1.36 | 2.22 | 1.50 |
| ASRGL1 | NM_025080 | −0.61 | −0.55 | 0.90 | 0.09 | −0.11 | 1.32 | 0.61 | 0.09 |
| HMGB3 | NM_005342 | −0.55 | 0.05 | 1.05 | 1.22 | −0.30 | 1.31 | 1.80 | 2.21 |
| RASEF | NM_152573 | 0.01 | 0.14 | 2.74 | 2.62 | −0.04 | 1.28 | 2.83 | 1.69 |
| UHRF1 | NM_013282 | −2.00 | −0.07 | 0.74 | −0.17 | −2.38 | 1.16 | 1.09 | −0.07 |
| PCBP4 | NM_033010 | −1.03 | 0.12 | 1.30 | 2.12 | −0.94 | 1.14 | 1.65 | 1.86 |
| BTC | NM_001729 | −0.01 | 0.07 | 0.00 | 0.05 | −0.01 | 1.12 | 0.00 | −0.01 |
| JPH2 | NM_020433 | −0.57 | −1.50 | 1.20 | 0.45 | −0.62 | 1.10 | 1.94 | 0.05 |
| CCR3 | NM_001837 | −0.35 | −0.17 | 0.71 | 0.75 | −0.42 | 1.09 | 1.26 | 0.07 |
| CCNA2 | NM_001237 | −0.06 | −0.27 | 0.08 | −2.05 | −0.08 | 0.95 | 0.81 | −0.77 |
| DUSP10 | NM_007207 | −0.08 | −0.59 | 0.58 | 0.01 | −0.20 | 0.89 | 1.12 | 0.39 |
| PTPLA | NM_014241 | −1.39 | −0.35 | 1.25 | 1.46 | −1.34 | 0.89 | 2.00 | 0.82 |
| MGC16703 | NM_145042 | −1.17 | −0.51 | 0.11 | −1.32 | −1.30 | 0.86 | 0.94 | −1.30 |
| CUEDC1 | NM_017949 | −0.61 | −0.20 | 1.12 | 0.96 | −0.75 | 0.82 | 1.56 | 0.29 |
| PPYR1 | NM_005972 | −1.07 | −0.59 | 0.85 | 0.32 | −0.68 | 0.46 | 1.12 | −0.15 |
| SPC25 | NM_020675 | −0.67 | −0.56 | 0.18 | −0.95 | −0.76 | 0.45 | 0.70 | −0.26 |
| FGF18 | NM_003862 | −1.55 | −1.96 | −0.24 | 0.63 | −2.01 | [symbol] | 0.55 | 0.20 |

Down regulated and differentially expressed at 2 days

| | | | | | | | | | |
|---|---|---|---|---|---|---|---|---|---|
| GPR1 | NM_005279 | −1.24 | −3.02 | −3.37 | −3.81 | −1.76 | −4.85 | −5.21 | −5.18 |
| ASPN | NM_017680 | 0.29 | −2.13 | 2.52 | 4.13 | 0.34 | −4.27 | 2.18 | 4.65 |
| CBWD6 | AF293368 | 0.15 | −2.34 | −4.24 | −4.75 | 0.06 | −3.77 | −4.62 | −4.71 |
| ZNF706 | NM_016096 | −0.49 | −0.72 | −0.99 | −0.34 | −0.71 | −2.92 | −1.97 | −1.06 |
| NCOR1 | NM_006311 | −0.82 | −1.50 | −2.66 | −3.06 | −0.71 | −2.92 | −3.49 | −2.79 |
| KIAA1841 | BC039298 | −1.83 | −1.66 | −3.06 | −1.74 | −1.66 | −2.88 | −3.18 | −0.92 |
| SMARCA4 | NM_003072 | −0.92 | −1.40 | −1.67 | −3.54 | −1.06 | −2.69 | −1.69 | −1.84 |
| USP25 | NM_013396 | −0.83 | −0.58 | −0.99 | −2.22 | −0.72 | −2.65 | −1.15 | −2.80 |
| DYM | NM_017653 | 0.52 | −1.11 | −0.91 | −0.85 | 0.29 | −2.64 | −1.44 | −0.90 |
| MPPE1 | NM_023075 | −0.80 | −0.76 | −1.56 | −1.62 | −0.84 | −2.63 | −3.19 | −1.39 |
| PROS1 | NM_000313 | −0.15 | −0.65 | −0.98 | −2.47 | −0.17 | −2.51 | −3.19 | −3.24 |
| MSTO1 | NM_018116 | 0.25 | −1.08 | −1.63 | −1.24 | −0.11 | −2.50 | −1.27 | −0.08 |
| AKT2 | NM_001626 | −0.46 | −0.56 | −1.81 | −1.35 | −0.61 | −2.46 | −0.33 | −1.07 |
| OGN | NM_033014 | 0.52 | −0.60 | 1.00 | 3.19 | 0.36 | −2.23 | 0.38 | 3.18 |
| TTL | NM_153712 | −0.64 | −0.78 | −2.46 | −2.02 | −0.48 | −2.20 | −2.50 | −1.06 |
| MBNL2 | NM_144778 | 0.13 | −0.30 | −1.76 | −2.49 | 0.16 | −2.20 | −2.00 | −2.04 |
| SVEP1 | BC030816 | 0.39 | 0.30 | −2.79 | 0.25 | 0.58 | −1.99 | −3.28 | −1.03 |
| ZDHHC20 | NM_153251 | 0.02 | −0.31 | −2.46 | −1.67 | −0.06 | −1.97 | −3.14 | −0.98 |
| CDKAL1 | ENST00000378610 | 0.13 | −0.11 | −0.41 | −0.23 | −0.11 | −1.86 | −0.22 | −0.18 |
| CACNB4 | NM_001005747 | 0.35 | −0.28 | −0.40 | −0.60 | 0.38 | −1.74 | −2.41 | −2.05 |
| PRKACB | NM_002731 | −0.39 | −0.53 | −1.02 | −1.86 | −0.39 | −1.58 | −1.61 | −1.54 |
| ASAH1 | NM_004315 | −0.13 | −0.42 | −0.82 | −1.63 | −0.22 | −1.50 | −1.40 | −2.40 |
| KIAA1715 | NM_030650 | 0.13 | −0.30 | 0.17 | −2.19 | −0.10 | −1.34 | −0.43 | −2.56 |
| MIA3 | ENST00000320831 | 0.66 | 0.07 | −1.79 | −1.52 | 0.49 | −1.31 | −2.22 | −1.09 |
| ECM2 | NM_001393 | 0.55 | 0.45 | 1.61 | 2.50 | 0.52 | −1.10 | 0.76 | 1.39 |
| FAM134B | NM_019000 | 1.84 | 1.39 | −1.47 | −0.56 | 1.57 | −0.27 | −1.40 | −0.60 |
| AGTR1 | NM_031850 | 2.56 | 2.21 | −0.11 | 1.02 | 2.44 | 0.01 | −0.42 | −0.42 |

Upregulated and differentially expressed at 8 days

| | | | | | | | | | |
|---|---|---|---|---|---|---|---|---|---|
| GPSM3 | NM_022107 | −0.10 | 4.10 | 5.42 | 4.89 | −0.04 | 5.07 | 7.07 | 3.98 |
| HBD | NM_000519 | −0.22 | 1.27 | 2.97 | 5.40 | −0.54 | 3.47 | 6.28 | 1.82 |
| CXCR3 | NM_001504 | −0.33 | 3.58 | 4.07 | 5.42 | 0.15 | 3.99 | 6.19 | 4.81 |
| PPP3R1 | NM_000945 | 0.07 | 3.33 | 4.50 | 4.31 | 0.35 | 4.42 | 5.85 | 3.80 |
| FAM5C | NM_199051 | −0.59 | −0.59 | 1.17 | 2.78 | −0.60 | 0.94 | 5.48 | 5.04 |
| ITGB1BP3 | NM_014446 | −0.08 | 1.43 | 2.21 | 5.37 | −0.11 | 2.47 | 5.13 | 4.52 |
| ANO1 | NM_018043 | −0.04 | 0.97 | 1.40 | 4.46 | 0.09 | 0.33 | 4.72 | 8.09 |
| CEL | NM_001807 | −0.56 | 1.43 | 2.45 | 4.37 | −0.27 | 1.93 | 4.62 | 3.57 |
| GPR20 | NM_005293 | 0.02 | 1.86 | 2.59 | 2.28 | 0.15 | 2.48 | 4.24 | 1.64 |
| IRX1 | NM_024337 | −0.57 | 0.70 | 2.08 | 3.62 | −0.04 | 1.60 | 3.86 | 2.85 |
| GATA3 | NM_001002295 | 0.04 | 0.48 | 0.30 | 6.23 | −0.07 | 1.48 | 3.70 | 4.58 |
| RASIP1 | NM_017805 | −0.32 | 1.00 | 1.69 | 3.52 | −0.15 | 1.68 | 3.68 | 2.60 |
| P2RY4 | NM_002565 | −0.18 | 0.76 | 1.85 | 3.59 | −0.18 | 1.56 | 3.62 | 2.77 |
| NKD2 | NM_033120 | 1.32 | −0.46 | 0.64 | 5.53 | 1.35 | 0.03 | 3.57 | 9.39 |
| ERC2 | NM_015576 | −0.02 | 0.66 | 0.36 | 0.26 | −0.04 | 0.48 | 3.55 | 0.81 |
| GJB2 | NM_004004 | 1.83 | 0.48 | 1.34 | 2.35 | 1.80 | 0.43 | 3.52 | 5.44 |
| HOXB8 | NM_024016 | −0.03 | 0.72 | 0.65 | 0.33 | −0.04 | 0.18 | [symbol] | 0.38 |
| CNIH2 | NM_182553 | −0.25 | 0.68 | 1.62 | 2.85 | −0.17 | 1.18 | 3.31 | 2.16 |
| EGLN3 | NM_022073 | 4.82 | 1.12 | 0.36 | 3.16 | 3.61 | 1.10 | 3.28 | 5.23 |
| SLN | NM_003063 | 0.03 | 0.13 | 0.06 | 0.54 | 0.02 | 0.11 | 3.18 | 7.86 |
| RHO | NM_000539 | −0.18 | 2.04 | 0.35 | 1.26 | −0.22 | 1.01 | 3.10 | 1.29 |
| GPR64 | NM_005756 | −0.42 | 0.19 | 1.83 | 0.44 | −0.31 | 1.37 | 3.09 | 1.43 |
| M96686 | M96686 | −0.57 | 0.21 | −0.02 | 0.76 | −0.15 | 0.46 | 2.99 | 2.19 |
| MGC50722 | NM_203348 | −0.28 | −0.28 | 0.24 | 2.52 | −0.29 | 0.63 | 2.69 | 1.44 |
| FRK | NM_002031 | −0.36 | 0.03 | 0.75 | 0.28 | −0.31 | 0.32 | 2.64 | −0.01 |
| NPM2 | NM_182795 | 0.04 | −0.26 | 0.77 | 1.90 | −0.02 | 0.56 | 2.56 | 1.10 |
| ENPP1 | NM_006208 | −0.75 | −0.40 | 0.81 | 2.12 | −0.65 | −0.90 | 2.45 | 6.01 |
| SLAMF7 | NM_021181 | −0.11 | −0.10 | 0.83 | 1.61 | 0.00 | 0.36 | 2.42 | 0.99 |
| LOC100129572 | AK096041 | 0.00 | 0.00 | 0.05 | 0.11 | −0.01 | 0.01 | 2.38 | 0.46 |
| RP11-165I9.3 | ENST00000381857 | −0.26 | 0.28 | 0.24 | −0.40 | −0.34 | −0.18 | 2.37 | −0.49 |
| CHRM3 | NM_000740 | 0:34 | 0.23 | 0.22 | −0.13 | 0.41 | −0.07 | 2.37 | −0.13 |
| DBX2 | NM_001004329 | 0.00 | 0.06 | 0.53 | 0.35 | 0.00 | 1.01 | 2.36 | 0.06 |
| ODF3 | NM_053280 | 0.10 | 0.01 | 0.30 | 0.69 | 0.15 | 0.15 | 2.36 | 0.48 |
| PI15 | NM_015886 | −1.08 | −0.76 | −0.11 | 1.69 | −0.26 | −0.34 | 2.32 | 0.99 |
| MAGEA5 | NM_021049 | −0.56 | −0.81 | 0.21 | 1.53 | 0.00 | −0.14 | 2.30 | 0.73 |
| SLC41A1 | NM_173854 | 0.28 | 0.65 | 0.25 | −0.04 | 0.84 | 0.06 | 2.28 | 0.07 |
| ACTRT2 | NM_080431 | −0.41 | −0.69 | 0.34 | 2.41 | 0.13 | 0.43 | 2.18 | 1.45 |
| IPCEF1 | NM_015553 | 0.00 | 0.07 | 0.10 | −0.02 | −0.03 | 0.06 | 2.17 | −0.04 |
| RSPH10B | NM_173565 | −0.03 | 0.00 | −0.01 | 0.14 | −0.03 | 0.11 | 2.17 | 0.30 |
| CNTN2 | NM_005076 | −0.04 | 0.13 | 0.00 | 0.17 | −0.06 | 0.05 | 2.16 | −0.09 |
| LOC100290146 | ENST00000390622 | 0.02 | 0.08 | 0.20 | 0.19 | −0.01 | 0.07 | 2.06 | 0.11 |
| CCR4 | NM_005508 | −0.10 | −0.18 | −0.08 | −0.15 | −0.17 | −0.13 | 1.90 | −0.04 |
| DQX1 | NM_133637 | 0.01 | 0.04 | 0.12 | 0.31 | −0.01 | 0.05 | 1.85 | 0.20 |
| HDAC9 | NM_058177 | 0.00 | 0.02 | 0.05 | 0.07 | −0.01 | −0.03 | 1.83 | 0.00 |
| HECW1 | NM_015052 | 0.01 | 0.03 | 0.07 | 0.15 | 0.00 | −0.02 | 1.75 | 0.34 |
| DLG2 | NM_001364 | 0.02 | 0.13 | 0.24 | 0.40 | 0.01 | 0.49 | 1.73 | 0.49 |
| SLC6A13 | NM_016615 | −0.37 | −0.79 | −0.53 | 0.10 | −0.50 | −0.73 | 1.34 | −0.19 |
| HIST1H3B | NM_003537 | 0.12 | −0.21 | −0.52 | −0.12 | −0.23 | 0.14 | 1.25 | 0.11 |
| CREBBP | NM_004380 | 0.07 | 0.46 | 0.05 | 0.06 | 0.08 | 0.17 | 1.18 | −0.02 |
| IL3RA | NM_002183 | 0.26 | −1.23 | −0.41 | 0.04 | −0.03 | −0.54 | 1.16 | −0.49 |
| APP | NM_000484 | 0.23 | −0.60 | −0.48 | −0.90 | 0.14 | −0.45 | 0.55 | −0.18 |
| MKS1 | NM_017777 | −0.20 | −0.84 | −0.72 | 0.39 | −0.33 | −0.87 | 0.43 | 0.24 |
| KRT33B | NM_002279 | −2.10 | −2.20 | −1.97 | −1.92 | −1.77 | −2.40 | −0.28 | −2.49 |
| ZC3H7B | NM_017590 | −1.48 | −2.03 | −1.53 | −2.05 | −1.40 | −1.51 | −0.29 | −1.51 |

Down regulated and differentially expressed at 8 days

| | | | | | | | | | |
|---|---|---|---|---|---|---|---|---|---|
| CRIM1 | NM_016441 | −1.42 | −2.71 | −3.34 | −4.83 | −0.99 | −3.15 | −5.41 | −5.91 |
| AKAP12 | NM_144497 | 0.66 | −2.69 | −2.87 | −2.60 | 1.01 | −2.27 | −4.99 | −4.06 |
| CST6 | NM_001323 | 0.01 | −1.12 | −2.49 | −5.19 | 0.18 | −1.45 | −4.77 | −5.43 |
| LOC730101 | AK095359 | −0.40 | −1.30 | −2.10 | −3.28 | −0.80 | −2.11 | −4.73 | −3.39 |
| FEM1C | NM_020177 | 1.51 | −1.27 | −2.00 | −2.94 | 1.53 | −1.31 | −4.49 | −2.78 |
| K1TLG | NM_000899 | −1.34 | −1.42 | −1.38 | −3.73 | −1.43 | −2.17 | −4.48 | −4.39 |
| LACTB | NM_171846 | −0.34 | −1.35 | −2.58 | −2.12 | −0.20 | −1.71 | −4.42 | −2.15 |
| GYG1 | NM_004130 | −0.87 | −0.85 | −1.86 | −2.27 | −0.76 | −1.63 | −4.36 | −2.34 |
| AKIRIN1 | NM_024595 | −2.03 | −1.33 | −2.35 | −1.59 | −1.48 | −1.83 | −4.31 | −1.31 |
| DYNC1LI1 | NM_016141 | −0.90 | −1.60 | −2.40 | −2.95 | −0.92 | −1.96 | −4.29 | −2.62 |
| CPNE8 | NM_153634 | 0.18 | −1.14 | −1.90 | −2.48 | 0.22 | −1.47 | −4.08 | −2.31 |
| LOC441016 | ENST00000312008 | −1.12 | −1.92 | −2.38 | −0.92 | −0.91 | −2.28 | −4.07 | −0.63 |
| CYP2U1 | NM_183075 | −0.74 | −1.69 | −1.71 | −1.76 | −0.84 | −1.67 | −3.97 | −2.17 |
| VPS41 | BX648347 | −0.52 | −1.19 | −1.58 | −0.18 | −0.33 | −1.59 | −3.97 | 0.30 |
| ERCC6 | NM_000124 | −1.49 | −1.99 | −2.82 | −3.43 | −1.64 | −2.61 | −3.96 | −3.61 |
| SURF6 | NM_006753 | −1.79 | −1.44 | −1.73 | −1.14 | −1.79 | −1.63 | −3.96 | −1.04 |
| MREG | NM_018000 | −0.12 | −0.76 | −1.41 | −1.93 | −0.27 | −1.24 | −3.90 | −2.69 |
| MAPKAP1 | NM_001006617 | −1.19 | −1.11 | −1.55 | −2.34 | −1.33 | −1.82 | −3.86 | −1.36 |
| C21ORF91 | NM_017447 | −0.95 | −1.59 | −1.91 | −2.32 | −1.42 | −2.16 | −3.78 | −2.27 |
| MAP9 | NM_001039580 | −0.58 | −0.94 | −2.35 | −1.93 | −0.73 | −1.02 | −3.73 | −1.18 |
| TMEM192 | NM_152681 | 0.37 | −0.60 | −1.49 | −1.35 | 0.10 | −0.98 | −3.67 | −1.23 |
| TMEM17 | NM_198276 | −1.11 | −0.66 | −1.99 | −1.25 | −1.00 | −1.23 | −3.67 | −1.47 |
| RMND1 | NM_017909 | 0.96 | −0.72 | −1.59 | −1.77 | 1.23 | −1.09 | −3.64 | −1.98 |
| TAF9B | NM_015975 | −2.11 | −1.58 | −1.66 | −1.15 | −1.77 | −1.48 | −3.64 | −1.08 |
| ORC5L | NM_181747 | −1.10 | −1.47 | −2.04 | −2.43 | −1.15 | −1.98 | −3.55 | −2.07 |
| PPFIA1 | NM_003626 | 0.21 | −0.71 | −1.42 | −1.04 | 0.34 | −1.52 | −3.54 | −0.46 |
| M6PR | NM_002355 | −0.50 | −1.51 | −2.40 | −1.97 | −0.31 | −1.66 | −3.53 | −1.93 |
| ASB8 | NM_024095 | −0.55 | −0.90 | −1.37 | −0.50 | −0.74 | −1.38 | −3.53 | −0.76 |
| SAMD9 | NM_017654 | 1.38 | −0.32 | −1.28 | −2.36 | 1.03 | −0.36 | −3.47 | −2.90 |
| LOC100130506 | AF075027 | −0.88 | −1.45 | −2.31 | −1.63 | −0.45 | −1.49 | −3.45 | −2.95 |
| PSTK | NM_153336 | −0.06 | −1.05 | −1.88 | −1.94 | −0.25 | −1.63 | −3.38 | −1.60 |
| MPHOSPH8 | NM_017520 | 0.25 | −0.44 | −0.08 | 0.90 | −0.54 | −1.19 | −3.37 | 1.00 |
| GDAP2 | NM_017686 | −0.98 | −1.18 | −1.50 | −0.83 | −0.47 | −1.60 | −3.37 | −0.74 |
| HSD17B7 | NM_016371 | 0.84 | −0.86 | −1.70 | −2.26 | 0.69 | −1.69 | −3.34 | −1.61 |
| LOC392288 | XM_373277 | −0.04 | −1.37 | −1.88 | −3.46 | 0.16 | −1.46 | −3.31 | −2.87 |
| IKZF5 | NM_022466 | 0.13 | −0.72 | −0.79 | −1.31 | 0.28 | −0.84 | −3.25 | −1.28 |
| TP53INP2 | NM_021202 | 0.12 | −0.22 | −1.15 | −1.32 | 0.50 | −0.47 | −3.25 | −0.36 |
| LOC283788 | AK127309 | −0.45 | −0.25 | −1.36 | −0.92 | −0.89 | −0.95 | −3.21 | −0.23 |
| C7ORF64 | NM_032120 | 0.72 | −0.69 | −1.25 | −1.43 | 0.61 | −0.67 | −3.19 | −1.53 |
| GFRA1 | NM_005264 | 0.14 | −0.43 | −2.07 | −1.83 | 0.11 | −1.23 | −3.18 | −4.31 |
| RBM38 | NM_017495 | −0.86 | −0.89 | −0.86 | −1.41 | −0.82 | −0.96 | −3.18 | −0.62 |
| TXNL4B | NM_017853 | 0.84 | −0.69 | −1.52 | −0.53 | 0.61 | −0.71 | −3.15 | 0.15 |
| ARHGAP29 | NM_004815 | −0.85 | −1.33 | −1.86 | −2.30 | −0.91 | −1.51 | −3.09 | −3.72 |
| CLCN3 | NM_001829 | −0.01 | −0.27 | −1.14 | −1.52 | 0.32 | −0.99 | −3.08 | −1.30 |
| UVRAG | NM_003369 | 0.07 | −0.77 | −0.75 | −1.35 | 0.20 | −1.38 | −3.06 | −1.12 |
| EIF2S1 | NM_004094 | −1.00 | −0.79 | −1.09 | −1.05 | −0.77 | −0.80 | −3.00 | −0.81 |
| ZNF532 | NM_018181 | 0.07 | −0.45 | −0.69 | −0.44 | 0.22 | −0.75 | −2.95 | 0.02 |
| TMEM109 | NM_024092 | −0.68 | −0.93 | −1.67 | −2.55 | −0.82 | −1.74 | −2.94 | −1.62 |
| SLC8A1 | NM_021097 | −0.50 | −0.37 | −1.06 | −0.81 | −0.69 | −0.94 | −2.92 | −1.77 |
| NAA30 | NM_001011713 | −0.18 | −1.04 | −1.50 | −1.70 | 0.02 | −1.10 | −2.91 | −1.34 |
| ZNF696 | NM_030895 | −1.13 | −0.47 | −0.72 | −0.04 | −1.24 | −0.54 | −2.87 | −0.04 |
| CAPRIN2 | NM_001002259 | −0.48 | −0.56 | −1.08 | −0.94 | −0.41 | −0.96 | −2.82 | −0.42 |
| ZNF823 | NM_001080493 | −0.93 | −0.83 | −1.47 | −0.62 | −0.99 | −0.93 | −2.82 | −0.83 |
| NMNAT1 | NM_022787 | −0.09 | −0.89 | −1.43 | −0.69 | −0.12 | −1.33 | −2.77 | −0.52 |
| ZNF322B | NM_199005 | −0.43 | −0.57 | −1.07 | −1.27 | −0.37 | −1.34 | −2.74 | −1.10 |
| C1ORF55 | NM_152608 | 0.22 | −0.02 | −0.84 | −1.07 | 0.79 | 0.34 | −2.71 | −0.85 |
| ZNF33A | NM_006974 | −0.14 | −0.93 | −1.20 | −0.12 | 0.08 | −0.61 | −2.68 | −0.13 |
| TSPYL1 | NM_003309 | −0.60 | −0.26 | −1.02 | −1.92 | −0.60 | −1.02 | −2.67 | −1.88 |
| GNPDA1 | NM_005471 | 0.37 | 0.22 | −0.11 | 0.46 | 0.16 | −0.29 | −2.61 | 0.13 |
| C3ORF19 | NM_016474 | 0.40 | −0.39 | −0.69 | −0.51 | 0.15 | −0.55 | −2.61 | −0.40 |
| CEP250 | NM_007186 | 1.21 | −0.62 | −0.73 | −1.93 | 1.61 | −0.71 | −2.59 | −1.46 |
| ZNF498 | NM_145115 | −0.55 | −1.50 | −0.64 | −0.40 | −0.59 | −0.80 | −2.58 | −0.41 |
| NUDT15 | NM_018283 | −0.64 | −1.00 | −1.55 | −1.60 | −0.45 | −1.45 | −2.57 | −1.30 |
| CRBN | NM_016302 | 0.46 | −0.81 | −0.92 | −1.60 | 0.39 | −0.94 | −2.56 | −1.81 |
| TTC14 | NM_133462 | −1.05 | −0.74 | −0.90 | −0.48 | −0.89 | −0.62 | −2.55 | −0.27 |
| C4ORF49 | NM_032623 | −0.30 | −0.14 | −0.27 | −0.16 | −0.42 | −0.63 | −2.49 | −3.11 |
| APOBEC3C | NM_014508 | 0.45 | 0.08 | −0.06 | −1.49 | −0.09 | −0.62 | −2.47 | −1.74 |
| PMS2 | NM_000535 | 1.71 | 0.30 | −0.97 | −0.34 | 1.52 | 0.13 | −2.45 | 0.14 |
| ZNF227 | NM_182490 | 0.30 | −0.41 | −0.60 | −0.33 | −0.04 | −0.63 | −2.43 | −0.35 |
| RBM12B | NM_203390 | −0.85 | −0.76 | −1.08 | −0.78 | −0.60 | −0.65 | −2.42 | −0.33 |
| METTL2B | NM_018396 | −1.01 | −0.53 | −0.82 | −0.71 | −1.41 | −0.68 | −2.41 | −0.36 |
| TADA2B | ENST00000310074 | −0.27 | 0.01 | −0.52 | −0.84 | 0.12 | −0.42 | −2.40 | −0.36 |
| ZNF614 | NM_025040 | 0.96 | −0.19 | −0.81 | −0.64 | 1.35 | −0.32 | −2.38 | −0.29 |
| C11ORF57 | NM_018195 | 0.18 | −0.31 | −0.68 | −0.30 | 0.04 | −0.25 | −2.37 | −0.26 |
| CDKN2AIP | NM_017632 | −0.85 | −0.27 | −0.75 | −1.04 | −0.56 | −0.51 | −2.34 | −1.19 |
| ABCD1 | NM_000033 | −0.49 | −1.07 | −1.22 | −1.76 | −0.72 | −1.08 | −2.33 | −1.00 |
| TRAK2 | NM_015049 | 0.05 | −0.29 | −1.05 | −1.01 | −0.32 | −1.03 | −2.33 | −0.62 |
| ZNF433 | NM_001080411 | 1.02 | −0.79 | −1.12 | −0.25 | 1.50 | −1.10 | −2.32 | −0.06 |
| C5ORF22 | NM_018356 | −1.27 | −0.93 | −1.12 | −0.82 | −0.56 | −1.02 | −2.29 | −0.81 |
| ZNF527 | AK091585 | 0.40 | −0.49− | 0.27 | 0.18 | 0.29 | −0.78 | −2.28 | 0.37 |
| LOC100129122 | AF339771 | 0.18 | 0.75 | −0.65 | −0.24 | 0.43 | 1.00 | −2.25 | −0.41 |
| ZNF135 | NM_003436 | −0.06 | −0.44 | −0.81 | −0.22 | −0.43 | −0.33 | −2.14 | 0.13 |
| TNFRSF10B | NM_003842 | 0.95 | 0.00 | −0.35 | −0.23 | 1.51 | 0.18 | −2.13 | −0.48 |
| USP51 | NM_201286 | 0.32 | 0.20 | −0.41 | 0.26 | −0.05 | 0.14 | −2.12 | 0.28 |
| TRAPPC2L | BC011369 | 0.91 | 0.09 | −0.20 | −0.82 | 0.45 | 0.01 | −2.10 | −0.53 |
| FAM178A | NM_018121 | −0.55 | −0.31 | −0.65 | −0.34 | −1.01 | −0.34 | −2.10 | −0.06 |
| PRKAB2 | NM_005399 | 1.68 | 0.03 | −0.42 | −1.47 | 1.58 | −0.58 | −2.08 | −1.06 |
| C2ORF60 | NM_001039693 | 0.30 | 0.35 | −0.36 | 0.85 | 0.21 | 0.07 | −2.07 | 0.96 |
| FRZB | NM_001463 | 0.98 | 0.27 | −0.16 | 0.23 | 0.86 | 0.00 | [symbol] | 0.01 |
| CD59 | NM_203330 | −0.41 | −0.76 | −0.97 | −1.64 | −0.43 | −1.04 | −2.04 | −2.79 |
| ZNF232 | NM_014519 | 0.28 | −0.24 | −0.93 | −0.35 | 0.14 | −0.35 | −2.03 | −0.14 |
| DNMBP | BC041628 | 0.44 | −0.41 | −0.98 | −1.85 | 0.22 | −0.60 | −2.02 | −2.25 |
| UBXN7 | ENST00000296328 | 0.74 | 0.25 | −0.10 | −1.12 | 0.63 | −0.22 | −1.98 | −1.03 |
| ZNF630 | NM_001037735 | 0.78 | 0.31 | −0.14 | −0.45 | 0.02 | 0.21 | −1.86 | −0.14 |
| LSS | NM_001001438 | 0.89 | 0.89 | −0.11 | −0.42 | 1.01 | 0.75 | −1.86 | −1.03 |
| TMEM81 | NM_203376 | −0.46 | 1.14 | 0.34 | −0.62 | −0.07 | 0.40 | −1.77 | 0.43 |
| ERP44 | NM_015051 | 0.17 | 0.01 | 0.27 | 0.49 | 0.35 | −0.05 | −1.73 | 0.47 |
| LOC100131053 | AK095564 | −1.30 | 0.50 | −0.12 | −0.07 | −1.16 | 0.09 | −1.72 | −0.06 |
| HOXC10 | NM_017409 | −0.37 | −0.52 | −0.54 | −1.31 | −0.61 | −0.55 | [symbol] | −1.59 |
| TTC17 | BC033000 | 0.61 | −0.01 | −0.24 | −0.41 | 0.86 | −0.11 | −1.69 | −0.43 |
| FZR1 | NM_016263 | 0.59 | 0.56 | 0.01 | −0.78 | 0.19 | 0.31 | −1.65 | −0.13 |
| C2ORF49 | ENST00000258457 | 0.60 | −0.21 | −0.62 | −0.80 | 0.80 | −0.42 | −1.62 | −0.71 |
| LOC338620 | BC043009 | −0.65 | −0.38 | −0.41 | −2.69 | −0.74 | −0.41 | −1.61 | −1.66 |
| DKFZP667E0512 | AL713660 | −1.00 | 0.69 | 1.42 | −0.34 | −0.96 | 0.42 | −1.54 | 0.93 |
| LOC730183 | BM932296 | 0.07 | 1.25 | −0.19 | −0.06 | 0.22 | 1.46 | −1.53 | 0.23 |
| RBM43 | AV652851 | 2.12 | 1.30 | 0.62 | 0.57 | 1.84 | 1.12 | −1.53 | 0.03 |
| TRIM37 | NM_001005207 | −0.47 | 0.26 | 0.12 | −1.41 | −0.56 | −0.22 | −1.46 | −1.57 |
| IPP | NM_005897 | −0.33 | −0.03 | −0.21 | −0.48 | −0.67 | −0.37 | −1.39 | −0.15 |
| C20ORF106 | NM_001012971 | 0.86 | 1.23 | 0.39 | −1.01 | 1.63 | 1.43 | −1.36 | −0.51 |
| CSNK1E | NM_52221 | 0.89 | 0.29 | 0.22 | 0.91 | 1.21 | 0.33 | −1.29 | 1.53 |
| GABPB2 | ENST00000368918 | 0.76 | 0.10 | −0.16 | 0.40 | 0.43 | −0.04 | −1.22 | 0.17 |
| MARCH8 | ENST00000374390 | 1.24 | 0.54 | 0.05 | −0.22 | 0.91 | 0.27 | −1.05 | −0.62 |
| MKNK2 | NM_017572 | 1.34 | 0.25 | 0.02 | 0.27 | 1.56 | 0.40 | −1.04 | −0.04 |
| LOC153546 | AK055939 | 0.92 | 0.17 | 0.18 | 0.21 | 0.98 | 0.50 | −1.02 | 0.19 |
| TECPR2 | NM_014844 | −1.04 | 0.44 | 0.63 | 0.62 | −0.68 | 0.34 | −1.01 | 1.05 |
| C18ORF56 | NM_001012716 | 0.61 | 0.54 | 0.90 | −0.47 | 0.74 | 0.85 | −0.95 | −0.75 |
| VPS29 | BC032462 | 0.45 | 0.77 | 0.64 | 3.55 | 0.21 | 0.23 | −0.78 | 3.66 |
| SCARB2 | NM_005506 | 0.69 | 0.89 | 0.37 | −0.65 | 0.55 | 0.39 | −0.74 | −0.81 |
| C8ORF44 | NM_019607 | 0.99 | 0.35 | 0.84 | 1.04 | 1.02 | 0.60 | −0.71 | 0.62 |
| BTN2A1 | AB209777 | 0.47 | 1.10 | 1.00 | 0.38 | 0.46 | 1.18 | −0.10 | 0.86 |
| C7ORF53 | NM_182597 | 2.01 | 2.27 | 2.25 | 2.26 | 2.46 | 2.16 | −0.06 | 2.03 |
| BCL2A1 | NM_004049 | 3.35 | 0.69 | −2.21 | −3.10 | 4.83 | 2.42 | 0.19 | −0.88 |
| UNQ6228 | AY358248 | 1.02 | 4.24 | 2.92 | 4.20 | 1.54 | 3.82 | 0.66 | 4.21 |
| TRIB1 | NM_025195 | 1.40 | 1.79 | −0.30 | 0.09 | 1.14 | 1.95 | 0.73 | 3.35 |

Upregulated and differentially expressed at 18 days

| | | | | | | | | | |
|---|---|---|---|---|---|---|---|---|---|
| SPP1 | NM_000582 | −0.32 | −0.23 | −0.57 | −0.40 | −0.04 | −0.45 | 0.52 | [symbol] |
| MIAT | NR_003491 | 2.76 | 5.10 | 4.67 | 7.36 | 1.69 | 4.40 | 7.12 | 11.07 |
| DMP1 | NM_004407 | −0.02 | 0.08 | 0.01 | −0.08 | −0.02 | 0.05 | 0.08 | [symbol] |
| NKD2 | NM_033120 | 1.32 | −0.46 | 0.64 | 5.53 | 1.35 | 0.03 | 3.57 | 9.39 |
| ANO1 | NM_018043 | −0.01 | 1.25 | 2.51 | 5.08 | 0.41 | 1.23 | 4.52 | 8.89 |
| LEF1 | NM_016269 | −1.10 | −0.86 | 0.50 | 2.19 | −0.75 | 1.08 | 1.09 | 8.14 |
| RGS16 | NM_002928 | 3.51 | 0.09 | 1.52 | 3.33 | 4.20 | 1.38 | 4.20 | 7.88 |
| SLN | NM_003063 | 0.03 | 0.13 | 0.06 | 0.54 | 0.02 | 0.11 | 3.18 | 7.86 |
| TNFSF11 | NM_003701 | −0.02 | 0.17 | 0.03 | 2.30 | −0.04 | 0.11 | 1.90 | [symbol] |
| MMP11 | NM_005940 | 0.47 | 1.10 | 3.21 | 4.75 | 0.89 | 1.03 | 3.46 | 7.75 |
| AOC3 | NM_003734 | 0.68 | 1.83 | 0.62 | 3.27 | 0.40 | 0.57 | 1.40 | 7.68 |
| SSTR2 | NM_001050 | 1.63 | 0.78 | 1.52 | 3.15 | 2.05 | 0.62 | 2.06 | 7.53 |
| C1ORF187 | ENST00000294485 | −0.13 | 0.50 | 2.47 | 3.06 | −0.22 | 1.33 | 3.20 | 7.27 |
| KANK4 | NM_181712 | −0.25 | −0.20 | 0.23 | −0.21 | −0.09 | −0.04 | 0.30 | 6.94 |
| C21ORF96 | AK024509 | 0.76 | 2.80 | 1.60 | 3.36 | 0.77 | 2.83 | 3.31 | 6.69 |
| SALL4 | NM_020436 | 1.39 | 2.15 | 2.26 | 3.13 | 1.55 | 2.60 | 3.54 | 6.64 |
| MEGF10 | NM_032446 | 0.23 | −0.48 | −0.50 | −0.38 | −0.16 | −0.50 | −0.26 | 6.37 |
| BMP8A | NM_181809 | −0.04 | 0.65 | 2.12 | −0.07 | −0.14 | 0.31 | 0.72 | [symbol] |
| CRYGS | NM_017541 | 0.05 | 1.69 | 3.22 | 4.78 | 0.14 | 1.45 | 2.76 | 6.28 |
| PMEPA1 | NM_020182 | 0.42 | 2.09 | 2.50 | 4.07 | 0.80 | 2.15 | 3.42 | 6.19 |
| MDFI | NM_005586 | −0.42 | 0.87 | 1.49 | 2.68 | 0.06 | 0.29 | 2.17 | 6.18 |
| SP7 | NM_152860 | 0.08 | 0.91 | 0.42 | −0.21 | −0.04 | 0.46 | 1.04 | [symbol] |
| ENPP1 | NM_006208 | −0.75 | −0.40 | 0.81 | 2.12 | −0.65 | −0.90 | 2.45 | 6.01 |
| HOXD1 | NM_024501 | 0.40 | 0.26 | 0.01 | 1.01 | 0.44 | −0.16 | 1.29 | 5.99 |
| CHN1 | NM_001822 | 0.44 | 0.74 | 0.60 | 2.54 | 0.12 | −0.98 | 1.95 | 5.91 |
| DIO2 | NM_013989 | −0.06 | −0.16 | 0.11 | 0.75 | −0.15 | −0.74 | 1.02 | 5.84 |
| SCML4 | AK093571 | 0.03 | 0.05 | 0.03 | 0.01 | 0.02 | 0.05 | 0.06 | 5.68 |
| RUNX2 | NM_001015051 | 0.46 | 0.42 | 0.39 | 2.96 | 0.10 | 0.53 | 1.86 | [symbol] |
| EDNRA | NM_001957 | 2.44 | 0.64 | 1.86 | 3.38 | 2.58 | 0.36 | 2.49 | 5.51 |
| ST6GAL2 | AB058780 | 0.00 | 0.02 | 0.56 | 2.16 | 0.00 | 0.01 | 0.38 | 5.50 |
| AF119913 | AF119913 | 1.10 | 1.47 | 1.17 | 3.86 | 1.41 | 1.11 | 0.84 | 5.44 |
| GJB2 | NM_004004 | 1.83 | 0.48 | 1.34 | 2.35 | 1.80 | 0.43 | 3.52 | 5.44 |
| AGAP2 | ENST00000257897 | 1.27 | −0.26 | 0.08 | 1.21 | 0.99 | 0.02 | −0.16 | 5.43 |
| PTPRG | BC036018 | 0.42 | 4.62 | 2.81 | 4.18 | 1.42 | 4.45 | 2.98 | 5.36 |
| B4GALNT3 | AK131277 | −0.03 | −0.48 | 0.03 | 1.45 | −0.22 | −0.34 | −0.24 | 5.17 |
| PRSS35 | NM_153362 | 0.25 | 0.79 | 0.74 | 1.35 | −0.05 | 0.48 | 1.01 | 5.15 |
| BMP8B | NM_001720 | 0.01 | 0.79 | 0.36 | 1.06 | −0.10 | 0.57 | 1.01 | [symbol] |
| A2M | NM_000014 | −0.02 | −0.68 | −0.01 | 0.57 | −0.09 | −0.72 | 0.74 | 5.13 |
| SYT12 | NM_177963 | 3.52 | 3.40 | 2.23 | −0.28 | 3.62 | 3.69 | 2.19 | 5.09 |
| IBSP | NM_004967 | 0.00 | 0.46 | 0.00 | 0.16 | 0.00 | 0.44 | 0.65 | [symbol] |
| MYOZ3 | NM_133371 | 2.14 | 2.91 | 2.38 | 3.74 | 1.68 | 2.91 | 2.50 | 4.93 |
| HEY1 | NM_012258 | 1.47 | −0.24 | −0.95 | 0.72 | −0.12 | −0.68 | 0.04 | 4.93 |
| PNOC | NM_006228 | −0.10 | 0.06 | 0.47 | 0.15 | 0.14 | 0.11 | 0.23 | 4.90 |
| BGLAP | NM_199173 | −0.66 | −0.06 | 0.34 | 1.55 | −0.61 | 0.34 | 0.05 | [symbol] |
| FGFR3 | NM_000142 | −2.22 | −0.95 | −2.72 | −1.78 | −1.93 | −1.19 | −2.73 | [symbol] |
| SMPD3 | NM_018667 | −0.42 | −0.85 | −1.67 | 0.75 | −0.60 | −1.98 | −1.64 | 4.79 |
| EPYC | NM_004950 | 0.00 | 0.01 | 0.00 | 1.01 | 0.00 | 0.31 | 0.01 | 4.77 |
| CYP26A1 | NM_057157 | 2.43 | 0.61 | 0.07 | 0.52 | 2.72 | 0.58 | 0.12 | 4.71 |
| MERTK | U08023 | 0.34 | 1.31 | 0.08 | 2.13 | 0.52 | 0.22 | 0.16 | 4.69 |
| CDH15 | NM_004933 | 4.78 | 0.16 | −0.80 | −0.29 | 3.50 | 1.00 | −0.11 | 4.64 |
| SLC29A1 | NM_004955 | 1.24 | 0.45 | 0.41 | 1.72 | 1.26 | 0.73 | 0.89 | 4.63 |
| TM7SF4 | NM_030788 | 0.01 | 0.02 | 0.08 | 0.99 | 0.26 | 0.11 | 0.67 | 4.62 |
| CPNE5 | NM_020939 | 1.35 | 0.20 | 1.16 | 1.63 | 1.18 | 1.55 | 2.07 | 4.54 |
| MGC4294 | BC002831 | 0.21 | −0.14 | −0.03 | 1.70 | −0.49 | −0.09 | 1.96 | 4.53 |
| CHRNA1 | NM_000079 | 0.09 | 0.09 | 1.55 | 0.10 | 0.39 | 2.50 | 1.68 | 4.36 |
| LOC645277 | XM_928321 | −0.09 | 0.18 | −0.01 | 0.52 | 0.07 | 0.41 | 0.43 | 4.34 |
| GZMA | NM_006144 | 0.32 | −0.01 | −0.03 | 0.46 | −0.01 | −0.05 | −0.01 | 4.34 |
| WFDC1 | NM_021197 | −0.50 | −1.02 | −1.08 | 2.09 | −0.24 | −1.68 | −1.55 | 4.23 |
| CHST1 | NM_003654 | 0.14 | 0.25 | 1.44 | −0.28 | −0.24 | 0.30 | 0.32 | 4.22 |
| C18ORFI | NM_181482 | 0.38 | 0.17 | 0.34 | 2.70 | 0.47 | 0.16 | 0.59 | 4.18 |
| DLXS | NM_005221 | −0.34 | 0.10 | −0.60 | 0.47 | −0.14 | −0.08 | −0.70 | [symbol] |
| NPTX2 | NM_002523 | −0.08 | −0.53 | 0.23 | 0.43 | −0.16 | −0.15 | −0.89 | 4.10 |
| MATN4 | NM_003833 | 0.82 | 1.22 | 1.80 | 6.16 | 0.63 | 1.63 | 1.64 | 4.09 |
| PLXNC1 | AB208934 | 1.72 | 0.13 | 0.21 | 1.53 | 0.94 | −0.17 | −0.06 | 4.06 |
| ACTG2 | NM_001615 | −0.18 | −0.33 | 0.03 | 1.02 | −0.10 | 0.36 | 0.34 | 4.01 |
| NKX3−2 | NM_001189 | 0.55 | −0.23 | −0.55 | −0.14 | 0.05 | 0.42 | −0.61 | [symbol] |
| LOC401022 | BC030713 | −0.38 | −0.80 | 0.58 | 0.64 | −0.27 | −0.84 | −0.32 | 3.98 |
| SEMA6A | NM_020796 | 0.04 | 0.45 | 0.13 | −0.09 | 0.01 | 0.33 | 0.58 | 3.97 |
| APCDDI | NM_153000 | −0.83 | 0.40 | −1.06 | −1.19 | −0.91 | −0.84 | −0.76 | 3.96 |
| TSPAN18 | NM_130783 | −1.05 | −1.04 | −0.55 | 1.27 | −0.87 | −0.78 | −0.64 | 3.95 |
| KLF12 | ENST00000377666 | 0.10 | 2.19 | 0.70 | 1.68 | 0.68 | 1.74 | 0.57 | 3.93 |
| GABRR1 | NM_002042 | −0.38 | −0.07 | −0.22 | 0.25 | −0.16 | 0.42 | 0.15 | 3.93 |
| HAPLN1 | NM_001884 | −0.99 | −0.41 | −1.86 | −0.74 | −0.80 | −0.89 | −1.98 | 3.88 |
| C9ORF110 | AK125961 | −0.59 | 0.48 | 0.22 | 0.85 | −0.85 | 0.37 | 0.49 | 3.87 |
| DERL3 | NM_198440 | 1.76 | −0.24 | −0.46 | 0.91 | 1.52 | 0.16 | −0.04 | 3.84 |
| DUSP2 | NM_004418 | −0.20 | −0.29 | 0.67 | 0.71 | 0.21 | 0.60 | 1.25 | 3.83 |
| PDK3 | BC038512 | 2.60 | 2.07 | 1.41 | 2.25 | 2.27 | 1.79 | 2.05 | 3.82 |
| SNTB1 | AF028828 | 1.17 | 2.57 | 0.18 | 1.62 | 2.10 | 2.49 | 0.10 | 3.79 |
| PDE9A | NM_002606 | −0.46 | −0.44 | −0.29 | −0.88 | −0.55 | −0.83 | −0.82 | 3.78 |
| RAMP1 | NM_005855 | −0.53 | −0.34 | −0.90 | 0.41 | 0.20 | −0.70 | 0.97 | 3.75 |
| C9ORF109 | AK127516 | −0.97 | −0.19 | −0.54 | −0.46 | −1.15 | −0.59 | −0.22 | 3.74 |
| ITGA8 | NM_003638 | −0.49 | −0.37 | −0.47 | −0.31 | −0.29 | −0.40 | −0.03 | 3.65 |
| CRYBA4 | NM_001886 | 0.05 | 1.03 | 0.30 | 0.98 | 0.10 | 0.42 | 1.42 | 3.63 |
| DYSF | NM_003494 | 2.43 | 0.53 | −0.98 | 1.02 | 2.45 | 0.57 | 0.96 | 3.63 |
| C1QTNF7 | NM_031911 | 0.09 | 0.67 | 0.24 | 2.07 | −0.02 | 0.66 | 1.01 | 3.60 |
| RASLIOA | NM_001007279 | −0.35 | −0.46 | 0.52 | 1.00 | −0.42 | −0.06 | −0.14 | 3.60 |
| BMP2 | NM_001200 | 1.78 | 3.47 | 1.04 | 1.12 | 1.33 | 2.96 | 0.74 | [symbol] |
| SGK223 | ENST00000330777 | −0.30 | 0.50 | 0.84 | 2.09 | −0.66 | 0.31 | 0.69 | 3.57 |
| UNC5B | NM_170744 | 0.40 | −0.69 | 0.26 | 1.01 | 0.26 | −0.57 | 0.77 | 3.57 |
| PLCH1 | NM_014996 | −0.02 | −0.01 | 0.00 | 0.10 | −0.02 | −0.01 | −0.01 | 3.53 |
| PODNL1 | THC2570737 | 0.45 | 1.19 | 1.36 | 2.27 | 0.42 | 0.91 | 2.03 | 3.52 |
| ZCCHC12 | NM_173798 | −0.09 | 0.42 | 1.82 | 0.07 | 0.13 | 0.24 | 0.50 | 3.51 |
| AOC2 | NM_001158 | 0.06 | −0.11 | 1.41 | 1.26 | −0.11 | 0.11 | 1.01 | 3.50 |
| PTK7 | NM_002821 | 0.26 | 0.39 | 0.58 | 1.92 | 0.07 | −0.03 | 0.89 | 3.49 |
| NET1 | NM_005863 | −0.46 | 0.15 | 0.54 | 1.81 | −0.46 | 0.11 | 1.44 | 3.43 |
| FAM198B | NM_016613 | −0.64 | −0.24 | 1.00 | 1.35 | −0.89 | −1.68 | 1.19 | 3.41 |
| LGALS2 | NM_006498 | −0.01 | 0.00 | 0.40 | 1.46 | −0.01 | −0.02 | 0.80 | 3.38 |
| OGDHL | NM_018245 | 0.86 | −0.27 | −0.16 | −0.19 | 0.27 | −0.15 | −0.27 | 3.37 |
| BMP8B | NM_181809 | −0.21 | −0.59 | −0.60 | −0.30 | 0.97 | −0.39 | −0.55 | [symbol] |
| TRIB1 | NM_025195 | 1.40 | 1.79 | −0.30 | 0.09 | 1.14 | 1.95 | 0.73 | 3.35 |
| LOC645722 | XM_944447 | 0.18 | 0.86 | 0.57 | 1.80 | 0.53 | 1.24 | 0.79 | 3.29 |
| RUNX1 | NM_001001890 | 1.20 | 1.68 | 0.95 | 1.82 | 1.18 | 1.52 | 1.69 | 3.27 |
| FAM78A | NM_033387 | −0.78 | −0.76 | −0.72 | 1.68 | −0.79 | −0.42 | −0.75 | 3.27 |
| SATB2 | AK025127 | 0.26 | 0.65 | 0.28 | 0.59 | 0.05 | 1.26 | 0.06 | [symbol] |
| ALPL | NM_000478 | −0.15 | −2.46 | −0.66 | −0.34 | −0.29 | −1.34 | −1.21 | [symbol] |
| CRYBB1 | NM_001887 | 0.21 | −0.25 | −0.34 | −0.25 | −0.35 | 0.06 | 0.90 | 3.23 |
| PELI2 | NM_021255 | 0.79 | 0.20 | 0.61 | 0.83 | 0.91 | 0.35 | −0.67 | 3.23 |
| TBX2 | NM_005994 | −0.92 | 0.03 | −0.75 | 0.21 | 0.31 | 0.39 | 0.17 | 3.20 |
| DYNC1I1 | NM_004411 | 0.05 | 0.15 | −1.69 | 0.04 | −0.08 | −0.57 | −1.65 | 3.18 |
| SFRS13B | AK090803 | 0.09 | −0.02 | −0.16 | 1.45 | 0.86 | 0.01 | 0.09 | 3.12 |
| MDK | NM_001012334 | 0.85 | 1.61 | 0.76 | 1.97 | 0.77 | 1.26 | 0.32 | 3.09 |
| VASH1 | NM_014909 | 0.00 | 0.77 | 0.99 | 1.83 | −0.06 | 0.50 | 1.09 | 3.04 |
| ABCA4 | NM_000350 | 0.54 | −0.10 | −0.17 | 0.17 | 0.23 | −0.04 | 0.17 | 3.02 |
| LIMD2 | NM_030576 | −1.19 | −0.11 | −0.97 | 0.89 | −1.02 | −0.22 | −0.26 | 2.99 |
| PTPRZ1 | NM_002851 | −0.08 | 0.10 | −0.28 | −0.18 | −0.69 | −0.30 | 0.38 | 2.98 |
| LOC728715 | BC039117 | 0.27 | 0.31 | 0.12 | 0.32 | 0.77 | 0.53 | 0.43 | 2.97 |
| BMP7 | ENST00000371291 | −0.57 | −0.65 | 1.31 | −0.26 | −0.17 | −0.32 | 0.29 | [symbol] |
| MYL10 | BC002778 | −0.08 | 0.17 | −0.03 | 0.00 | −0.12 | 0.12 | 0.72 | 2.94 |
| LINGO1 | NM_032808 | −1.71 | −1.25 | 0.06 | 0.75 | −1.44 | −0.57 | 0.56 | 2.89 |
| LGR6 | NM_001017403 | −0.01 | 0.04 | 0.00 | −0.06 | −0.01 | 0.02 | 0.04 | 2.89 |
| NES | NM_006617 | 0.87 | 1.06 | 0.83 | 0.78 | 0.55 | 0.89 | 0.79 | 2.88 |
| FGF23 | NM_020638 | −0.11 | 0.31 | −0.01 | −0.07 | −0.15 | 0.17 | 0.27 | 2.87 |
| MATN2 | NM_030583 | 0.38 | −0.09 | −0.33 | 0.29 | 0.19 | −0.63 | −0.12 | 2.86 |
| C15ORF28 | AK021784 | 0.27 | 0.19 | 0.01 | 1.40 | 0.18 | 0.43 | 0.08 | 2.86 |
| COL9A2 | NM_001852 | −0.02 | 0.09 | 1.14 | 0.75 | 0.08 | 0.19 | 0.35 | [symbol] |
| SHROOM1 | NM_133456 | −0.01 | 0.52 | 0.46 | 1.80 | 0.10 | 0.71 | 0.61 | 2.84 |
| SH3PXD2B | NM_001017995 | 0.99 | 0.46 | 0.61 | 1.30 | 0.85 | 0.34 | 0.39 | 2.82 |
| LONRF2 | NM_198461 | −0.24 | 0.20 | 0.07 | −0.28 | −0.25 | −0.12 | 0.30 | 2.82 |
| AF116642 | AF116642 | 0.03 | 1.47 | 0.43 | 0.87 | −0.02 | 1.60 | 0.36 | 2.80 |
| TCF4 | AK021980 | 0.01 | 0.30 | 0.71 | 1.19 | 0.21 | 0.56 | 0.75 | 2.72 |
| GPR84 | NM_020370 | 0.06 | 0.16 | 0.50 | 1.60 | 0.84 | 0.60 | 0.44 | 2.72 |
| LOC641518 | BC020624 | −0.01 | 0.00 | −0.01 | −0.01 | 0.00 | 0.00 | 0.00 | 2.60 |
| PTPDC1 | NM_177995 | 1.33 | −0.11 | −0.13 | 1.37 | 0.96 | −0.10 | 0.31 | 2.60 |
| MICALL2 | NM_182924 | 0.36 | 0.32 | 0.63 | 1.32 | −0.07 | −0.07 | 0.66 | 2.59 |
| ARAP3 | NM_022481 | 1.34 | 2.08 | 1.14 | 1.17 | 0.83 | 1.67 | 1.28 | 2.55 |
| SPRY4 | NM_030964 | −1.03 | −0.55 | −1.58 | −1.18 | 0.17 | −0.19 | −1.35 | 2.52 |
| PRDM6 | ENST00000261364 | −0.32 | −0.31 | 0.06 | 0.99 | −0.41 | −0.34 | −0.87 | 2.52 |
| AKAP7 | NM_016377 | −0.11 | 0.09 | 0.14 | 0.59 | −0.35 | −0.39 | 0.34 | 2.51 |
| GAS2L3 | BX649059 | 2.35 | −0.07 | 0.01 | 0.73 | 2.29 | 0.09 | 0.64 | 2.43 |
| SDC1 | NM_001006946 | −0.01 | 0.02 | −0.64 | 0.73 | −0.04 | −0.42 | 0.08 | 2.42 |
| HIVEP3 | ENST00000372583 | −0.85 | 0.58 | −0.54 | 0.01 | −0.99 | 0.42 | −0.32 | 2.40 |
| CNIH3 | NM_152495 | 0.43 | −0.71 | −0.92 | −0.16 | 0.74 | −0.04 | −0.06 | 2.38 |
| DUSP13 | NM_001007271 | −0.25 | −0.68 | −0.46 | −0.25 | −0.03 | −0.49 | −0.38 | 2.37 |
| DLL1 | NM_005618 | 0.73 | 0.08 | −0.41 | −0.09 | 1.16 | 0.12 | −0.31 | 2.36 |
| RSU1 | BC008384 | 0.26 | 0.09 | 0.00 | 1.01 | −0.04 | 0.18 | 0.08 | 2.33 |
| PTHLH | ENST00000354417 | −0.18 | 1.90 | −0.76 | −0.12 | 0.53 | 1.92 | −0.44 | [symbol] |
| C20ORF200 | NM_152757 | 0.03 | 0.56 | 1.02 | 0.34 | 0.02 | 0.19 | 0.05 | 2.30 |
| SRPX2 | NM_014467 | 0.17 | 0.56 | −0.32 | 0.40 | −0.03 | −0.04 | 0.04 | 2.29 |
| MATN3 | NM_002381 | 0.61 | −0.79 | −0.78 | −0.11 | 0.06 | −0.80 | −0.79 | [symbol] |
| EFR3B | AF131834 | −0.36 | −0.51 | −0.42 | 0.36 | −0.83 | −0.82 | 0.21 | 2.28 |
| ZNF609 | NM_015042 | 0.27 | 1.14 | 2.24 | 0.94 | 0.79 | 1.81 | 0.93 | 2.27 |
| FAM101B | NM_182705 | 0.26 | −1.26 | −1.06 | 0.41 | 0.83 | −0.85 | −0.30 | 2.27 |
| HCG1818231 | XM_001131389 | 2.13 | 1.59 | 1.02 | 0.80 | 2.22 | 1.81 | 0.39 | 2.25 |
| GFI1 | NM_005263 | −0.04 | 0.23 | 0.12 | −0.19 | −0.11 | −0.01 | 0.31 | 2.25 |
| TSPAN6 | NM_003270 | 0.16 | 0.98 | 1.13 | 1.17 | −0.08 | 0.62 | 0.50 | 2.24 |
| GRAMDIC | NM_017577 | 0.03 | 0.20 | 0.07 | 3.38 | 0.00 | 0.32 | 0.37 | 2.20 |
| NOTCH1 | NM_017617 | −0.42 | −0.31 | −0.60 | −0.80 | −0.89 | −0.93 | −0.30 | 2.19 |
| CMTM8 | NM_178868 | 0.09 | −0.70 | −1.55 | −0.28 | 0.32 | 0.27 | −1.46 | 2.13 |
| PECAM1 | NM_000442 | 1.94 | 0.15 | −2.05 | −1.26 | 1.82 | 0.51 | −1.52 | 2.08 |
| SEMA3A | NM_006080 | −1.54 | −3.11 | −2.12 | −0.20 | −1.49 | −2.72 | −1.73 | 2.05 |
| DLX4 | NM_138281 | 0.44 | 0.29 | −1.72 | −0.83 | 0.03 | 0.20 | −1.96 | 2.05 |
| ARHGEF19 | NM_153213 | 0.51 | 1.48 | −0.06 | 0.75 | 0.62 | 1.22 | 0.64 | 2.04 |
| PLEKHG2 | NM_022835 | −0.17 | 0.17 | −0.37 | 0.91 | −0.24 | 0.09 | −0.23 | 2.01 |
| C8ORFK29 | AB196634 | 0.38 | −0.19 | 0.00 | 0.86 | −0.19 | 0.26 | 0.02 | 1.98 |
| FAM105A | NM_019018 | 0.12 | 0.23 | −0.88 | −0.97 | 0.65 | 0.04 | −0.90 | 1.95 |
| DLX3 | NM_005220 | −0.85 | −0.20 | −1.70 | −1.25 | −1.27 | −1.46 | −1.09 | 1.95 |
| MGAT5B | NM_144677 | −0.07 | −0.25 | −0.03 | 0.48 | 0.06 | 0.11 | −0.27 | 1.92 |
| TMEM44 | NM_138399 | 0.94 | 0.78 | 0.25 | 0.84 | 0.49 | 0.63 | 0.37 | 1.88 |
| F12 | NM_000505 | 1.32 | 0.98 | 0.48 | −0.26 | 1.25 | 1.14 | 0.83 | 1.87 |
| NFYA | AK002098 | −0.59 | 0.20 | −0.01 | 0.73 | −0.51 | 0.11 | 0.08 | 1.84 |
| CDKN2B | NM_078487 | 1.26 | 1.22 | 0.35 | −0.41 | 1.09 | 0.98 | −0.47 | 1.78 |
| KCNAB1 | NM_003471 | −0.16 | −0.08 | 0.33 | −0.23 | −0.19 | −0.13 | 0.03 | 1.77 |
| PGAM2 | NM_000290 | −0.98 | −0.35 | −0.76 | −0.29 | −1.04 | −0.56 | −0.18 | 1.77 |
| SHTSA2 | NM_001007538 | −2.02 | −2.19 | −2.22 | −2.46 | −2.03 | −1.44 | −1.27 | 1.77 |
| WDR33 | NM_018383 | −0.15 | −0.09 | −0.10 | 2.87 | −0.16 | 0.00 | −0.10 | 1.76 |
| TMEM155 | NM_152399 | −0.90 | −2.19 | −2.78 | −0.82 | −1.26 | −1.72 | −3.11 | 1.75 |
| RCC2 | NM_018715 | −0.88 | 0.25 | 0.06 | 0.62 | −0.82 | 0.08 | 0.14 | 1.74 |
| KCNQ2 | NM_172109 | 0.03 | 0.16 | 0.07 | −0.03 | 0.01 | 0.14 | 0.21 | 1.74 |
| MAST4 | NM_198828 | 0.03 | −0.15 | 0.31 | 0.64 | 0.09 | 0.28 | 0.20 | 1.74 |
| ZDHHC23 | NM_173570 | −0.68 | −0.62 | −1.66 | −0.72 | −0.68 | −0.99 | −1.10 | 1.73 |
| LZTS1 | NM_021020 | −2.61 | −1.02 | −0.85 | −0.37 | −2.46 | −1.15 | −0.56 | 1.72 |
| MMP14 | NM_004995 | 0.63 | −0.23 | −0.54 | −0.31 | 0.43 | 0.08 | −0.04 | [symbol] |
| ARHGAP32 | NM_014715 | 1.14 | 0.97 | 0.75 | 0.45 | 0.79 | 0.48 | 0.66 | 1.69 |
| LRRC1 | ENST00000370892 | −0.01 | 0.00 | 0.03 | 0.63 | −0.02 | 0.13 | 0.00 | 1.68 |
| PLK2 | NM_006622 | −0.66 | −0.80 | −0.47 | 0.41 | −0.77 | −0.81 | −1.04 | 1.68 |
| INPPL1 | NM_001567 | −0.05 | 0.39 | 0.02 | 0.50 | −0.16 | 0.23 | 0.04 | 1.68 |
| TXNDC3 | NM_016616 | 0.03 | 0.15 | 0.07 | 0.44 | 0.01 | 0.12 | 0.19 | 1.68 |
| KIFAP3 | NM_014970 | −0.01 | 0.09 | 0.37 | 0.29 | −0.41 | −0.51 | −0.33 | 1.67 |
| FAT3 | ENST00000298047 | 0.07 | −0.13 | −0.09 | −0.14 | 0.05 | −0.15 | −0.13 | 1.67 |
| CHTF18 | NM_022092 | −0.09 | −0.23 | −0.64 | 0.59 | 0.04 | 0.42 | 0.27 | 1.66 |
| C17ORF60 | ENST00000332935 | 1.79 | 1.00 | −1.40 | −0.49 | 1.55 | 0.61 | −0.51 | 1.66 |
| COL8A1 | THC2501739 | −0.20 | −1.42 | −1.11 | 0.45 | −0.26 | −2.09 | −0.85 | 1.65 |
| LRCH1 | CB051804 | 0.48 | 0.44 | 0.18 | 0.60 | 0.94 | 0.43 | 0.49 | 1.63 |
| SRrp35 | NM_080743 | 0.01 | −0.01 | −0.01 | 0.38 | 0.90 | −0.01 | −0.01 | 1.63 |
| ETV6 | NM_001987 | 1.45 | 0.06 | −0.19 | 0.49 | 1.97 | −0.17 | 0.09 | 1.61 |
| CKM | NM_001824 | −0.19 | −0.10 | −0.48 | 0.10 | −0.20 | −0.36 | −0.27 | 1.60 |
| KLHDC8A | NM_018203 | 0.00 | 0.07 | 0.04 | 0.03 | −0.02 | −0.04 | 0.08 | 1.57 |
| SEPN1 | NM_020451 | −0.26 | −0.17 | −0.38 | 0.17 | −0.35 | −0.63 | −0.60 | 1.56 |
| CMTM1 | NM_052999 | 0.63 | −0.33 | −0.03 | −0.29 | −0.08 | −0.37 | −0.34 | 1.55 |
| HVCN1 | NM_032369 | 0.83 | 0.23 | −1.16 | −0.66 | 0.59 | −0.84 | −1.50 | 1.54 |
| SPREDI | NM_152594 | 0.34 | −0.36 | −0.48 | −0.24 | 0.44 | −0.39 | 0.08 | 1.54 |
| SPHK1 | NM_021972 | 0.27 | 0.19 | −1.32 | −0.43 | 1.04 | 0.17 | −0.45 | 1.53 |
| ANKH | NM_054027 | 0.51 | −0.65 | −0.69 | −0.94 | 0.86 | −0.37 | −0.31 | [symbol] |
| DOCK4 | NM_014705 | −0.67 | −0.19 | −0.40 | −0.97 | −0.65 | −0.82 | −0.56 | 1.47 |
| C20ORF160 | NM_080625 | 0.10 | 0.60 | −0.01 | −0.07 | −0.02 | 0.00 | 0.02 | 1.44 |
| SEMA3D | NM_152754 | −0.38 | −2.39 | −3.06 | −1.61 | −0.50 | −2.99 | −2.57 | 1.41 |
| MLLT1I | NM_006818 | 0.25 | −0.06 | −0.86 | −0.23 | 0.40 | −0.32 | −0.40 | 1.39 |
| MYB | NM_005375 | −1.66 | −1.81 | −2.51 | −3.36 | −1.30 | −1.61 | −1.85 | 1.38 |
| SGIP1 | NM_032291 | 1.49 | −0.74 | −0.56 | −0.11 | 1.58 | −0.07 | −0.48 | 1.37 |
| TRPC3 | NM_003305 | −0.46 | −0.26 | −2.04 | −1.85 | −0.01 | −0.63 | −1.74 | 1.33 |
| RAI14 | NM_015577 | 0.57 | 0.81 | −0.08 | 0.23 | 0.80 | 0.56 | 0.31 | 1.32 |
| ZNF642 | NM_198494 | −1.16 | −0.59 | −1.93 | −0.53 | −1.19 | −1.55 | −1.68 | 1.32 |
| ACAP3 | NM_030649 | 0.40 | 0.24 | 0.10 | 0.21 | −0.03 | −0.17 | −0.11 | 1.29 |
| RIN2 | NM_018993 | −0.24 | −0.17 | 0.08 | 0.16 | 0.15 | −0.20 | 0.07 | 1.29 |
| CSRNP2 | NM_030809 | −0.13 | 0.33 | −0.05 | 0.24 | 0.05 | −0.09 | −0.30 | 1.29 |
| CMTM3 | NM_144601 | 0.20 | 0.35 | 0.37 | −0.10 | −0.17 | −0.31 | 0.23 | 1.28 |
| EML4 | NM_019063 | −1.02 | −0.19 | −0.26 | 0.25 | −0.67 | −0.41 | −0.57 | 1.27 |
| MAGED4B | NM_030801 | 0.70 | 0.98 | 0.37 | 0.03 | 0.12 | 0.37 | −0.22 | 1.26 |
| HISTIH4K | NM_003541 | 0.04 | 0.76 | −0.18 | −0.06 | 0.14 | 0.74 | −0.15 | 1.26 |
| CELSR2 | NM_001408 | 0.04 | −0.51 | −0.52 | −0.10 | −0.43 | −0.39 | −0.30 | 1.24 |
| ID3 | NM_002167 | −0.64 | 0.05 | −0.17 | −0.25 | −0.66 | −0.17 | −0.44 | [symbol] |
| HOMER2 | NM_199330 | −0.49 | −1.49 | −2.36 | −2.59 | −0.65 | −2.83 | −1.04 | 1.23 |
| HOOK3 | BC013679 | 0.20 | −1.00 | −1.54 | 0.11 | 0.14 | −1.60 | −1.29 | 1.21 |
| SEMA7A | NM_003612 | −0.25 | −2.18 | −1.08 | −1.07 | −0.05 | −1.97 | −0.53 | 1.19 |
| TMEM169 | NM_138390 | 0.43 | 0.03 | 0.44 | 0.00 | 0.17 | 0.19 | 0.03 | 1.17 |
| LOC402778 | ENST00000382123 | −0.70 | −0.33 | −0.44 | −2.84 | −0.80 | −1.11 | −1.74 | 1.16 |
| TPST2 | NM_001008566 | −0.10 | −0.63 | −1.25 | −1.02 | −0.12 | −0.92 | −0.93 | 1.15 |
| CDKL5 | NM_003159 | 1.25 | −0.53 | −0.64 | −0.21 | 1.32 | −0.51 | −0.89 | 1.13 |
| PI4KAP2 | NM_199345 | 0.56 | −0.51 | −0.81 | −0.08 | 0.17 | −0.79 | −0.58 | 1.13 |
| FAM100B | NM_182565 | 0.98 | 0.41 | −0.56 | −0.15 | 1.22 | 0.59 | −0.42 | 1.12 |
| CENPP | ENST00000375587 | −1.32 | −0.70 | −0.38 | −0.36 | −1.28 | −1.02 | −0.53 | 1.12 |
| ST3GAL1 | NM_003033 | 0.68 | 0.37 | −0.43 | −0.26 | 1.11 | 0.49 | −0.31 | 1.09 |
| PPARD | NM_006238 | 0.01 | −0.38 | −0.49 | −0.30 | 0.31 | −0.36 | −0.36 | 1.07 |
| FKBP10 | NM_021939 | 0.32 | −0.26 | −0.38 | 0.00 | 0.19 | −0.37 | −0.46 | 1.06 |
| TRAF3IP3 | NM_025228 | −0.37 | −0.08 | −0.30 | −0.46 | 0.00 | −0.20 | −0.11 | 1.05 |
| CBFB | NM_001755 | −0.76 | −0.69 | −0.61 | −0.25 | −0.16 | −0.98 | −0.48 | [symbol] |
| KIAA1211 | AL133028 | −0.11 | −0.64 | −0.02 | −0.63 | −0.37 | −0.67 | −0.65 | 1.04 |
| FGFR1 | NM_023110 | 1.00 | −0.14 | 0.01 | −0.56 | 0.97 | −0.23 | −0.04 | [symbol] |
| RTEL1 | NM_016434 | −0.32 | −1.00 | −1.23 | −0.06 | −0.53 | −0.87 | −0.38 | 1.00 |
| BAIAP2 | NM_017451 | −1.11 | −1.69 | −1.99 | −0.93 | −0.77 | −1.57 | −1.80 | 1.00 |
| PHTF2 | NM_020432 | −0.13 | −0.67 | −1.02 | −0.87 | −0.45 | −1.07 | −0.34 | 0.98 |
| ITM2C | NM_030926 | 0.31 | 0.08 | −0.34 | −1.20 | 0.10 | −0.93 | −1.26 | 0.98 |
| OSBPL5 | NM_020896 | 0.94 | 0.02 | −0.66 | −0.31 | 0.64 | −0.18 | −0.54 | 0.98 |
| PC | NM_000920 | 2.33 | 0.75 | −0.39 | −0.29 | 2.35 | 0.66 | −0.29 | 0.98 |
| RAB31 | NM_006868 | 0.22 | −0.27 | −0.72 | −1.00 | 0.25 | −0.46 | −0.65 | 0.96 |
| KIAA1217 | NM_019590 | 0.19 | −0.93 | −0.87 | −0.51 | −0.15 | −1.56 | −1.23 | [symbol] |
| ERG | NM_004449 | 0.22 | −1.03 | −0.91 | −0.34 | 0.34 | −0.93 | −0.39 | 0.95 |
| SMAD7 | NM_005904 | 0.79 | 0.12 | −0.89 | −1.20 | 1.08 | 0.05 | −0.60 | 0.89 |
| ZNF48 | NM_152652 | −1.42 | −1.19 | −1.77 | −0.49 | −1.24 | −0.49 | −1.48 | 0.88 |
| IL21R | NM_181078 | −0.04 | −1.13 | −1.65 | −1.81 | −0.20 | −0.55 | −1.29 | 0.87 |
| C16ORF93 | NM_001014979 | −0.39 | −0.91 | −1.77 | −0.23 | −0.34 | −0.70 | −0.86 | 0.86 |
| EHD3 | NM_014600 | −1.07 | −0.01 | −0.89 | −0.55 | −1.33 | −0.75 | −0.58 | 0.85 |
| DNMT3B | NM_175850 | −0.79 | −0.20 | −1.28 | −0.48 | −0.64 | −0.13 | −0.91 | 0.80 |
| SLC20A1 | NM_005415 | −2.35 | −1.04 | −0.77 | −0.29 | −1.65 | −0.60 | −0.30 | 0.80 |
| PSRC1 | NM_032636 | 1.00 | −0.17 | −0.63 | −0.47 | 0.91 | 0.09 | −0.36 | 0.78 |
| LOC653464 | XM_209227 | 0.83 | 0.05 | −0.03 | −0.40 | 0.93 | −0.17 | −0.26 | 0.78 |
| DOT1L | BC032803 | −0.05 | −0.69 | −0.60 | −0.31 | −0.13 | −0.22 | −0.32 | 0.77 |
| CYTSB | NM_001033553 | 0.34 | −0.98 | −1.11 | −0.77 | 0.21 | −0.92 | −0.73 | 0.76 |
| EPHA2 | NM_004431 | −0.11 | −2.05 | −1.74 | −1.71 | 1.03 | −2.56 | −2.64 | 0.74 |
| CHD9 | NM_025134 | 0.72 | −0.72 | −1.12 | −0.47 | 0.76 | −0.95 | −1.09 | 0.73 |
| ATAD3B | NM_031921 | −1.24 | −0.73 | −0.98 | −0.57 | −1.02 | −1.04 | −0.56 | 0.72 |
| SLC5A6 | NM021095 | −1.10 | −0.11 | −1.20 | −0.62 | −1.02 | −0.45 | −0.95 | 0.71 |
| MB | NM_203377 | −0.96 | −1.29 | −1.47 | −1.41 | −0.87 | −1.23 | −1.21 | 0.64 |
| CEP152 | NM_014985 | 1.58 | 0.29 | −1.94 | −1.20 | 1.44 | 0.33 | −2.15 | 0.61 |
| GTF3C1 | NM_001520 | −0.60 | −0.65 | −0.91 | −0.64 | −0.62 | −1.10 | −1.00 | 0.60 |
| PRR7 | NM_030567 | 1.19 | 0.37 | −0.75 | −1.01 | 1.24 | 0.55 | −0.94 | 0.58 |
| NCAPD2 | NM_014865 | 0.14 | 0.25 | −0.57 | −0.64 | −0.12 | 0.09 | −0.60 | 0.57 |
| STX18 | NM_016930 | −0.11 | −0.64 | −0.88 | −0.55 | −0.17 | −1.03 | −1.10 | 0.55 |
| DAB2IP | NM_032552 | 1.74 | −0.09 | −0.10 | −0.60 | 2.03 | −0.25 | −0.60 | 0.54 |
| MSX1 | NM_002448 | 0.07 | −0.86 | −0.86 | −0.58 | −0.17 | −1.16 | −1.47 | [symbol] |
| RECQL4 | NM_004260 | −0.52 | −0.53 | −1.61 | −0.95 | −0.50 | −0.32 | −0.78 | 0.53 |
| SPC24 | NM_182513 | 0.07 | −1.39 | −3.06 | −1.92 | 0.15 | −1.38 | −2.55 | 0.48 |
| LOC100288737 | CN479126 | −1.09 | −1.23 | −1.78 | −1.34 | −1.10 | −1.39 | −2.39 | 0.45 |
| FAM72D | NM_207418 | 0.29 | 0.00 | −1.25 | −1.95 | 0.26 | −0.17 | −1.30 | 0.45 |
| CTSC | NM_148170 | −1.81 | −0.98 | −2.12 | −1.98 | −1.57 | −1.70 | −1.55 | 0.41 |
| MRAS | NM_012219 | −1.37 | −0.86 | −1.17 | −0.82 | −1.18 | −1.28 | −1.32 | 0.39 |
| KCTD5 | NM_018992 | 0.65 | −0.07 | −0.62 | −0.74 | 0.69 | −0.07 | −0.69 | 0.37 |
| SPTBNS | NM_016642 | −1.10 | −2.83 | −2.69 | −1.06 | −1.27 | −2.27 | −2.53 | 0.35 |
| RASAL2 | NM_170692 | −0.33 | −0.54 | −0.89 | −0.74 | −0.51 | −0.93 | −0.96 | 0.32 |
| NFKBIL2 | NM_013432 | −0.85 | −0.85 | −2.48 | −1.36 | −1.12 | −0.70 | −1.64 | 0.32 |
| PTPN14 | NM_005401 | −0.50 | −0.32 | −1.29 | −0.75 | −0.51 | −0.13 | −0.88 | 0.30 |
| FGFI | NM_000800 | −1.98 | −4.30 | −4.88 | −4.27 | −1.03 | −4.64 | −3.40 | 0.28 |
| TMTC4 | NM_032813 | −1.48 | −0.26 | −1.26 | −1.21 | −1.00 | −0.65 | −0.85 | 0.27 |
| SMARCB1 | NM_003073 | 0.02 | −1.11 | −1.17 | −1.19 | −0.28 | −1.67 | −2.28 | 0.25 |
| TP53111 | AF010315 | −0.61 | −0.54 | −0.73 | −1.70 | −0.43 | −0.81 | −0.78 | 0.25 |
| CALM1 | NM_006888 | −0.80 | −1.33 | −1.24 | −1.11 | −0.77 | −1.56 | −1.51 | 0.24 |
| FZD7 | NM_003507 | −0.90 | −0.19 | −0.85 | −1.09 | −0.97 | −0.62 | −1.00 | 0.21 |
| KIF4A | NM_012310 | 0.53 | −0.78 | −2.92− | 3.26 | 0.27 | −0.58 | −1.69 | 0.20 |
| RPL27A | ENST00000356931 | −1.69 | −2.20 | −1.91 | −2.59 | −2.15 | −1.91 | −2.62 | 0.20 |
| BAMBI | NM_012342 | −1.92 | −2.24 | −2.22 | −1.73 | −1.29 | −2.11 | −2.69 | 0.20 |
| MIIP | NM_021933 | −0.69 | −0.68 | −1.15 | −1.01 | −0.65 | −1.28 | −1.15 | 0.19 |
| IER2 | NM_004907 | 0.56 | −0.44 | −1.12 | −0.96 | 0.57 | −0.16 | −1.33 | 0.14 |
| RTN2 | NM_005619 | 0.94 | 0.33 | −0.48 | −0.92 | 0.55 | −0.02 | −1.43 | 0.11 |
| LMNB1 | NM_005573 | −1.58 | −2.48 | −4.55 | −2.68 | −1.72 | −2.07 | −2.54 | 0.08 |
| KIF22 | NM_007317 | 0.12 | −0.67 | −2.06 | −1.62 | 0.17 | −0.44 | −1.23 | 0.08 |
| TACC3 | NM_006342 | −0.56 | −0.96 | −3.85 | −2.56 | −0.60 | −0.64 | −2.39 | 0.00 |
| DUSP10 | NM_007207 | −0.50 | −1.54 | −2.76 | −2.51 | −0.65 | −0.91 | −3.16 | −0.01 |
| TEAD4 | NM_003213 | −1.34 | −1.28 | −2.04 | −1.30 | −1.25 | −1.10 | −1.25 | [symbol] |
| C16ORF59 | NM_025108 | −1.67 | −1.24 | −2.80 | −1.98 | −1.59 | −0.89 | −1.68 | −0.06 |
| TAGLN | NM_001001522 | −0.88 | −4.37 | −4.38 | −2.43 | −0.93 | −4.88 | −4.08 | −0.10 |
| HPCAL1 | NM_134421 | −0.29 | −0.44 | −1.06 | −2.05 | −0.49 | −0.98 | −1.46 | −0.15 |
| FANCA | NM_000135 | −2.26 | −1.86 | −3.10 | −2.49 | −1.91 | −1.69 | −1.76 | −0.16 |
| PVRL2 | NM_002856 | 0.10 | −0.67 | −1.85 | −1.48 | 0.40 | −1.10 | −1.87 | −0.17 |
| CDC25C | NM_001790 | 1.35 | −1.12 | −2.98 | −3.25 | 1.26 | −0.83 | −2.95 | −0.17 |
| BTBD3 | NM_014962 | −0.55 | −0.40 | −1.06 | −1.42 | −0.47 | −0.58 | −1.24 | −0.21 |
| AMMECR1 | NM_015365 | −0.89 | −0.46 | −0.97 | −1.36 | −1.30 | −1.16 | −1.38 | −0.27 |
| CORT | NM_001302 | −0.73 | −0.98 | −1.80 | −1.33 | −0.60 | −1.03 | −1.83 | −0.31 |
| ZWILCH | NM_017975 | −0.91 | −0.80 | −2.02 | −1.69 | −0.76 | −1.06 | −1.43 | −0.35 |
| QSER1 | NM_001076786 | −0.39 | −1.10 | −1.32 | −1.39 | 0.02 | −1.29 | −1.54 | −0.36 |
| PACSIN2 | NM_007229 | 0.70 | −0.41 | −0.96 | −1.41 | 0.58 | −0.82 | −1.87 | −0.37 |
| ECEL1 | NM_004826 | 0.37 | −1.86 | −3.07 | −3.17 | 0.21 | −2.44 | −2.98 | −0.41 |
| ID1 | NM_002165 | −1.05 | −1.02 | −1.64 | −2.28 | −1.20 | −1.06 | −2.18 | [symbol] |
| CENPN | BC039021 | −0.91 | −1.04 | −2.39 | −1.58 | −0.92 | −1.04 | −1.85 | −0.42 |
| TIMELESS | NM_003920 | −1.13 | −0.89 | −2.75 | −1.78 | −1.12 | −0.87 | −1.60 | −0.48 |
| LRWD1 | NM_152892 | −2.00 | −1.31 | −2.10 | −1.65 | −1.65 | −1.39 | −2.13 | −0.57 |
| SLC20A2 | NM_006749 | −1.98 | −1.88 | −2.33 | −1.94 | −1.63 | −1.96 | −2.39 | −0.65 |
| CASC5 | NM_170589 | 0.23 | −0.79 | −2.72 | −3.14 | −0.08 | −1.80 | −2.59 | −0.67 |
| TPM2 | NM_213674 | −0.53 | −1.19 | −1.57 | −1.84 | −0.35 | −1.73 | −1.85 | −0.69 |
| TPM1 | NM_001018004 | −2.10 | −2.48 | −2.22 | −2.09 | −1.85 | −3.12 | −2.27 | −0.72 |
| SS18L1 | NM_198935 | −0.19 | −1.51 | −2.65 | −1.97 | 0.60 | −1.66 | −2.36 | −0.72 |
| GPRIN1 | NM_052899 | −1.00 | −0.17 | −2.44 | −2.82 | −0.91 | −0.65 | −2.30 | −0.74 |
| ACTN4 | NM_004924 | −0.69 | −1.36 | −1.56 | −1.96 | −0.76 | −1.94 | −2.25 | −0.74 |
| SLC31A2 | NM_001860 | −0.98 | −1.46 | −2.44 | −2.62 | −0.80 | −1.68 | −2.16 | −0.77 |
| BLM | NM_000057 | −0.38 | −1.31 | −2.86 | −3.22 | −0.40 | −1.35 | −2.02 | −0.84 |
| CDC25A | NM_001789 | −3.72 | −1.74 | −3.93 | −2.67 | −3.41 | −1.60 | −3.10 | −0.96 |
| ACOT7 | NM_181864 | −0.72 | −1.31 | −2.58 | −2.69 | −0.78 | −1.77 | −2.43 | −1.02 |
| KRAS | NM_033360 | −1.27 | −0.86 | −1.71 | −2.39 | −0.77 | −1.31 | −2.42 | −1.03 |
| AURKB | NM_004217 | −0.13 | −1.31 | −2.87 | −3.61 | 0.16 | −0.99 | −2.78 | −1.11 |
| DIAPH3 | NM_001042517 | −0.30 | −1.56 | −3.75 | −3.14 | −0.11 | −0.89 | −3.35 | −1.12 |
| ATOH8 | NM_032827 | −1.61 | −1.54− | 2.86 | −2.80 | −2.04 | −1.99 | −3.78 | −1.16 |
| CKAP2L | NM_152515 | 0.37 | −1.44 | −3.68 | −4.25 | 0.15 | −1.35 | −4.26 | −1.18 |
| ERCC6L | NM_001009954 | −0.45 | −1.38 | −3.50 | −3.87 | −0.26 | −1.22 | −3.83 | −1.30 |
| CENPI | NM_006733 | −0.80 | −2.93 | −2.64 | −2.82 | −0.94 | −2.20 | −2.91 | −1.35 |
| CDC6 | NM_001254 | 0.53 | −1.90 | −5.80 | −4.06 | 0.51 | −2.29 | −3.41 | −1.46 |
| DUSP5 | NM_004419 | 0.77 | −0.65 | −2.77 | −3.76 | 1.71 | −0.30 | −3.05 | −1.58 |
| HUS1 | NM_004507 | −0.11 | −1.58 | −2.71 | −2.77 | −0.01 | −1.88 | −3.50 | −1.65 |
| OXCT2 | NM_022120 | 0.83 | −1.27 | −3.27 | −3.36 | 0.98 | −0.96 | −3.47 | −2.11 |
| LOC728688 | XM_001130587 | −3.10 | −3.73 | −4.02 | −3.95 | −3.23 | −3.35 | −3.74 | −2.46 |
| DHCR24 | NM_014762 | −2.67 | −2.89 | −5.38 | −4.77 | −2.49 | −3.34 | −5.48 | −2.86 |

Down regulated and differentially expressed at 18 d

| | | | | | | | | | |
|---|---|---|---|---|---|---|---|---|---|
| HSPB7 | NM_014424 | −1.51 | −4.75 | −4.92 | −5.40 | −0.98 | −4.38 | −5.12 | −8.02 |
| CCBE1 | A_32_P171043 | −2.01 | −2.82 | −3.29 | −4.24 | −1.87 | −3.51 | −3.83 | −7.48 |
| MASP1 | NM_139125 | 0.34 | 0.60 | −0.53 | −3.01 | 0.11 | −0.74 | −1.62 | −6.68 |
| SECTM1 | NM_003004 | −0.56 | −2.06 | −2.46 | −3.05 | −0.39 | −2.42 | −3.21 | −6.42 |
| FAM107A | NM_007177 | −1.82 | −2.35 | −3.13 | −3.69 | −1.60 | −2.91 | −3.60 | −5.66 |
| ADAMTS1 | NM_006988 | −1.51 | −1.16 | −2.57 | −3.45 | −0.96 | −1.42 | −3.50 | −5.06 |
| AOX1 | NM_001159 | −0.22 | 1.02 | 0.08 | −1.08 | −0.34 | 0.02 | −1.07 | −4.76 |
| NTN4 | NM_021229 | −0.87 | −1.78 | −2.22 | −3.57 | −0.80 | −2.33 | −2.66 | −4.74 |
| KLF6 | ENST00000380960 | −0.15 | −0.89 | 0.21 | −3.18 | −0.20 | −0.02 | 0.70 | −4.71 |
| PPL | NM_002705 | 0.28 | −0.27 | −0.54 | −1.55 | −0.28 | −0.75 | −0.91 | −4.68 |
| GPR4 | NM_005282 | 0.13 | −0.41 | −1.58 | −2.50 | 0.39 | −0.44 | −1.84 | −4.39 |
| PTGER2 | NM_000956 | −0.82 | 0.13 | −0.99 | −2.68 | −0.83 | 0.00 | −1.20 | −4.35 |
| CDH13 | NM_001257 | 0.30 | −0.80 | −1.20 | −2.63 | 0.25 | −1.17 | −1.14 | −4.32 |
| GFRA1 | NM_005264 | 0.14 | −0.43 | −2.07 | −1.83 | 0.11 | −1.23 | −3.18 | −4.31 |
| EFEMP1 | NM_004105 | −0.11 | −1.93 | −2.29 | −1.91 | −0.06 | −2.99 | −3.04 | −4.30 |
| C1GALT1 | NM_020156 | −0.38 | −0.88 | −2.00 | −2.53 | −0.41 | −1.34 | −2.28 | −4.26 |
| MGST1 | NM_145791 | 0.32 | 0.28 | −0.94 | −1.92 | 0.02 | −0.01 | −1.04 | −4.22 |
| SQRDL | NM_021199 | −0.36 | −0.81 | −2.05 | −2.08 | −0.39 | −1.02 | −2.31 | −4.06 |
| HSD11B1 | NM_181755 | 0.95 | 2.70 | 0.07 | −1.90 | 0.89 | 1.53 | 0.02 | −3.99 |
| IFI30 | NM_006332 | −0.31 | −0.18 | −1.39 | −2.35 | −0.30 | −0.40 | −2.27 | −3.98 |
| PPP1R14C | NM_030949 | 1.29 | −0.26 | −0.96 | −1.01 | 1.13 | −0.34 | −1.64 | −3.95 |
| RPS6KA2 | NM_021135 | −0.88 | −2.06 | −1.92 | −2.34 | −0.94 | −2.31 | −2.48 | −3.95 |
| ANGPTL5 | NM_178127 | 0.30 | 0.36 | −0.29 | −1.85 | −0.05 | −0.86 | −1.29 | −3.80 |
| CD68 | NM_001251 | 0.80 | 0.96 | −0.62 | −2.44 | 0.58 | 0.47 | −1.14 | −3.79 |
| FGL2 | NM_006682 | −0.16 | 0.25 | −0.98 | −1.35 | −0.13 | −0.04 | −1.76 | −3.76 |
| MRGPRF | NM_145015 | −0.15 | −0.65 | −1.69 | −2.41 | −0.48 | −1.21 | −1.91 | −3.67 |
| PRUNE2 | NM_015225 | −0.09 | −0.09 | −0.61 | −1.73 | −0.50 | −0.94 | −1.46 | −3.62 |
| NT5E | NM_002526 | −0.65 | 0.48 | −1.02 | −2.38 | −0.32 | 0.27 | −1.37 | −3.62 |
| SFRP1 | NM_003012 | −0.01 | 0.35 | −0.54 | −0.44 | 0.27 | 0.10 | −0.64 | −3.56 |
| TNXB | NM_019105 | −0.09 | −1.11 | −1.43 | −2.00 | −0.01 | −1.21 | −0.81 | −3.56 |
| AHNAK2 | BC090889 | 0.06 | −1.32 | −0.99 | −1.81 | −0.47 | −1.80 | −1.68 | −3.43 |
| ADAMTSL4 | NM_019032 | 1.04 | 0.93 | −0.66 | −1.00 | 0.92 | 0.30 | −1.00 | −3.43 |
| C10ORF54 | NM_022153 | −0.57 | −0.80 | −1.20 | −1.60 | −0.51 | −1.41 | −1.67 | −3.40 |
| CAPZA2 | NM_006136 | −0.70 | −0.30 | 0.44 | −2.19 | −0.53 | −0.04 | 0.54 | −3.30 |
| ABCC3 | NM_003786 | −0.20 | 1.83 | −0.06 | 0.55 | −0.30 | 1.06 | 0.65 | −2.90 |
| C13ORF33 | NM_032849 | 2.10 | 1.06 | −0.42 | −1.18 | 2.73 | 1.17 | −0.74 | −2.88 |
| ABI3BP | NM_015429 | 0.72 | −0.71 | −1.49 | −1.44 | 0.48 | −1.44 | −1.51 | −2.76 |
| TFPI | NM_006287 | 1.23 | 1.04 | 0.57 | −0.89 | 1.46 | 0.94 | −0.04 | −2.71 |
| CXCL12 | NM_199168 | −0.26 | 0.04 | −0.40 | 0.20 | −0.38 | −1.03 | −0.85 | −2.67 |
| TNFSF13B | NM_006573 | 2.79 | 1.86 | 0.55 | −0.42 | 2.61 | 2.08 | 0.30 | −2.67 |
| ARID5B | NM_032199 | −0.28 | −0.12 | −0.49 | −1.40 | −0.19 | −0.51 | −0.79 | [symbol] |
| PCYOX1 | THC2563387 | 0.02 | 0.43 | −0.12 | −1.32 | −0.31 | 0.01 | −0.76 | −2.39 |
| APOL3 | NM_145641 | 1.95 | 1.96 | 0.27 | −0.41 | 1.71 | 1.50 | −0.15 | −2.35 |
| RRAS2 | NM_012250 | −1.38 | −1.30 | 0.07 | −1.17 | −1.07 | −0.64 | 0.03 | −2.28 |
| CFL2 | NM_021914 | −1.52 | −1.33 | −1.05 | −1.03 | −1.61 | −1.60 | −0.94 | −2.21 |
| HMGAI | NM_145904 | −0.39 | −0.60 | −0.43 | −0.69 | −0.44 | −0.38 | −0.94 | −2.19 |
| PSPH | NM_004577 | −0.69 | −1.31 | −0.21 | −1.05 | −0.47 | −0.76 | 0.25 | −2.13 |
| ABCA6 | NM_080284 | 0.77 | 0.64 | −0.52 | 0.47 | 0.90 | 0.64 | −0.33 | −2.12 |
| DRAM1 | NM_018370 | 0.57 | 0.52 | −0.29 | −0.73 | 0.64 | −0.09 | −0.86 | −2.11 |
| C1ORF21 | NM_030806 | −0.13 | 0.36 | 0.43 | −0.57 | −0.08 | 0.25 | −0.21 | −2.09 |
| CREG1 | NM_003851 | 0.49 | 0.57 | −0.21 | −0.08 | 0.49 | 0.23 | −0.53 | −2.04 |
| LOC100134569 | AK056484 | −0.17 | −1.56 | −0.52 | −0.91 | 0.01 | −1.16 | −0.28 | −2.03 |
| AKR1C1 | NM_001353 | 1.02 | 0.49 | −0.18 | −0.52 | 0.46 | −0.02 | −0.74 | −2.02 |
| IL6ST | ENST00000381298 | 0.00 | −0.05 | −0.10 | −0.83 | −0.08 | −0.72 | −0.67 | −2.02 |
| ACBD7 | NM_004797 | −0.48 | −0.95 | 0.14 | −0.80 | −0.31 | −0.30 | 0.68 | −1.92 |
| USP53 | BC017382 | 0.26 | 0.41 | −0.21 | −0.75 | 0.21 | −0.07 | −0.45 | −1.91 |
| ASB16 | NM_080863 | −0.47 | −1.00 | 0.48 | −0.64 | −0.25 | −0.22 | 1.12 | −1.85 |
| PROS1 | NM_000313 | −0.08 | −0.28 | −0.10 | −0.63 | −0.39 | −0.91 | −0.46 | −1.82 |
| LYNX1 | NM_177457 | 0.21 | 0.40 | 0.42 | −0.55 | −0.01 | −0.01 | −0.35 | −1.78 |
| SOCS5 | NM_144949 | −0.24 | −0.43 | −0.29 | −0.47 | −0.22 | −0.61 | −0.61 | −1.75 |
| AK3L1 | ENST00000327299 | 2.36 | 1.81 | 1.08 | 0.04 | 2.11 | 1.77 | 0.67 | −1.70 |
| KITLG | NM_000899 | −0.52 | −0.30 | 0.42 | −0.53 | −0.76 | −0.30 | 0.42 | −1.68 |
| POLR2J2 | NM_032959 | −0.53 | −1.06 | 0.51 | −0.36 | −0.42 | −0.20 | 0.94 | −1.67 |
| LOC283174 | AK123849 | 1.69 | 1.29 | 1.68 | 1.88 | 1.30 | 1.03 | 1.51 | −1.64 |
| CCL2 | NM_002982 | −0.46 | 1.39 | −0.29 | 0.59 | −0.49 | 0.88 | 0.39 | −1.63 |
| HSPA4L | NM_014278 | 1.45 | 0.45 | 1.28 | −0.28 | 1.68 | −0.32 | −0.56 | −1.62 |
| USP53 | AF085848 | 0.36 | 0.29 | −0.32 | −0.17 | 0.43 | −0.12 | −0.15 | −1.55 |
| HPSE | NM_006665 | 0.30 | 0.40 | 1.14 | −0.01 | 0.30 | 0.60 | 0.98 | −1.51 |
| STEAP3 | NM_182915 | 0.83 | −0.96 | −0.06 | −0.45 | 0.51 | −0.93 | −0.17 | −1.50 |
| CACNA1B | M94173 | −0.09 | −0.61 | 0.23 | −0.31 | −0.13 | −0.04 | 0.82 | −1.48 |
| ZNF2 | NM_021088 | −0.42 | −0.76 | 0.16 | −0.36 | −0.22 | −0.20 | 0.75 | −1.44 |
| CTSS | NM_004079 | 0.55 | 2.04 | −0.17 | −0.35 | 0.42 | 1.70 | −0.35 | −1.40 |
| ZFP36 | NM_003407 | 1.00 | 0.49 | 0.05 | −0.27 | 1.25 | 0.66 | −0.12 | −1.28 |
| GBP6 | NM_198460 | −0.43 | −0.59 | 0.53 | −0.25 | −0.30 | 0.01 | 0.80 | −1.25 |
| CSNK1A1L | NM_145203 | 0.48 | 0.94 | 1.90 | −0.19 | 0.46 | 1.80 | 2.50 | −1.23 |
| THC2590522 | THC2590522 | −0.20 | 0.40 | 0.06 | −0.07 | 0.05 | 0.51 | −0.15 | −1.22 |
| DHRS3 | NM_004753 | 3.14 | 1.40 | 0.86 | 1.33 | 3.12 | 1.62 | 0.62 | −1.13 |
| LHX1 | NM_005568 | −0.07 | 0.51 | 1.91 | 0.05 | 0.04 | 1.24 | 2.53 | −1.08 |
| CFD | NM_001928 | 1.49 | 3.23 | 2.85 | 3.65 | 1.04 | 2.38 | 1.88 | −1.00 |
| CDK1 | NM_001786 | 0.26 | −0.30 | −0.57 | −2.73 | 0.35 | 0.09 | 0.27 | −0.98 |
| CBX7 | NM_175709 | 1.16 | 0.76 | 1.05 | 0.35 | 1.06 | 0.97 | 0.67 | −0.96 |
| SERPING1 | NM_000062 | 1.34 | 1.62 | 1.78 | 0.36 | 1.02 | 1.20 | 1.05 | −0.91 |
| RAB6A | NM_002869 | 0.02 | 0.82 | 1.64 | 1.52 | 0.29 | 1.51 | 2.09 | −0.87 |
| CD302 | NM_014880 | 0.26 | 0.73 | 0.86 | 0.34 | 0.17 | 0.44 | 1.18 | −0.79 |
| CCNA2 | NM_001237 | −0.06 | −0.27 | 0.08 | −2.05 | −0.08 | 0.95 | 0.81 | −0.77 |
| DPP4 | NM_001935 | 0.31 | −0.25 | 0.85 | 0.56 | 0.26 | −0.24 | 0.61 | −0.71 |
| OLFM1 | NM_006334 | −0.25 | 0.23 | 0.67 | 0.73 | −0.21 | 0.42 | 0.56 | −0.55 |
| PBLD | NM_022129 | −0.12 | −0.88 | 0.47 | 0.67 | −0.15 | −0.63 | 0.95 | −0.51 |
| PDPN | NM_198389 | 1.54 | 1.46 | 1.06 | 0.94 | 1.36 | 0.55 | 0.91 | −0.51 |
| HRH3 | NM_007232 | −0.66 | −0.52 | 1.55 | 0.64 | −0.52 | 0.02 | 1.58 | −0.49 |
| PKP3 | NM_007183 | 1.38 | 1.63 | 1.94 | 0.97 | 0.38 | 2.12 | 2.42 | −0.47 |
| FEM1B | NM_015322 | 0.12 | 0.44 | 1.25 | 0.63 | 0.53 | 0.76 | 1.38 | −0.38 |
| TLR3 | NM_003265 | 1.41 | 1.32 | 1.48 | 1.05 | 0.65 | 1.24 | 1.43 | −0.34 |
| DDHD1 | NM_030637 | −1.02 | 0.01 | 0.99 | 0.81 | −0.87 | 0.69 | 0.91 | −0.30 |
| ACCN1 | NM_183377 | −0.52 | 0.36 | 1.97 | 1.73 | −0.54 | 1.56 | 2.61 | −0.25 |
| SLC11A1 | NM_000578 | −0.56 | 1.17 | 1.93 | 1.32 | −0.55 | 1.24 | 1.74 | −0.19 |
| EMX2 | NM_004098 | 0.08 | 0.25 | 0.96 | 1.08 | 0.12 | 0.60 | 1.10 | −0.19 |
| CD79A | NM_001783 | 0.30 | 0.00 | 1.95 | 0.85 | −0.22 | 0.36 | 1.89 | −0.16 |
| CDS2 | NM_003818 | 0.34 | 0.19 | 0.94 | 1.19 | 0.39 | 0.85 | 1.11 | 0.06 |
| PHOSPHO2 | NM_001008489 | −0.98 | 0.97 | 1.65 | 1.10 | −1.30 | 1.55 | 2.21 | 0.08 |
| GRK5 | NM_005308 | −0.33 | 0.17 | 1.02 | 1.28 | 0.14 | 0.86 | 1.26 | 0.10 |
| SCARF1 | NM_145351 | −0.41 | 0.31 | 1.24 | 1.26 | −0.05 | 0.50 | 1.46 | 0.14 |
| FAM120A | BC007879 | −0.55 | 0.62 | 1.56 | 1.31 | −0.65 | 1.59 | 1.60 | 0.16 |
| GPR77 | NM_018485 | 0.20 | 0.81 | 2.22 | 1.38 | 0.19 | 1.45 | 2.13 | 0.18 |
| VAMP4 | NM_003762 | 0.83 | 1.09 | 1.83 | 1.20 | 0.92 | 1.43 | 2.16 | 0.20 |
| PMP22 | NM_000304 | 0.36 | 0.75 | 1.36 | 1.93 | 0.30 | 0.99 | 1.69 | 0.20 |
| N4BP2L1 | NM_052818 | 2.83 | 2.84 | 2.03 | 1.97 | 2.81 | 2.76 | 1.82 | 0.20 |
| POU3F1 | NM_002699 | −0.50 | 0.65 | 1.69 | 1.80 | −0.33 | 1.29 | 1.95 | 0.21 |
| FAM70B | NM_182614 | 0.22 | 0.14 | 1.11 | 1.32 | 0.17 | 0.56 | 1.52 | 0.22 |
| ANGPTL4 | NM_139314 | 5.36 | 3.69 | 2.86 | 2.96 | 5.58 | 4.66 | 3.07 | 0.23 |
| WFDC3 | NM_080614 | 1.65 | 0.67 | 1.28 | 1.51 | 1.44 | 1.25 | 1.86 | 0.24 |
| FAM8A1 | NM_016255 | −0.13 | 1.04 | 1.87 | 1.45 | 0.15 | 1.73 | 2.41 | 0.28 |
| EDNRB | NM_003991 | 1.05 | 3.22 | 2.98 | 2.63 | 1.92 | 4.19 | 3.38 | 0.29 |
| CASZ1 | NM_017766 | −0.48 | 1.02 | 2.06 | 1.73 | −0.54 | 1.93 | 2.55 | 0.33 |
| TCL1A | NM_021966 | −0.11 | −0.20 | 1.79 | 1.48 | −0.26 | 0.51 | 2.04 | 0.33 |
| DOCK2 | NM_004946 | −1.41 | 1.52 | 2.02 | 2.29 | −1.18 | 2.26 | 2.66 | 0.37 |
| BMPR2 | NM_001204 | 0.02 | 0.64 | 1.45 | −0.69 | −0.15 | 1.07 | 2.03 | 0.41 |
| RAB33A | NM_004794 | 5.14 | 2.05 | 1.82 | 1.95 | 5.11 | 2.60 | 2.71 | 0.45 |
| TRERF1 | ENST00000372922 | −0.27 | 0.66 | 2.65 | 2.20 | −0.36 | 1.56 | 2.91 | 0.51 |
| TFF1 | NM_003225 | −0.15 | 0.44 | 2.22 | 1.79 | −0.21 | 0.67 | 1.95 | 0.56 |
| C13ORF31 | NM_153218 | 0.05 | 1.65 | 1.76 | 1.84 | −0.35 | 1.44 | 1.92 | 0.56 |
| PTGIR | NM_000960 | −0.34 | 1.54 | 2.92 | 1.99 | −0.37 | 2.07 | 2.90 | 0.58 |
| MADCAM1 | NM_130760 | −0.37 | 1.44 | 2.45 | 1.84 | −0.14 | 1.68 | 2.98 | 0.64 |
| DDIT3 | NM_004083 | 4.85 | 1.57 | 2.11 | 1.92 | 4.44 | 1.93 | 2.00 | 0.67 |
| GPR85 | NM_018970 | −0.95 | 2.62 | 2.90 | 3.04 | −1.24 | 2.84 | 2.96 | 0.68 |
| C9ORF98 | NM_152572 | 0.72 | 0.56 | 2.65 | 2.30 | 0.35 | 1.31 | 2.33 | 0.71 |
| RUNDC2B | AK023827 | 0.20 | 0.76 | 2.04 | 2.05 | 0.09 | 1.16 | 2.27 | 0.72 |
| CREB5 | NM_182898 | 1.14 | 1.17 | 2.21 | 2.01 | 1.05 | 1.48 | 2.52 | 0.75 |
| CFB | NM_001710 | 1.78 | 3.83 | 2.99 | 2.34 | 1.25 | 3.24 | 2.80 | 0.78 |
| TINAGL1 | NM_022164 | −0.17 | −0.03 | 1.87 | 2.21 | −0.21 | 1.21 | 2.30 | 0.81 |
| RUFY3 | NM_014961 | 0.98 | 0.82 | 1.59 | 1.95 | 0.76 | 1.63 | 2.15 | 0.86 |
| FLJ30901 | AK055463 | −0.35 | 0.39 | 2.37 | 2.11 | −0.26 | 1.40 | 2.77 | 0.88 |
| BPTF | NM_182641 | 0.07 | 1.08 | 2.06 | 2.06 | −0.34 | 1.55 | 2.49 | 0.93 |
| LOC100133050 | XM_001126539 | 0.15 | 1.30 | 2.72 | 2.12 | 0.01 | 1.41 | 2.50 | 1.03 |
| MCL1 | NM_021960 | −0.52 | 2.07 | 2.51 | 2.21 | 0.29 | 2.52 | 2.71 | 1.06 |
| SPDYE3 | NM_001004351 | −0.29 | 0.32 | 2.16 | 2.12 | 0.05 | 1.53 | 2.60 | 1.09 |
| IGSF6 | NM_005849 | −0.01 | 4.00 | 4.29 | 2.66 | −0.01 | 3.26 | 3.84 | 1.09 |
| PRAMI | NM_032152 | 0.17 | 1.28 | 2.60 | 2.49 | −0.04 | 1.60 | 2.46 | 1.16 |
| CENPL | NM_033319 | 0.20 | 0.77 | 1.62 | 2.23 | 0.27 | 1.58 | 2.31 | 1.17 |
| GPR144 | ENST00000334810 | −0.56 | 0.55 | 1.99 | 2.39 | −0.37 | 1.63 | 2.45 | 1.17 |
| OXER1 | NM_148962 | −0.20 | 0.60 | 2.15 | 2.40 | −0.14 | 1.28 | 2.67 | 1.17 |
| BCAS4 | BC056883 | −0.10 | 0.74 | 1.80 | 2.27 | 0.16 | 1.39 | 2.24 | 1.20 |
| TMEFF2 | AB004064 | 0.41 | 1.16 | 2.80 | 2.88 | 0.77 | 1.87 | 2.71 | 1.20 |
| EIF4B | NM_001417 | 0.23 | 0.77 | 5.25 | 3.16 | 0.11 | 2.65 | 5.52 | 1.24 |
| CTRL | NM_001907 | −0.32 | 0.67 | 2.43 | 2.28 | −0.43 | 1.33 | 2.72 | 1.26 |
| DAB1 | NM_021080 | −0.31 | 1.97 | 2.89 | 3.46 | −0.33 | 3.16 | 4.03 | 1.33 |
| TMEM105 | NM_178520 | 0.77 | 3.49 | 3.57 | 2.81 | 1.37 | 4.22 | 3.78 | 1.35 |
| FAM63A | NM_018379 | 0.60 | 1.37 | 2.87 | 2.48 | −0.25 | 2.04 | 3.15 | 1.36 |
| RPS6KA3 | NM_004586 | 0.25 | 1.69 | 2.37 | 2.49 | 0.35 | 2.31 | 2.51 | [symbol] |
| HIPK2 | BC041926 | −0.47 | 0.36 | 2.53 | 2.77 | −0.67 | 1.55 | 2.57 | 1.38 |
| FAM179A | THC2697920 | −0.08 | 1.58 | 3.05 | 2.55 | −0.03 | 2.24 | 3.06 | 1.41 |
| SMAGP | NM_001031628 | −1.53 | 1.97 | 3.30 | 3.10 | −1.51 | 2.05 | 3.16 | 1.46 |
| MAG | NM_080600 | −0.25 | 0.59 | 2.22 | 2.78 | −0.09 | 1.32 | 2.90 | 1.47 |
| C9ORF50 | NM_199350 | 0.29 | 2.61 | 3.13 | 2.62 | 0.40 | 3.07 | 3.53 | 1.61 |
| ALCAM | NM_001627 | −1.43 | 2.33 | 3.41 | 3.47 | −1.07 | 2.51 | 3.06 | 1.69 |
| PDK4 | NM_002612 | 1.90 | 4.53 | 4.02 | 4.48 | 2.59 | 5.07 | 3.35 | 1.74 |
| LOC729137 | AK090442 | −0.24 | 2.32 | 4.47 | 3.62 | −0.17 | 2.94 | 3.88 | 1.80 |
| HBD | NM_000519 | −0.22 | 1.27 | 2.97 | 5.40 | −0.54 | 3.47 | 6.28 | 1.82 |
| HIP1 | NM_005338 | −0.70 | 1.96 | 3.45 | 3.19 | −0.44 | 2.49 | 3.75 | 1.83 |
| QKI | NM_206855 | −0.78 | 2.09 | 3.32 | 2.94 | −0.37 | 2.82 | 3.70 | 1.88 |
| 1KZF2 | NM_001079526 | 1.98 | 1.46 | 2.57 | 3.28 | 1.90 | 2.09 | 3.09 | 1.92 |
| LOC100129115 | AK095213 | 0.14 | 1.97 | 3.59 | 3.02 | 0.16 | 2.79 | 3.77 | 1.95 |
| TEC | NM_003215 | 2.01 | 2.59 | 4.25 | 3.10 | 1.60 | 3.07 | 4.28 | 1.96 |
| ZEB2 | NM_014795 | 0.38 | 2.27 | 2.63 | 3.02 | 0.57 | 2.76 | 3.01 | 1.98 |
| TTC39C | NM_153211 | −0.20 | 1.89 | 3.01 | 3.16 | −0.20 | 2.54 | 3.43 | 2.00 |
| FAM131B | NM_014690 | −0.67 | 1.51 | 3.38 | 3.87 | −0.30 | 2.53 | 3.85 | 2.01 |
| ZNF238 | NM_006352 | 0.42 | 3.72 | 3.89 | 3.54 | −0.09 | 4.22 | 4.47 | 2.03 |
| ABCA1 | NM_005502 | 4.62 | 3.80 | 3.69 | 3.55 | 4.48 | 3.29 | 3.31 | 2.07 |
| LRRN2 | NM_201630 | −0.62 | 0.80 | 3.52 | 3.62 | −0.68 | 2.23 | 3.52 | 2.07 |
| GNG2 | NM_053064 | 0.53 | 2.95 | 3.75 | 3.56 | 0.82 | 3.52 | 3.97 | 2.12 |
| TMEM33 | BU567141 | −0.83 | 0.70 | 5.78 | 4.27 | −0.65 | 2.72 | 5.82 | 2.25 |
| UNKL | ENST00000074056 | −0.18 | 3.39 | 4.86 | 4.31 | −0.13 | 3.81 | 4.94 | 2.40 |
| CD14 | NM_000591 | −0.19 | 4.45 | 3.93 | 3.84 | −0.10 | 4.66 | 4.28 | 2.47 |
| TM6SF1 | NM_023003 | −0.06 | 3.67 | 4.72 | 4.22 | −0.03 | 4.02 | 4.72 | 2.59 |
| SLC15A3 | NM_016582 | 1.68 | 3.99 | 4.27 | 4.52 | 1.35 | 4.09 | 4.22 | 2.73 |
| EPHB6 | NM_004445 | −0.44 | 2.63 | 4.42 | 4.28 | −0.19 | 3.21 | 4.68 | 2.81 |
| NCF1 | NM_000265 | 1.26 | 4.43 | 4.79 | 4.36 | 0.65 | 4.63 | 4.77 | 3.04 |
| CEACAM1 | NM_001712 | 1.40 | 3.29 | 4.97 | 4.53 | 1.16 | 4.30 | 5.10 | 3.13 |
| PVT1 | NR_003367 | 0.08 | 2.69 | 4.13 | 4.35 | 0.31 | 3.81 | 4.75 | 3.20 |
| NCF4 | NM_000631 | 0.07 | 5.93 | 6.82 | 4.71 | 0.04 | 6.08 | 6.56 | 3.22 |
| LMO2 | NM_005574 | 1.83 | 3.28 | 4.54 | 4.62 | 1.30 | 3.74 | 4.55 | 3.28 |
| CPA3 | NM_001870 | −0.72 | 3.43 | 7.30 | 5.00 | −0.64 | 5.89 | 7.75 | 3.30 |
| SNX12 | NM_013346 | −0.03 | 3.33 | 4.22 | 4.45 | −0.13 | 3.90 | 4.46 | 3.37 |
| TREM2 | NM_018965 | −0.41 | 3.34 | 5.10 | 5.45 | −0.42 | 3.36 | 4.87 | 3.52 |
| GDA | NM_004293 | 0.02 | 6.36 | 5.66 | 5.11 | 0.01 | 7.00 | 6.19 | 3.56 |
| RAD51L1 | NM_133510 | 0.79 | 2.22 | 6.99 | 5.82 | 0.53 | 4.53 | 7.19 | 4.00 |
| TLR6 | NM_006068 | 0.28 | 5.87 | 5.91 | 5.61 | −0.30 | 6.03 | 5.76 | 4.17 |
| THC2586092 | THC2586092 | −0.63 | 5.87 | 5.64 | 5.27 | −0.43 | 5.91 | 5.72 | 4.23 |
| CD37 | NM_001774 | 0.01 | 5.18 | 6.29 | 5.72 | −0.08 | 5.88 | 6.15 | 4.27 |
| MAN1A1 | NM_005907 | −0.29 | 4.21 | 5.63 | 5.54 | −0.10 | 5.22 | 5.94 | 4.34 |
| TNFSF13 | NM_172088 | 0.04 | 4.23 | 5.74 | 5.96 | −0.16 | 4.75 | 5.70 | 4.47 |
| DOK2 | NM_003974 | 0.05 | 7.25 | 7.62 | 6.67 | −0.11 | 7.61 | 7.31 | 4.50 |
| SFXN5 | NM_144579 | −0.39 | 4.89 | 5.89 | 5.58 | −0.64 | 5.67 | 6.28 | 4.55 |
| HK3 | NM_002115 | −0.76 | 5.77 | 6.65 | 6.75 | −0.06 | 5.79 | 6.52 | 4.60 |
| C15ORF48 | NM_032413 | 4.30 | 5.15 | 6.46 | 5.97 | 4.89 | 5.90 | 6.51 | 4.63 |
| ITGAX | NM_000887 | 0.36 | 5.02 | 7.53 | 7.07 | 0.31 | 4.12 | 7.46 | 5.15 |
| FBXO24 | NM_033506 | −0.11 | 6.65 | 7.96 | 6.62 | −0.03 | 7.35 | 7.95 | 5.45 |
| CORO1A | NM_007074 | −1.01 | 7.28 | 7.18 | 6.63 | −1.31 | 7.43 | 7.18 | 5.55 |
| ZNF608 | NM_020747 | 0.32 | 6.85 | 7.50 | 7.26 | 0.26 | 6.99 | 7.81 | 5.58 |
| RASSF4 | NM_032023 | 1.85 | 6.40 | 6.85 | 7.28 | 1.40 | 7.02 | 6.71 | 5.63 |
| CFP | NM_002621 | −0.26 | 7.99 | 8.21 | 7.61 | −0.23 | 8.44 | 7.98 | 5.76 |
| VAV3 | NM_006113 | −0.18 | 6.80 | 7.56 | 7.32 | 0.08 | 7.21 | 7.58 | 5.92 |
| CD36 | NM_001001547 | 0.71 | 7.89 | 10.04 | 8.88 | 0.38 | 8.45 | 9.43 | 6.75 |
| ALOX5AP | NM_001629 | 0.03 | 10.00 | 10.43 | 8.72 | −0.01 | 10.45 | 10.20 | 7.01 |

Gene topology of the GOI list was visualized with Gene Expression Dynamics Inspector (GEDI) (8). The parameter settings to generate Self Organizing Maps (SOMs) are shown in Table 4. Gene ontology was performed with DAVID (Database for Annotation, Visualization and Integrated Discovery, http://david.abcc.ncifcrf.gov/). Gene sets from each time point were loaded and analyzed to discover the main biological processes at each time point. The stringency for functional clustering was set on “high” (9).

TABLE 4
Overview of parameter settings to generate the SOMs in GEDI.

| Grid size SOM | X = 11 | Y = 12 |
|---|---|---|
| SOM training quality | Maximum | |
| | First Phase | Second Phase |
| Training iterations | 80 | 160 |
| neighborhood radius | 4.0 | 1.0 |
| Learning Factor | 0.6 | 0.1 |
| Conscience | 5.0 | 5.0 |
| Neighborhood Block Size | 2.0 | 1.0 |

| | |
|---|---|
| Random Seed | 1 |
| Initialization method | Linear Initialization |
| Distance metrics | Euclidean Distance |

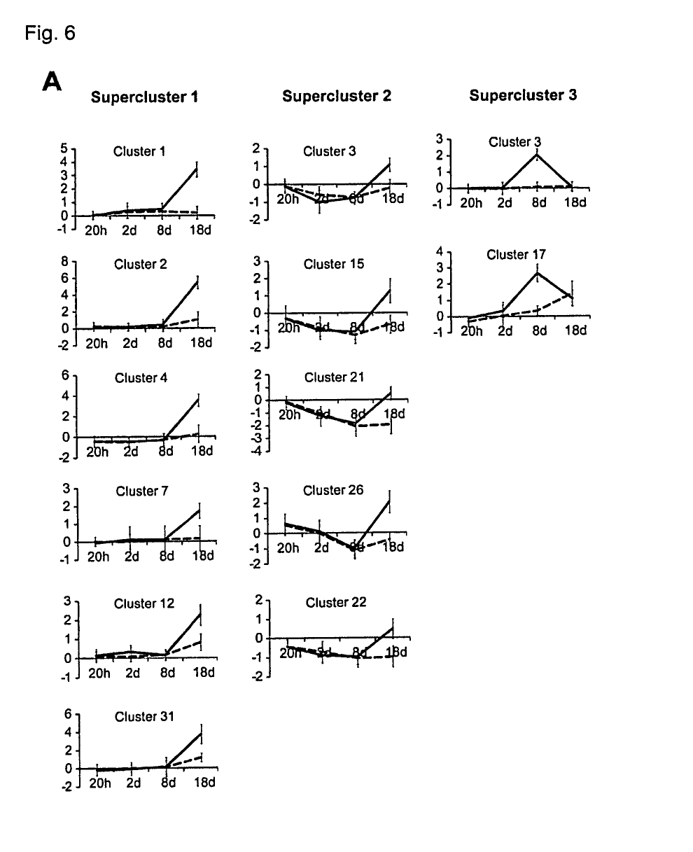

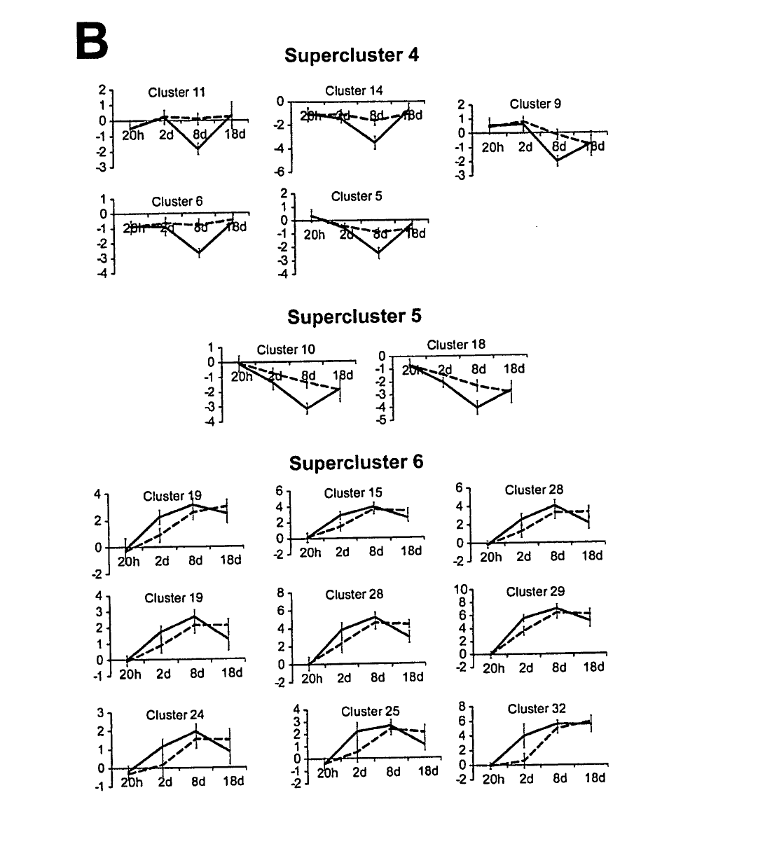

Co-expressed genes were clustered according to their temporal profile in the decalcified and non decalcified Collagraft™ structures utilizing the SOM algorithm of GEDI with the “reducing neighborhood block” parameter set to 1 in the first training phase (10). 110 Clusters with an average gene size of 11 (t6) genes per cluster were obtained. For each cluster, the average gene expression and standard deviation for every time point was calculated and statistically compared between decalcified versus non decalficied Collagraft™. Clusters having no significant differences at any time point were omitted from further analysis (student t-test, p-value cut-off p<0.001). The remaining 64 clusters were ranked according to their p-value starting with the lowest p-value first. The first 32 clusters (representing 553 genes or 58% of the GOI list) were used for subsequent analysis. Temporal profiles of the metagenes (=average expression of the genes within a cluster) was plotted for each of the 32 clusters which could be organized in 6 superclusters (FIG. 6). Genes from each supercluster were loaded in Ingenuity Pathway Analysis (Ingenuity Systems, Redwood City, Calif.) for gene network reconstruction. Gene networks were built with a restriction of 70 genes per network and 25 networks per supercluster (Table 3).

Quantitative PCR. Complementary DNA (cDNA) was obtained by reverse transcription of 1 pg of total RNA with Oligo (dT)20 as primer (Superscript Ill; Invitrogen, Merelbeke, Belgium). Sybr Green PCR was performed in 10 pl reaction in a Rotor-Gene-Q (Qiagen) with following protocol: 95° C. for 3 seconds, 60° C. for 20 seconds. Primer sequences for specific Sybr green PCR was performed with human specific primers (Table 5). Taqman PCR primer/probe combinations (Applied Biosystems) were used in the in vitro osteogenesis assays.

TABLE 5
Primer sequences designed to detect human specific transcripts
for several target genes with Sybr green PCR.

| | | | SEQ |
|---|---|---|---|
| Gene | Sense | Anti Sense | ID No |
| Osterix | AGTGACCTTTCAGCCTCCAA | GGGAAAAGGGAGGGTAATCA | 1/2 |
| Bone Sialo | CCGAAGAAAATGGAGATGACA | CCTCTCCATAGCCCAGTGTT | 3/4 |
| Protein | | | |
| Osteocalcin | GTGCAGCCTTTGTGTCCAA | GCTCACACACCTCCCTCCT | 5/6 |
| ANO1 | CCGGAGCACGATTGTCTATG | CTCGACGTTTTCACCGTTGT | 7/8 |
| NDK2 | TCAACATTGACGCACTCCAG | GAGGCATCCACGACCTCATA | 9/10 |
| OPN | TAAATTCTGGGAGGGCTTGG | GATGCCTAGGAGGCAAAAGC | 11/12 |
| SLN | GATCCTCTTCAGGAGGTGAGG | ACAGCTCCCGGGTGTTTATC | 13/14 |
| TNSF11 | CCTTTCAAGGAGCTGTGCAA | TGGGAACCAGATGGGATGT | 15/16 |

Statistical Analysis. Experiments were carried out in triplicate. The error bars represent the standard error of the mean when cells from multiple donors are used. Standard deviations are shown when experiments are performed with a hPDC cell pool (n=3). Statistical comparison between experimental conditions was performed with a Mann-Whitney U test. A p-value ≦0.05 was considered as being statistically significant.

### Example 2: Results

To study the role of CaP in ectopic bone formation by MSCs, we developed a model system in which synthetic CaP carrier structures (Collagraft™) were decalcified, leaving a collagen matrix behind, prior to cell seeding and implantation. In these structures, ectopic bone formation by hPDCs was absent (6). Since the process of ectopic bone formation by hPDCs in a Collagraft™ carrier fully develops without adding additional growth factors, we hypothesized that CaP may initialize osteogenic gene networks shortly after implantation. To address this hypothesis, we set out to examine genome wide gene expression of hPDCs engrafted on decalcified and non-decalcified Collagraft™ carriers before and after subcutaneous implantation in nude mice. Utilizing bioinformatics, we inferred gene networks and signaling pathways based on differential gene expression over time and between the two conditions. Subsequently, differential gene expression and activation of several signaling pathways was validated with quantitative PCR and western blot analysis. Finally, we tested if activation of the identified in vivo pathways could promote osteogenic differentiation of hPDCs in vitro and in vivo.

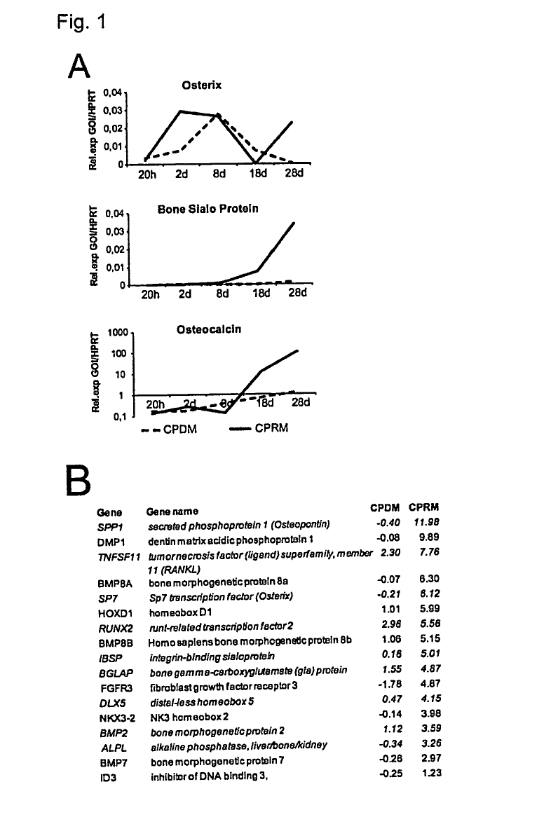

Osteogenic gene signature establishes within three weeks after implantation. To determine the time window when osteogenic differentiation occurs in vivo, hPDCs were seeded on calcium phosphate depleted matrices (CPDM) and non decalcified, calcium phosphate rich (CPRM), Collagraft™ carriers and subcutaneously implanted for 2, 8, 18 and 28 days. As shown in FIG. 1, the early bone marker Osterix (OSX) and Bone Sialo Protein (BSP) and Osteocalcin (OC), two markers reflecting osteoblast maturation, were upregulated in the CPRM within 18 days. Based on the expression of these three markers, we considered 20h after seeding, 2 days, 8 days and 18 days as four time points to explore gene expression with microarray. Indeed, the genes of interest (GOI) returned several early and late osteoblast markers that were highly expressed in CPRM (FIG. 1B and Table 2) indicating that the time points were well chosen. Interestingly, the osteocyte marker DMP1 but not Sclerostin or PHEX was upregulated (FIG. 1B). These results indicate that osteogenic differentiation from progenitor cell to mature osteoblast occurred within the first three weeks of implantation.

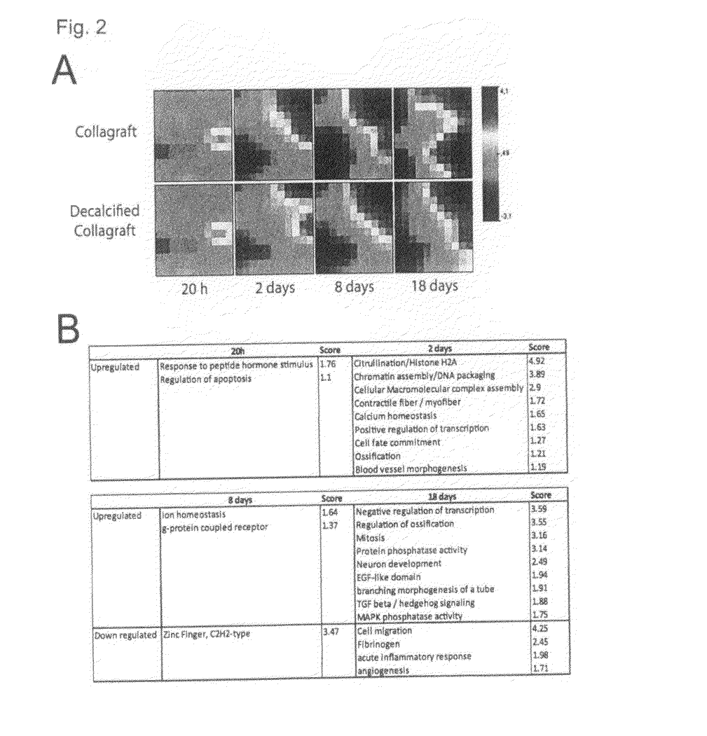

Calcium phosphate modulates osteogenic gene network dynamics in vivo. Due to the nature of the microarray data (time series in two independent conditions), we opted to arrange the GOI in Self Organizing Maps (SOMs). SOMs assign genes with a comparable expression over time to the same tile in a 2D plot. Hence, genes plotted in the close vicinity of each other on the SOM behave very similar throughout the experiment, whereas genes assigned to tiles further away from each other behave differently. As each tile is color coded according to the average gene expression (light gray=low expression, black=high expression), gene topologies can be visualized into distinct patterns (10). As shown in FIG. 2A, gene topologies were comparable between CPRM and CPDM at 20 h and two days after implantation. However, distinct patterns are noted when comparing gene topologies at two days (in vivo) and 20 h after seeding (in vitro). Interestingly, the SOMs of CPDM at eight days and 18 days appeared similar to the ones at respectively two and eight days in CPRM. Changing the parametric values of the SOMs generated different visual patterns, but did not affect the interpretation of the results. The observation that gene topologies in both types of matrices display very similar changes when transferred from an in vitro to in vivo environment suggest that implantation of cell seeded scaffolds in a subcutaneous pocket (“wound” environment) is sufficient to ‘activate’ the hPDCs. Upon implantation, gene topology dynamics progressed faster in CPRM as compared to CPDM suggesting that CaP may promote or ‘accelerate’ the osteogenic program of hPDCs.

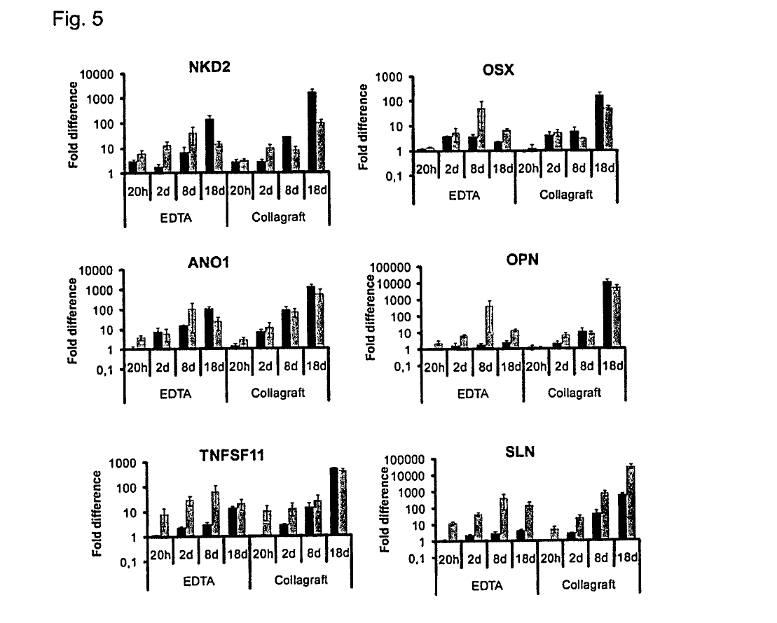

Because gene topology is a meta analysis based on the expression of a priori defined genes of interest, validation of single gene expression with qPCR is appropriate. Here, the expression of six differentially expressed genes in the array was validated with qPCR using human specific primers. Two genes, Osterix (OSX) and Osteopontin (OPN) are well established bone markers. Based on the microarray data, the other four genes, Anoctamin-1 (ANO1), Naked Cuticle 2 (NKD2), Sarcolipin (SLN) and Tumor Necrosis Factor (Ligand) Superfamily member 11 (TNFSF11 also known as RANKL) were upregulated and differentially expressed between CPRM and CPDM at 8 and 18 days. Hence, these genes can be considered as putative early bone markers for in vivo bone formation (FIG. 5).

Because microarrays are not designed to detect species specific transcripts, the measured gene expression reflects cellular processes from both engrafted and host cells. Gene ontology (GO) analysis identified these cellular processes related to “cell survival” at twenty hours after seeding, “chromatin remodeling” and “positive regulation of transcription” at two days after implantation and “mitosis”, “osteogenesis”, “sprouting” (tube morphogenesis) and “neuron development” at 18 days after implantation (FIG. 2B). Interestingly, at two days post implantation the dataset was little enriched for genes associated with “osteogenesis” and “blood vessel morphogenesis” suggesting that the decision making point for osteogenic differentiation might occur early on after implantation. In addition, the transient expression of genes associated with “fiber contractility” (associated with cell migration), “inflammation”, “gene transcription”, and “angiogenesis” between 2 and 18 days further underscores a significant role for the host cells in this process.

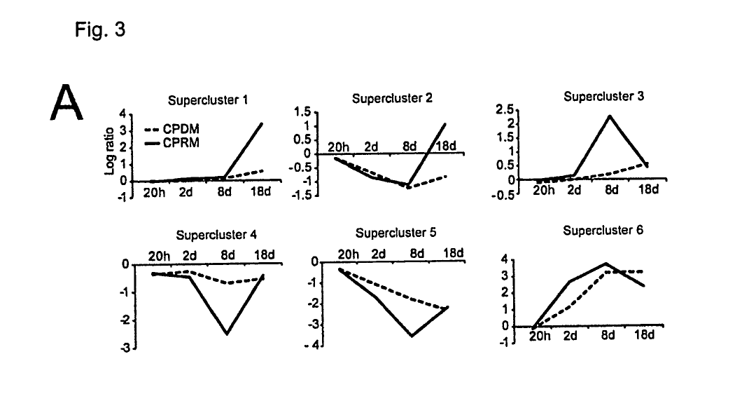

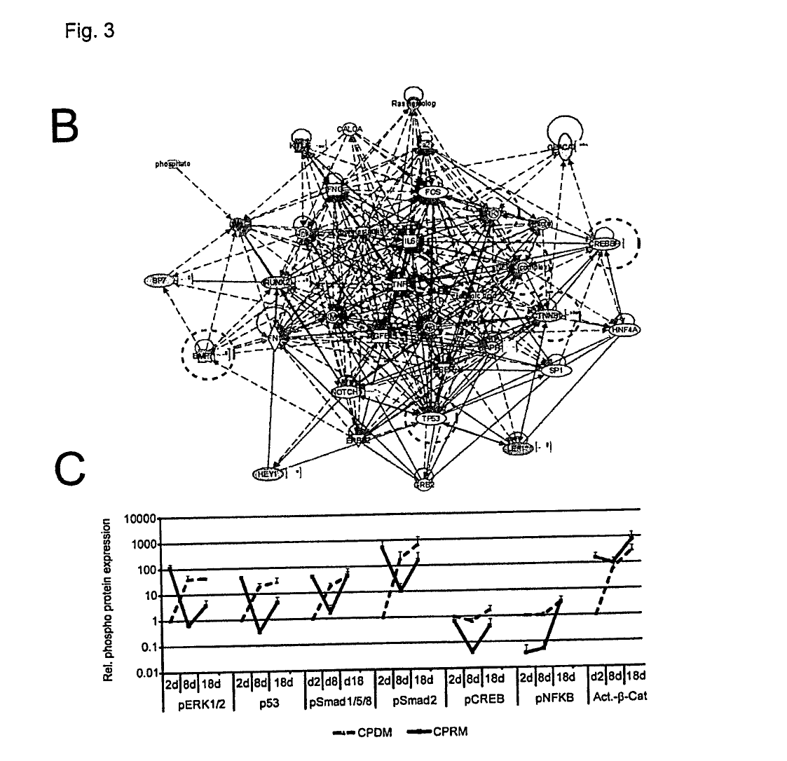

Mapping the hub gene network. Although GO and SOM analysis described the early biological events during ectopic bone formation, they provided little insight into the molecular signaling pathways that were activated in CPRM. To address this issue, we assumed that co-expressed genes sharing similar temporal profiles are regulated by common hub genes. Therefore, co-expressed genes in both experimental conditions were clustered into six superclusters (FIG. 3A, FIG. 6). Subsequently, the genes of the six superclusters were loaded in Ingenuity Pathway Analysis to build gene networks for each supercluster. Based on the retrieved network maps (Table 3), hub genes were selected and mapped into a gene network connecting the hub genes with “direct” and “indirect” gene/protein interactions (FIG. 3B). As expected, expression of hub genes known to be involved in bone formation such as beta catenin, LEF1, Runx2, OSX, ALP, BMP7 and Notch/Hey1 were up regulated in the CPRM. In contrast, KITLigand was down regulated. Interestingly, several hub genes linked to TGFβ (TGFβ1), MAPK (p38, ERK1/2), TNFα (TNFα, IFNγ, IL6, NFκB), EGF (ERBB2, GRB2, EGFR), and p53 signaling (TP53) were not differentially expressed at the transcriptional level.

TABLE 3
Table 3: Gene networks generated with IPA for each supercluster. Hub genes
that were used to map the hub gene network are annotated in bold.

| Network | Cluster name | Score | Function |
|---|---|---|---|
| | Supercluster 1 | | |
| 1 | ACVRL1, ALPP, amino acids, AOC2 (includes EG:314), ARL2BP, ARPC4, | 53 | Cellular |
| | BARX2, BMP7, CALCA, CELSR2, CHST1, CREBBP, DLG4, DLX5, | | Growth and |
| | DOCK4, DUSP13, EPO, ERBB2, FN1, FOS, FSTL3, GFI1, GRB2, HEY1, | | Proliferation, |
| | HOMER2, HOXA11, HOXD1, HTR2B, ITGA7, ITGA8 (includes EG:8516), | | Cellular |
| | ITGB4, JUB, KCNAB1, keratan sulfate, LIMD1, LRRC1, MAST4, MFAP2, | | Development, |
| | MIR125A (includes EG:406910), MIR24-1 (includes EG:407012), | | Tissue |
| | MIRN140, MKI67, NPNT, NPTX2, OGDHL, PDGF BB, PDGFC, PGAM2, | | Development |
| | PLCH1, PLXNC1, PNOC, PRSS35, RASL10A, RUNX3, SEMA7A, SFN, | | |
| | SGK223, SHROOM1, SLC25A4, SLC27A1, SMAD1, SMAD1/5, SPARC, | | |
| | TAGLN, TGFB1, TGFB1I1, TM7SF4, TPSB2, UBA52, ZCCHC12 | | |
| 2 | 20alpha-hydroxycholesterol, 22(S)-hydroxycholesterol, ACTG2 (includes | 41 | Cell |
| | EG:72), Actin, AGAP2, Akt, Alp, ALPL, ALPP, Ap1, APCDD1, AXIN1, | | Morphology, |
| | BMP, BMP4, BMP7, BMP8A, BMP8B, CALCA, CHRDL2, CHRNA1, | | Skeletal and |
| | COL10A1, creatine, CTNNβ-TCF/LEF, DCT, DIO2, DLX2, DLX5, DMP1, | | Muscular |
| | DNER, DSPP, DUSP2, DVL1, ERK, ERK1/2, ESRRG, FGF23, GABRR1, | | System |
| | GRB10, HEY1, HEY2, HOMER2, IBSP, JAG1, Jnk, KCNQ2, LEF1, | | Development |
| | MEGF10, MSTN, MSX1, MSX2, NKX3-2, NOG, NOTCH2, NOTCH3, | | and Function, |
| | NOTCH4, P38 MAPK, PDGFC, PITX2, PTPRZ1, ROR2, RSU1, RUNX2, | | Tissue |
| | SMOOTH MUSCLE ACTIN, SOST, SP7, Tgf beta, triiodothyronine | | Development |
| | reverse, TSC22D3, WNT10B | | |
| 3 | ABCA4, ALPL, ANO1, APCDD1, APP, ATP, ATP2A2, BCL2, beta- | 39 | Molecular |
| | estradiol, C20ORF160, CDX1, CKM, COL9A2, CRH, CRHR1, CRYBA4 | | Transport, |
| | CRYBB1, CRYBB2, CTSC, cyclic GMP, DKK1, DLX1, DLX5, FABP6 | | Nucleic Acid |
| | FAM78A, GAD2, GZMA, HIVEP3, Hsp70, IGFBP6, IL1B, JUN, KANK4 | | Metabolism, |
| | KIAA1211, LAMB1, LGALS2, LINGO1, MATN2, melatonin, MIR195 | | Small |
| | (includes EG:406971), MIR297-2, MIRLET7B (includes EG:406884) | | Molecule |
| | MIRLET7G (includes EG:406890), MMP7, NCAM1, NFE2L1, NPTX1 | | Biochemistry |
| | NR4A2, PDE9A, PELI2, phosphocreatine, PI3, RCN2, retinoic acid | | |
| | SATB2, SEMA6A, SFRP1, SMPD3, SPARC, sphingomyelinase, TGFB3 | | |
| | TH, TNF, TNFRSF19, TRAF3IP3, TSC22D3, WNT10B, WNT5A, WWOX | | |
| | YWHAZ | | |
| | Supercluster 2 | | |
| 4 | Actin, ADAMTS4, ADAMTS5, Alpha catenin, ALPL, BAIAP2, C21ORF33, | 51 | Connective |
| | Ca2+, CASR, CBFB, CCL5, CNTN1, COL8A1, COMP, CPNE4, DCTN1, | | Tissue |
| | DCTN2, DLG4, DUSP10, DYNC1H1, DYNC1I1, DYNLL2, E2F4, EML4, | | Disorders, |
| | EPS8L1, EXOSC5, FHL1, GRIK1, HNF4A, HOMER2, HOOK3, HPCAL1, | | Developmental |
| | HTT, IPP, KIAA1217, KIF4A, KLHL12, LIN7B, MATN3, MATN4, NCAPD2, | | Disorder, |
| | NDC80, NFKBIL2, NUF2, ONECUT1, PPP5C, PTGDS, PTH1R, PVRL2, | | Genetic |
| | Ras homolog, SEMA7A, SGIP1, SPC24, SPC25, Sphk, STAU1, STX18, | | Disorder |
| | TGFB1, TMTC4, TPSB2, TPST2, TRP, TRPC3, TRPC5, TUBB2C, UTP3, | | |
| | WDR12, WNT4, ZFP36, ZWINT (includes EG:11130) | | |
| 5 | 24,25-dihydroxyvitamin D3, ACTG2 (includes EG:72), ADAM19, | 42 | Cellular |
| | ADAMTS7, Alp, ALPL, ANKRD1, APP, ASCL1, beta-estradiol, BICD1, | | Growth and |
| | BMPR1A, BTBD3, C21ORF33, CD38, CMTM8, CSF2, CTNNβ-TCF/LEF, | | Proliferation, |
| | CYTSB, Delta/Jagged, DLL1, DLL3, DLX4, EGFR, ERG, FER (includes | | Cellular |
| | EG:2241), FSH, GRB2, GYPA, IFNG, IGKV1-117, IL8, IL21R, ITM2C, | | Development, |
| | JAG1, JAG2, LFNG, Lh, MAGI1, MATN2, MB, MCAM, MFAP5, MIR24, | | Embryonic |
| | MIR199A1, MIR34A (includes EG:407040), MSX1, NCSTN, NFkB | | Development |
| | (complex), NOTCH1, NOV, NUMBL, PECAM1, PHTF2, PPP1R14B, | | |
| | PSENEN, PTHLH, RASAL2, RECQL4, SAA, SEPN1 (includes EG:57190), | | |
| | SMARCB1, TNF, TP53I11, TUBB2C, TWIST1, vitamin B12, ZDHHC23, | | |
| | ZEB2 | | |
| | Supercluster 3 | | |
| 6 | ADAM22, ADRBK2, Arf, ARF6, ATP2B2, beta-estradiol, CCL17, CCL22, | 31 | Genetic |
| | CCR4, CHRM1, CHRM3, CNTN2, CNTNAP2, CORT, CXCR4, CYR61, | | Disorder, |
| | CYTH1, CYTH2, CYTH3, DLG2, DYNLT1, ERC2, FRK, FZD4, G-protein | | Neurological |
| | beta, GAB1, GFI1B, GIT1, GNAQ, GRIP2, GRK4, GRK5, HDAC9 (includes | | Disease, |
| | EG:9734), HECW1, HOXB8, IL4, II3r, IL3RA, IPCEF1, isopentenyl | | Psychological |
| | diphosphate, Jnk, KCNA2, KCNA4, LGI1, MPDZ, MYLK, NCAN, NGF, | | Disorders |
| | PBX1, PHC1, PHC2, PLD1, PPFIA1, PPFIA4, PPP1R9B, Ptk, PTK2B, | | |
| | RAC1, RHO, SCNN1A, SIX3, SLAMF7, SSR4, SSTR1, SSTR4, TAGLN, | | |
| | taurolithocholic acid, TBX21, TNF, TUBG1 | | |
| | Supercluster 4 | | |
| 7 | AKIRIN1, APP, ASB8, beta-estradiol, C2ORF49, C3ORF19, C5ORF22, | 53 | Protein |
| | CAT, CDKN2A, CELF1, Creb, DNAJC3, DRAP1, EGFR, EIF2AK3, | | Synthesis, |
| | EIF2B1, EIF2B3, EIF2S1, EIF2S2, ERO1L, ERP44, FAM178A, FRZB, | | Lipid |
| | GABPB2, GDAP2, heme, HNF4A, IL24, KBTBD7, KIF5A, KIF5C, | | Metabolism, |
| | KLHDC3, KLHL13, LSS, MAPKAP1, METAP2, METTL2B, MIR291A | | Small |
| | (includes EG:100049715), MIR301A (includes EG:407027), MIRLET7E | | Molecule |
| | (includes EG:406887), MTORC2, NFYB, NMNAT1, Nos, PCBP2, PJA2, | | Biochemistry |
| | Pkc(s), PLAA, PPARGC1B, PPP1R15A, RPS9, RUNX1T1, SLK, SUPT3H | | |
| | (includes EG:8464), TADA2B, TAF12 (includes EG:6883), TAF9B, TCEB1, | | |
| | TCEB2, TMEM17, TNF, TOPORS, TP53, TP53INP2, TRAK2, TXNL4B, | | |
| | UBR1, UBR2, UBXN7, VPS41 | | |
| | Supercluster 5 | | |
| 8 | ABCD1, AGTBP1, AIF1, ALOX5AP, BLVRB, BMP1, C21orf91, C4BPB, | | |
| | C70RP6, CCL6, CCL8, GDCD85B, CREG1, CYG1, CYP2U1, DACH2, | | |
| | DNMT3A, DYNC1, EPH84, EPX, ERBB2, FRK, FXYD6, GCC2, GLYAT, | | |
| | HNF4A, HUS1, IF127L2, IFNG, IL4, IKZF5, KITLG, LSMD1, LACTB, | | |
| | LTC4S, LY6E, MARCO, MCM3, MID1IP1, MIR214 (includes EG:406996), | | |
| | MREG, NAA3C, NCOR1, NUDT1, ORC5L, PCNA, PPFIA, PPP2CA, | | |
| | PROS, PNP, RB1, retinoic acid, RFC4, RFC5, RMND, SAMD9, SAMSN1, | | |
| | SRGN, ST3GAL4, TGFb1, TNFRSF23, TPM4, TPSB2, TXNLN, USP25, | | |
| | ZBT5 | | |
| | Supercluster 6 | | |
| 9 | ACTR3B, Adaptor protein 2, AGAP1, ALDH1L1, ANG, AP2A1, ARL6, | 54 | Cell |
| | BDNF, Ca2+, CALB2, Calbindin, CAR ligand-CAR-Retinoic acid-RXRα, | | Signaling, |
| | CCNE2, CD209, CDKN2A, CHCHD10, CLTCL1, DACH1, DAP, ERBB2, | | Molecular |
| | FAM131B, FFAR2, FGF1, FGF5, FKBP1A, Focal adhesion kinase, GJC1, | | Transport, |
| | GSTM4, GYPC, HBD, HBQ1 (includes EG:3049), HIP1, HNF4A, HOXA2, | | Vitamin and |
| | IL6, INVS, IRF6, JPH2, K+, LBP, LRRN2, MIR103-1 (includes EG:406895), | | Mineral |
| | MIR181B2, MIR24-1 (includes EG:407012), MYCN, Na+, NDN, NECAP1, | | Metabolism |
| | NPHP1, NRG, P2RY4, PCDH19, PHKB, RASIP1, RPS10, Ryr, S100A11, | | |
| | SCGB1A1, SEC61B, SLC24A3, SLC35A2, TBRG1, TFAP2B, TFAP2C, | | |
| | TM6SF1, TMEM33, TNKS1BP1, TOB1, VCL, ZNF609 | | |
| 10 | Actin, ADD1, Akt, ANG, Arf, ASAH2, CCR3, CCR7, CD3E, CEACAM1, | 52 | Cardiovascular |
| | CES1 (includes EG:1066), CXCR2, DAB1, DBC1, DMD, E2f, EFNB2, | | System |
| | EIF4B, EPHB3, EPHB4, EPHB6, EPS8L1, ERK1/2, F Actin, FER (includes | | Development |
| | EG:2241), GPC3, hCG, HIPK2, HIST1H2AE (includes EG:3012), | | and Function, |
| | HIST1H2BJ (includes EG:8970), Histone h3, IFIT1, II8r, Insulin, Interferon | | Organismal |
| | alpha, Jnk, KLRG1, LBP, LDL, LIPE, MADCAM1, MAG, NCF1, NCF4, | | Development, |
| | NWASP, PDGF BB, PDGF-AA, Pkc(s), PLIN1, POU3F1, PROK2, | | Developmental |
| | PROKR1, PRX, Ras homolog, SBF1, SCARF1, SLC4A4, SNTG1, | | Disorder |
| | SNX33, SPTA1, SPTB, SUV39H1, TCL1A, TGM1, TIE1, TMEFF2, TNS1, | | |
| | Tropomyosin, UTRN, WAS | | |
| 11 | ALAD, butyric acid, C10ORF10, C15ORF63, CD209, CELF1, CPA3, | 51 | Cellular |
| | CTNNB1, CTRL, D4S234E, DOCK2, ELMO1, EN1, FABP7, GPD1, HBD, | | Development, |
| | HOMER3, HOXD3, HTT, ICA1, IER2, IFIT1, IFNG, II8r, ITGAX, KCNC3, | | Endocrine |
| | KCTD17, LIX1L, MIR124-1 (includes EG:406907), MIR206 (includes | | System |
| | EG:406989), MKKS, MSR1, MYL4, MYLK2, Myosin Light Chain Kinase, | | Development |
| | NEUROD1, NEUROG1, NFIX, NKX2-2, norepinephrine, NOTCH4, NPTX1, | | and Function, |
| | NPTXR, PAX6, peptidase, PI4KA, QKI, RCN2, RFX1, SBF1, SCG5, | | Endocrine |
| | SEC61B, SIK1, SLC11A1, SLC26A2, SP1, STK16, STX12, TCF20, | | System |
| | TCF7L1, TEX261, TGFB1, TGM1, TINAGL1, TNF, TREM2, TSPAN7, | | Disorders |
| | TYR, USH1C, WWTR1 | | |

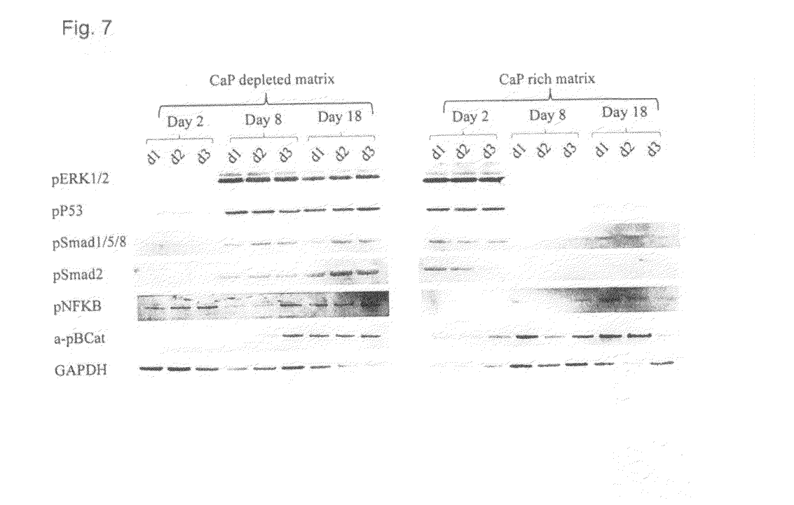

To investigate whether these pathways were differentially activated in CPDM versus CPRM, we probed for phosphorylated proteins that are key messengers of these pathways with Western blot. Indeed, differential expression of the phosphorylated protein between CPDM and CPRM was found for all proteins tested (FIG. 3C and FIG. 7). Within each condition p-Erk1/2, p-p53, p-Smad1/5/8 and p-Smad2 displayed very similar temporal profiles, with a high expression two days after implantation, followed by a decline after one week and an increase after 18 days. Intriguingly, pCREB protein expression followed the same dynamics in CPRM. Activated beta catenin showed an analogous profile of p-Erk, p-p53 and p-Smads in CPDM. The differential expression of the phosphorylated proteins between CPRM and CPDM validate the activation of the signaling pathways as identified by our gene network analysis.

Development of an osteoinductive growth factor cocktail. To further confirm our hub gene network, we hypothesized that in vitro activation of the identified signaling pathways may significantly promote osteogenic differentiation of hPDCs. Currently, in vitro osteogenic differentiation in human MSCs is induced by serum containing growth medium supplemented with dexamethasone, beta glycerophosphate and ascorbic acid (1, 2). This osteogenic medium (OM) has been optimized for bone marrow derived stem cells (3) but is inconsistent to induce in vitro osteogenesis in hPDCs (4, 5).

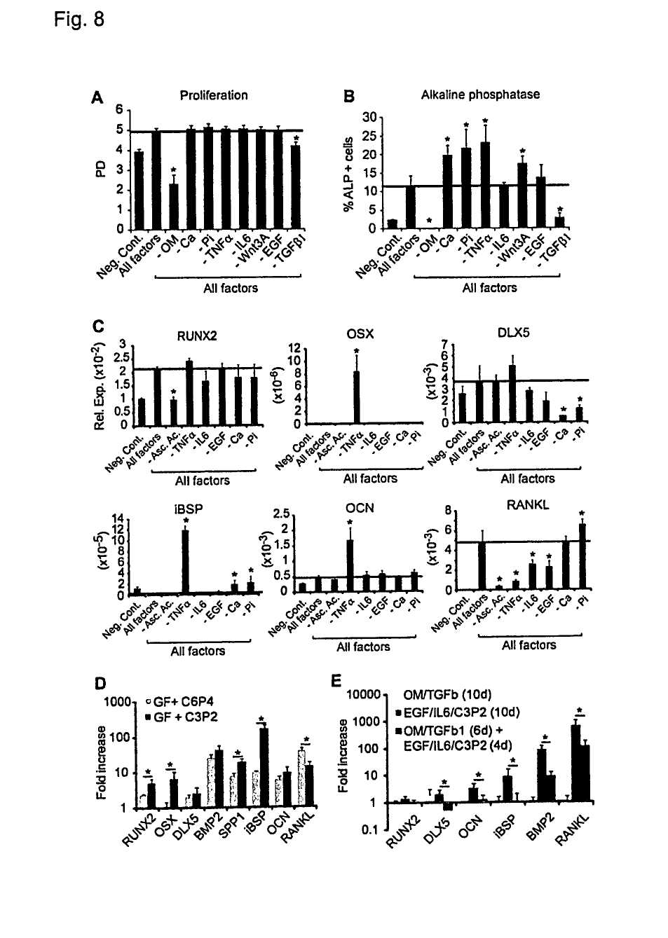

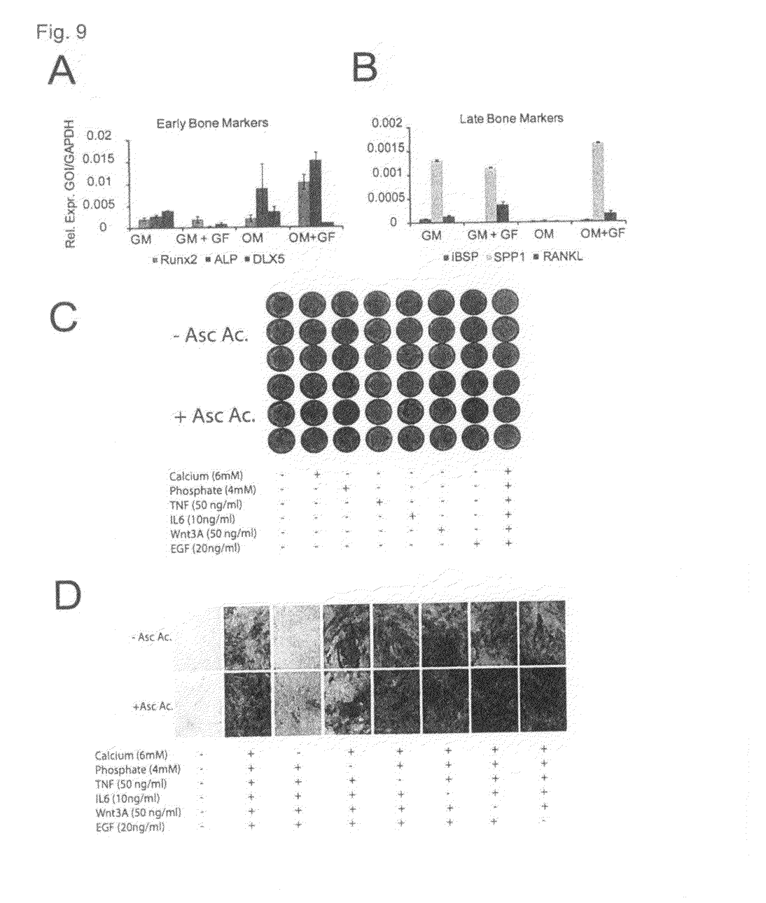

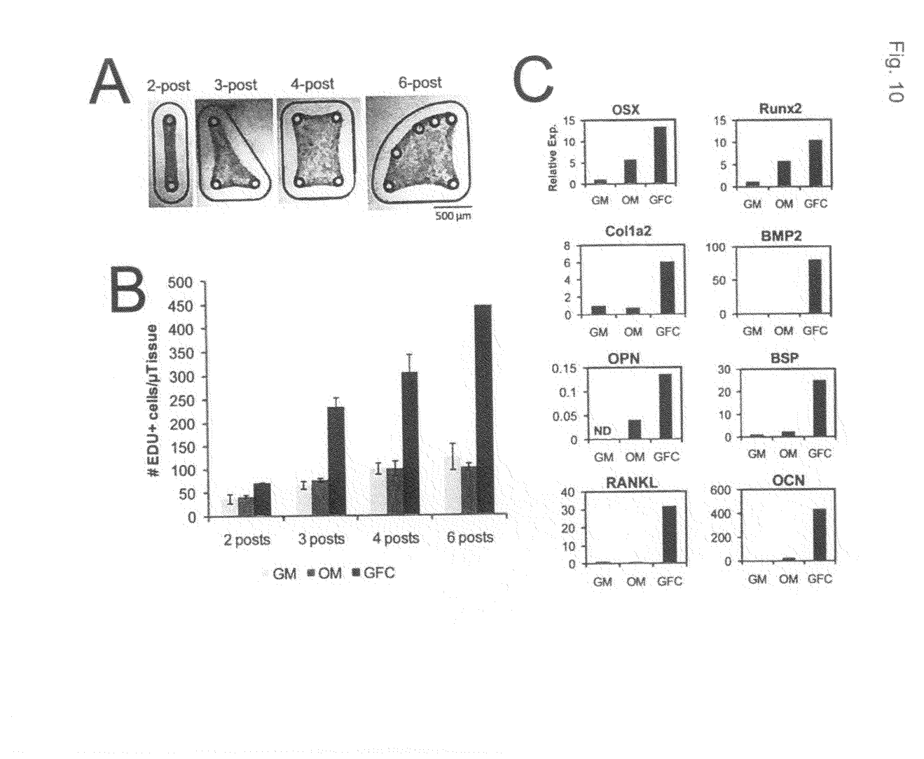

Inspired by Takahashi and Yamanaka's work on identifying factors for reprogramming dermal fibroblasts in stem cells (11), we adopted a similar “leave-one-out” strategy to identify key components that stimulate proliferation and differentiation of hPDCs in vitro. Based on the hub gene network, we selected TNFα, IL6, EGF, TGFβ1 and Wnt3A ligands together with calcium and phosphate ions as factors to induce osteogenic differentiation. Because gene topology suggested that hub genes may accelerate rather than induce osteogenic differentiation, we considered OM as induction medium. OM supplemented with all factors served as a reference to evaluate the impact of a single factor on proliferation, ALP expression or gene expression after exclusion from the cocktail. Negative regulation of a metric in absence of one factor indicates that this factor is important for this metric. Following this logic, we identified two factors, OM and TGFβ1, being strong inducers of proliferation and ALP activity of hPDCs (FIG. 8A and B). Interestingly, OM supplemented with all factors promoted gene expression of RUNX2 and ALP after one week of stimulation (FIG. 9A) suggesting early differentiation. However, OM alone did not enhance RUNX2 transcription and even reduced basal expression levels of later bone markers, iBSP, SPP1 and RANKL (FIG. 9B). OSX expression was undetectable in all conditions (data not shown). These data indicate that OM interfered with the progression of an osteoprogenitor to a mature osteoblast.

To overcome the inhibitory effect of OM on later stages of osteoblastogenesis, we considered to explore a two stage protocol wherein hPDCs were treated with OM and TGFβ1 to stimulate proliferation and ALP activity. After six days, medium was changed to growth medium supplemented with six factors (ascorbic acid, TNFα, IL6, EGF, Ca, Pi) minus one factor for 4 days. At this stage, ascorbic acid was included as a factor, because it promoted ALP activity (FIG. 9C) and mineralization (FIG. 9D) in vitro. To evaluate osteoblast differentiation, expression of several bone markers which were previously upregulated in vivo (FIG. 1B) was measured. Gene expression levels of RUNX2 were decreased when ascorbic acid was omitted from the mix. Removing TNFα from the cocktail enhanced expression levels of OSX, iBSP, and OC suggesting that TNFα is a strong inhibitor of osteogenic differentiation (FIG. 8C). However, cells treated with medium devoid of TNFα, ascorbic acid, IL6 or EGF ligands displayed lower levels of RANKL expression (FIG. 8C). Furthermore, EGF, calcium and phosphate were required for DLX5 transcription, but at the used concentrations, calcium and phosphate decreased iBSP mRNA levels (FIG. 8C). These data suggested to omit TNFα from the mix, and to reduce the concentration of calcium and phosphate ions. Indeed, expression levels of RUNX2, OSX, SPP1, iBSP were significantly higher in hPDCs treated with a two staged stimulation protocol containing 3 mM Ca and 2 mM Pi, instead of 6 and 4 mM respectively (FIG. 8D).

To test whether a two stage protocol yields better osteogenic differentiation in vitro as compared to a single stage protocol, hPDCs from four different donors were either stimulated with stimulation medium of the first stage (OM and TGFβ1), second stage (GM supplemented with EGF, IL6, Ca/Pi) for 10 days or two stage (0M/TGFβ1 for 6 days followed by GM/ascorbic acid/EGF/IL6/Ca/Pi for 4 days). Surprisingly, gene expression levels for multiple bone markers (DLX5, BMP2, iBSP, OCN and RANKL) were higher when treated with the second stage growth factor (GF) mix only as compared to the two stage protocol (FIG. 8E). These data prompted us to abandon a two stage protocol and to assess proliferation and osteogenic differentiation of hPDCs after treatment with a GF/ion cocktail medium as defined in table 6.

TABLE 6
Composition of Growth Factor (GF) medium = Growth
medium (GM) + Growth Factor Cocktail)

| | Concentration | | Company |
|---|---|---|---|
| Growth medium | | | |
| Dulbecco's Modified | 4.5 g/dl Glucose | | Invitrogen |
| Eagle Medium | | | |
| Fetal Bovine Serum | 10% | | Gibco |
| Penn/Strep | 1% | | Invitrogen |
| Growth Factor Cocktail | | | |
| EGF | 20 | ng/ml | RD systems |
| IL6 | 10 | ng/ml | RD systems |
| TGFb1 | 10 | ng/ml | StemRD |
| Ascorbic Acid | 50 | μM | Sigma |

| | | |
|---|---|---|
| Calcium ions | 3 mM in HBS buffer | Sigma |
| Phosphate ions | 2 mM in HBS | Sigma |

In vitro activation of early osteogenic gene networks promotes osteogenic differentiation in hPDCs in vitro and in vivo.

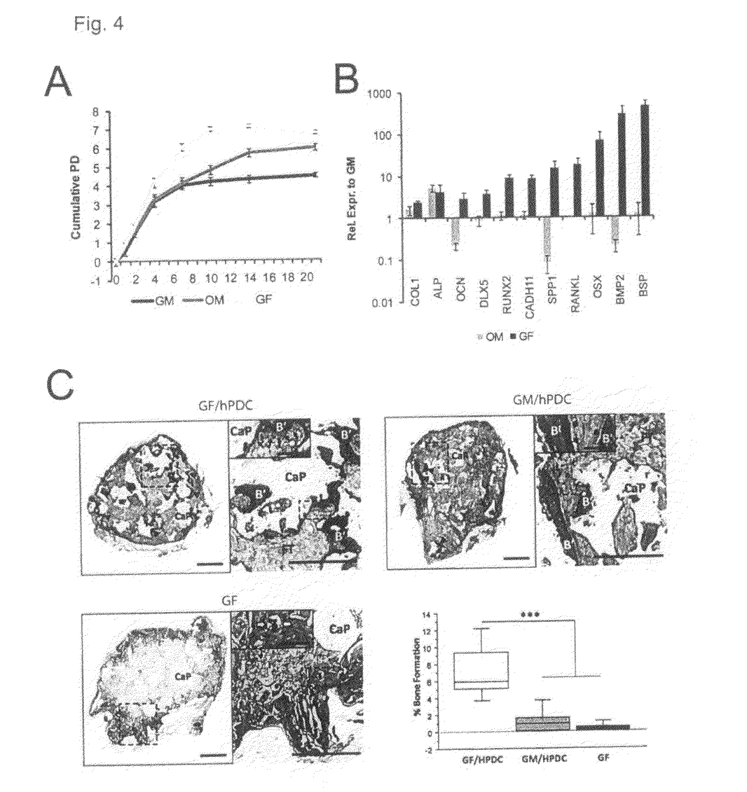

The defined growth factor/ion cocktail (Table 6) enhances proliferation (FIG. 4A) and osteogenic differentiation (FIG. 4B) of hPDCs in vitro. hPDCs treated with GF medium proliferated for 7 (SEM: ±0.1) population doublings, whereas hPDCs in OM reached 4.8 (SEM: ±0.2) population doublings after 11 days. Interestingly, ALP activity and gene expression of COL1 and ALP (FIG. 4B) was comparable in OM treated and GF medium treated cells. In contrast, mRNA levels of other bone markers characteristic for early (DLX5, OSX, RUNX2, and BMP2), intermediate (SPP1, BSP) and late (RANKL and OCN) stages of osteoblast differentiation were significantly higher expressed in GF treated cells as compared to OM treated cells (FIG. 4B). These data demonstrate that in vitro activation of selected hub genes primes hPDCs to the osteogenic commitment more efficiently than OM.

We next investigated whether pretreatment of hPDCs with GF medium would rescue or enhance ectopic bone formation in vivo. Briefly, hPDCs were seeded on CPDM or CPRM carriers, pretreated with GM or GF medium for 11 days and subcutaneously implanted in nude mice for 8 weeks. GF medium could not rescue bone formation in CPDM carriers, but increased the amount of bone tissue deposited by hPDCs engrafted in CPRM by approximately 6-fold as compared to hPDCs seeded on CPRM and cultured in GM (FIG. 4C). CPRM carriers incubated in GF medium prior to implantation did not show any signs of bone formation suggesting that the fraction of growth factors or ions adherent to the scaffold did not induce bone formation in host cells after implantation (FIG. 4C).

### Example 3: Potency of GFC on Proliferation and Osteogenic Differentiation in 3D

To evaluate the potency of the GFC on proliferation and osteogenic differentiation in 3D, we seeded hPDCs in a 3D collagen type I/fibrinogen gel in a newly developed microtug device. This device is an array of differently shaped micro wells made of polydimethylsulfoxide (PDMS) that contain 160 μm tall cantilever posts (2, 3, 4, or 6 posts) spaced out in different geometries. After seeding the cell/matrix mixture in the device, hPDCs spread out, exert contractile forces on the gel, and remodel the collagen matrix. As such, the collagen/fibrinogen matrix and cells compact into microtissues that are constrained by the posts (FIG. 10 A). This way, the impact of mutual interactions of cell generated forces and the surrounding extracellular matrix on cell function can be investigated in 3D.

Using this device, we tested if OM and GM stimulate proliferation of hPDCs in 3D. Microtissues were formed and cultured in GM, OM or GFC for 4 days. After 4 days, the cells were pulsed with 5-ethynyl-2′-deoxyuridine (EDU), a thymidine substitute that incorporates in the nucleus of proliferating cells, for 24 h. Subsequently cells were fixed and processed to visualize EDU incorporation. Quantification of the number of EDU positive cells shows that microtissues treated with GFC contain more EDU positive cells as compared to microtissues cultured in GM or OM (FIG. 10 B) indicating that, independent of the geometry of the microtissues, the GFC strongly promotes proliferation in 3D.

To assess osteogenic differentiation, microtissues were treated with GM, OM or GFC for 3 weeks, followed by RNA extraction and quantitative PCR to measure gene expression levels of bone markers. Consistent with the data obtained in 2D cultures, the GFC enhances gene expression levels of early (OSX, RUNX2), intermediate (Col1a2, OPN and BSP), and late (RANKL, OCN) stage osteoblast markers more efficiently than OM (FIG. 10 C). In addition, the GFC also strongly promotes BMP2 gene expression, a signaling molecule that drives the process of osteoinduction in vitro and in vivo (FIG. 10 C). Taken together, these data indicate that the GFC is a potent stimulator of proliferation and osteogenic differentiation of hPDCs in 3D collagen gels.

### REFERENCE LIST

1. Jaiswal, N., Haynesworth, S. E., Caplan, A. I. & Bruder, S. P. (1997) J. Cell Biochem. 64, 295-312.

2. Pittenger, M. F., Mackay, A. M., Beck, S. C., Jaiswal, R. K., Douglas, R., Mosca, J. D., Moorman, M. A., Simonetti, D. W., Craig, S. & Marshak, D. R. (1999) Science 284, 143-147.

3. Jaiswal, N., Haynesworth, S. E., Caplan, A. I. & Bruder, S. P. (1997) J. Cell Biochem. 64, 295-312.

4. Eyckmans, J. & Luyten, F. P. (2006) Tissue Eng.

5. Roberts, S. J., Chen, Y., Moesen, M., Schrooten, J. & Luyten, F. P. (2011) Stem Cell Res. 7, 137-144.

6. Eyckmans, J., Roberts, S. J., Schrooten, J. & Luyten, F. P. (2010) J. Cell Mol. Med. 14, 1845-1856.

7. Chaff, Y. C., Roberts, S. J., Schrooten, J. & Luyten, F. P. (2011) Tissue Eng Part A 17, 1083-1097.

8. Eichler, G. S., Huang, S. & Ingber, D. E. (2003) Bioinformatics. 19, 2321-2322.

9. Huang, d. W., Sherman, B. T. & Lempicki, R. A. (2009) Nat. Protoc. 4, 44-57.

10. Eichler, G. S., Huang, S. & Ingber, D. E. (2003) Bioinformatics. 19, 2321-2322.

11. Takahashi, K. & Yamanaka, S. (2006) Cell 126, 663-676.

1. A method for inducing cells to proliferate and differentiate into cells with a osteogenic phenotype, the method comprising culturing cells in a medium comprising about 2 ng/ml to about 200 ng/ml EGF, about 1 ng/ml to about 100 ng/ml IL6, and about 1 ng/ml to about 100 ng/ml TGFβ1.

2. The method of claim 1 , wherein the medium comprises about 20 ng/ml EGF, about 10 ng/ml IL6, and about 10 ng/ml TGFβ1.

3. The method of claim 1, wherein the medium contains a calcium ion concentration ranging from about 0.3 mM to about 12 mM.

4. (canceled)

5. The method of claim 1, wherein the medium contains a serum concentration ranging from 0% to about 20%.

6. (canceled)

7. The method of claim 1, wherein the medium contains from about 10⁻⁴ M to about 10⁻⁷ M ascorbic acid.

8. (canceled)

9. The method of claim 1, wherein the medium contains a phosphate ion concentration ranging from about 0.2 mM to about 8 mM.

10. (canceled)

11. The method of claim 1, wherein the cells are cultured for at least four days.

12. (canceled)

13. The method of claim 1, wherein the cells are cultured in a medium which additionally comprises TNFα in a first period, wherein said first period is maximum 4 days.

14. (canceled)

15. The method of claim 1, wherein the cells that are cultured with the medium comprising EGF, IL6 and TGFβ1 are stem cells.

16.-20. (canceled)

21. Cells produced according to the method recited in claim 1.

22. A composition, comprising cells in a culture medium comprising about 2 ng/ml to about 200 ng/ml EGF, about 1 ng/ml to about 100 ng/ml IL6 and about 1 ng/ml to about 100 ng/ml TGFβ1, wherein the cells express a primitive mesenchymal phenotype in the culture medium.

23. The composition of claim 22, wherein the medium is comprised of about 20 ng/ml EGF, about 10 ng/ml IL6 and about 10 ng/ml TGFβ1.

24. The composition of claim 22, wherein the medium further comprises serum in a concentration from 0% to about 20%.

25. (canceled)

26. The composition of claim 22, wherein the medium further comprises about 10⁻⁴ M to about 10⁻⁷ M ascorbic acid.

27.-29. (canceled)

30. A pharmaceutical composition comprising the cells produced according to the method recited in claim 1.

31. A method of treatment comprising administering a therapeutically effective amount of the cells produced according the method recited in claim 1 to a subject with a bone disorder.

32.-33. (canceled)

34. The method of claim 31, wherein said bone disorder is a bone fracture or a non healing bone defect.

35. The method of claim 31, wherein the subject is a human patient.

36. The method of claim 31, further comprising administering non-cellular material to said subject.

37. The method of claim 36, wherein the cells and the non-cellular material are combined in vitro to form an implantable graft.


SEQUENCE LISTING (16 sequences)

SEQ ID NO: 1 - DNA, 20 bp, Artificial Sequence
  Primer Sequences
agtgacctttcagcctccaa

SEQ ID NO: 2 - DNA, 20 bp, Artificial Sequence
  Primer Sequences
gggaaaagggagggtaatca

SEQ ID NO: 3 - DNA, 21 bp, Artificial Sequence
  Primer Sequences
ccgaagaaaatggagatgaca

SEQ ID NO: 4 - DNA, 20 bp, Artificial Sequence
  Primer Sequences
cctctccatagcccagtgtt

SEQ ID NO: 5 - DNA, 19 bp, Artificial Sequence
  Primer Sequences
gtgcagcctttgtgtccaa

SEQ ID NO: 6 - DNA, 19 bp, Artificial Sequence
  Primer Sequences
gctcacacacctccctcct

SEQ ID NO: 7 - DNA, 20 bp, Artificial Sequence
  Primer Sequences
ccggagcacgattgtctatg

SEQ ID NO: 8 - DNA, 20 bp, Artificial Sequence
  Primer Sequences
ctcgacgttttcaccgttgt

SEQ ID NO: 9 - DNA, 20 bp, Artificial Sequence
  Primer Sequences
tcaacattgacgcactccag

SEQ ID NO: 10 - DNA, 20 bp, Artificial Sequence
  Primer Sequences
gaggcatccacgacctcata

SEQ ID NO: 11 - DNA, 20 bp, Artificial Sequence
  Primer Sequences
taaattctgggagggcttgg

SEQ ID NO: 12 - DNA, 20 bp, Artificial Sequence
  Primer Sequences
gatgcctaggaggcaaaagc

SEQ ID NO: 13 - DNA, 21 bp, Artificial Sequence
  Primer Sequences
gatcctcttcaggaggtgagg

SEQ ID NO: 14 - DNA, 20 bp, Artificial Sequence
  Primer Sequences
acagctcccgggtgtttatc

SEQ ID NO: 15 - DNA, 20 bp, Artificial Sequence
  Primer Sequences
cctttcaaggagctgtgcaa

SEQ ID NO: 16 - DNA, 19 bp, Artificial Sequence
  Primer Sequences
tgggaaccagatgggatgt